(19) United States
(12) Patent Application Publication
(10) Pub. No.: US 2025/0333696 A1
(43) Pub. Date: Oct. 30, 2025

(54) DNA-CHIMERIC ANTIGEN RECEPTOR T CELLS FOR IMMUNOTHERAPY

(71) Applicant: ARIZONA BOARD OF REGENTS ON BEHALF OF ARIZONA STATE UNIVERSITY, Scottsdale, AZ (US)

(72) Inventors: Hao YAN, Chandler, AZ (US); Bo NING, Tempe, AZ (US)

(21) Appl. No.: 19/188,325

(22) Filed: Apr. 24, 2025

Related U.S. Application Data
(62) Division of application No. 16/966,264, filed on Jul. 30, 2020, now Pat. No. 12,338,459.
(60) Provisional application No. 62/625,964, filed on Feb. 2, 2018.

(51) Int. Cl. C12N 5/0783; A61K 40/11; A61K 40/31; A61K 40/42; B82Y 5/00; C07K 14/705; C12N 15/115

(52) U.S. Cl. CPC C12N 5/0636; A61K 40/11; A61K 40/31; A61K 40/4204; A61K 40/4205; A61K 40/421; A61K 40/4211; A61K 40/4255; A61K 40/4257; A61K 40/4258; C07K 14/70517; C12N 15/115; A61K 2239/31; A61K 2239/38; A61K 2239/48; A61K 2239/49; A61K 2239/55; B82Y 5/00; C07K 2317/70; C07K 2319/03; C07K 2319/50; C12N 2310/16; C12N 2310/3513; C12N 2510/00

(57) ABSTRACT

In certain embodiments, this disclosure provides methods to generate DNA, RNA and/or DNA-peptide nanostructures based chimeric antigen receptor (CAR) T cell (engineered T cell) for cancer immunotherapy, and compositions made by these methods.

### RELATED APPLICATIONS

This application is a Divisional of U.S. application Ser. No. 16/966,264, filed Jul. 30, 2020, which was a 371 National Stage Entry of PCT/US2019/016590, filed Feb. 4, 2019, which claims the benefit of U.S. Provisional Application No. 62/625,964, filed on Feb. 2, 2018, the entire disclosure of which is incorporated herein by reference.

### SEQUENCE LISTING

The instant application contains a Sequence Listing which has been submitted electronically in XML format and is hereby incorporated by reference in its entirety. Said XML copy, created on Apr. 23, 2025, is named G8118-01203_SL.xml and is 412,170 bytes in size.

### BACKGROUND

Cancer immunotherapy is an emerging field that has demonstrated significant promise in recent years. Perhaps the most exciting of these approaches has been the use of peripheral blood T cells genetically modified to express chimeric antigen receptor (CAR) genes for use in hematological “liquid cancers.” However, in the much more common case of solid tumor cancers, CAR T cells meet obstacles including physical barriers, loss of antigen and immunosuppressive environments. Accordingly, cancer therapies that overcome these obstacles are needed.

Adoptive cell transfer denotes the transfer of immunocompetent cells for the treatment of cancer or infectious diseases (June, C. H., ed., 2001, In: Cancer Chemotherapy and Biotherapy: Principles and Practice, Lippincott Williams & Wilkins, Baltimore; Vonderheide et al., 2003, Immun. Research 27:1-15). Adoptive cell therapy is a strategy aimed at replacing, repairing, or enhancing the biological function of a damaged tissue or system by means of autologous or allogeneic cells, and thus can be used in cancer immunotherapy.

### SUMMARY

In certain aspects, this disclosure provides a method to generate an engineered T cell for cancer immunotherapy. In some aspects, the engineered T cell comprises a DNA, RNA and/or DNA-peptide nanostructure-based chimeric antigen receptor (CAR) domain.

In certain aspects, this disclosure provides an engineered T cell comprising: an expressed engineered chimeric antigen receptor which comprises an extracellular adaptor protein; a protein tag bound to said adaptor protein; a first oligonucleotide connected to said protein tag; a second oligonucleotide wherein a portion of the second oligonucleotide sequence is complementary to a portion of the first oligonucleotide sequence; and a targeting agent connected to the second oligonucleotide, where the targeting agent comprises one or a plurality of targeting molecules. In certain aspects, the targeting agent comprises a DNA origami nanostructure comprising a central polynucleotide strand and a first staple strand which comprises the second oligonucleotide sequence and a plurality of second staple strands which comprises one or a plurality of third distinct oligonucleotide sequences; and one or more targeting molecules connected to one or a plurality of fourth distinct oligonucleotide sequence(s). In certain aspects, a portion of the third distinct oligonucleotide sequence is complementary to a portion of the fourth oligonucleotide sequence.

In certain aspects, the expressed engineered chimeric antigen receptor comprises: a signaling polypeptide domain; a transmembrane polypeptide domain; a spacer polypeptide domain; a costimulatory polypeptide domain; and an adaptor protein tag domain. In certain aspects, the expressed engineered chimeric antigen receptor further comprises a fluorescent protein domain. The fluorescent protein domain is selected from green fluorescent protein (GFP), blue fluorescent proteins (EBFP, EBFP2, Azurite, mKalama1), cyan fluorescent proteins (ECFP, Cerulean, CyPet, mTurquoise2), yellow fluorescent proteins (YFP, Citrine, Venus, YPet), and bilirubin-inducible fluorescent proteins (UnaG, dsRed, eqFP611, Dronpa, TagRFPs, KFP, EosFP, Dendra, and IrisFP). In certain embodiments, the fluorescent protein domain is a fluorescent protein having a major excitation peak of about 488 nm, and an emission of about 509 nm.

In certain aspects, the polynucleotide sequence encoding the expressed engineered chimeric antigen receptor comprises an antibiotic resistant gene. The purpose of the antibiotic resistant gene is to identify the transfected cells in cell culture. In some embodiments, the antibiotic resistant gene is a puromycin gene. In some embodiments, the antibiotic resistant gene is selected from puro, pac, pUNO1-pac, or pEGFP-puro.

In certain aspects, the signaling polypeptide domain is CD3Z.

In certain aspects, the transmembrane polypeptide domain is CD8.

In certain aspects, the costimulatory domain is selected from: CD28, 4-1BB, OX-40, and combinations thereof.

In certain aspects, the spacer polypeptide domain is a repeat of the sequence (Gly-Gly-Gly-Gly-Ser)ₙ, where n is an integer selected from 1 to 8 (SEQ ID NO: 1).

In certain aspects, the adaptor protein tag domain is selected from the SNAP-Tag™, CLIP-Tag™, or HALO-Tag™ adaptor proteins.

In certain aspects, the signaling polypeptide domain is CD3ζ, the transmembrane polypeptide domain is CD8, the costimulatory domain is 4-1BB, the spacer polypeptide domain is (Gly-Gly-Gly-Gly-Ser)₄ (SEQ ID NO: 2), and the adaptor protein tag domain is SNAP-Tag™ adaptor protein.

In certain aspects, the targeting molecule is selected from: an aptamer, a synbody, and an antibody or fragment thereof. In certain aspects, the antibody fragment is a scFv. In certain aspects. The aptamer is scg8 having SEQ ID NO: 10.

In certain aspects, the targeting molecule comprises a matrix metalloproteinase.

In certain aspects, the targeting molecule comprises a cytokine, chemokine, or a combination thereof.

In certain aspects, the targeting molecule comprises an inhibitory pathway overcoming agent. In certain aspects, the inhibitory pathway overcoming agent is selected from an anti-PD-1L antibody, an anti-PD-1L aptamer, an anti-CTLA4 antibody, or an anti-CTLA4 aptamer.

In certain aspects, the T cell is selected from a natural killer T cell, a regulatory T cell, a helper T cell, a cytotoxic T cell, a memory T cell, a gamma delta T cell and a mucosal invariant T cell.

In certain aspects, this disclosure relates to a vector encoding a chimeric antigen receptor described herein.

In certain aspects, this disclosure relates to a method of preparing an engineered T cell comprising the steps of: inserting a DNA sequence which encodes for the CAR polypeptide into a virus; contacting the virus with a T cell to form or produce a viral-infused T cell; growing the viral-infused T cells to produce an adaptor T cell expressing the CAR comprising the extracellular adaptor protein; isolating the adaptor T cell; contacting the extracellular adaptor protein of the isolated adaptor T cells with a first oligonucleotide functionalized with a cognate protein tag; forming a complex between the extracellular adaptor protein of the adaptor T cells with the cognate protein tag to form a first oligonucleotide-functionalized adaptor T cell; contacting the first oligonucleotide-functionalized adaptor T cell with a second oligonucleotide comprising a targeting agent under appropriate conditions to form a hybridization complex between a portion of the first linker oligonucleotide and a portion of the second linker oligonucleotide.

In certain aspects, this disclosure relates to a method of preparing the engineered T cell comprising the steps of: inserting the DNA sequence which encodes for the CAR polypeptide into a virus; contacting the virus with a T cell to form or produce a viral-infused T cell; growing the viral-infused T cells to produce an adaptor T cell expressing the CAR comprising the extracellular adaptor protein; isolating the adaptor T cell; contacting the extracellular adaptor protein of the isolated adaptor T cells with a first oligonucleotide functionalized with a cognate protein tag of the adaptor T cells with the cognate protein tag to form a first oligonucleotide-functionalized adaptor T cell; contacting the first oligonucleotide-functionalized adaptor T cell with a first staple strand of a DNA origami nanostructure comprising a central polynucleotide sequence, a first staple strand, and one or a plurality of third distinct oligonucleotide sequences to form a hybridization complex between a portion of the first oligonucleotide and a portion of the one or a plurality of third distinct oligonucleotide sequences; contacting said DNA origami nanostructure with one or a plurality of targeting molecules comprising a second oligonucleotide sequence under appropriate conditions to form a hybridization complex between a portion of the sequences of the one or a plurality of third distinct oligonucleotide sequences and a portion of the second oligonucleotide sequence. In certain aspects, the virus is selected from a lentivirus, retrovirus or adeno-associated virus.

In certain aspects, this disclosure relates to a method of activating an engineered T cell comprising contacting a cancer cell with an engineered T cell described herein. In certain aspects, the cancer cell is selected from a hematological cancer or a tumor cell. In some aspects, the cancer cell is a T lymphoid blastoma. In some aspects, the T lymphoid plastoma is a CCRF-CEM cell. In some aspects, the cancer cell is a breast cancer cell or a lung cancer cell.

In certain aspects, this disclosure relates to a method of killing a cancer cell comprising contacting a cancer cell with an engineered T cell described herein. In certain aspects, the cancer cell is selected from a hematological cancer or a tumor cell. In some aspects, the cancer cell is a T lymphoid blastoma. In some aspects, the T lymphoid plastoma is a CCRF-CEM cell. In some aspects, the cancer cell is a breast cancer cell or a lung cancer cell.

In certain aspects, this disclosure relates to a method of killing a cancer cell comprising the steps of contacting a cancer cell with a DNA nanostructure comprising a plurality of staple strands where a first staple strand is unhybridized and a second staple strand is hybridized to a fourth oligonucleotide which is chemically conjugated to a targeting molecule, and said targeting molecule binds to said cancer cell; then contacting the DNA nanostructure with an engineered T cell comprising a first oligonucleotide which is complementary to the first staple strand on the DNA nanostructure to form a hybridization complex between the first oligonucleotide and the first staple strand to bring the engineered T cell in local proximity to the cancer cell and begin a cytolytic mechanism.

In certain aspects, this disclosure relates to a composition comprising the engineered T cell as described herein with a pharmaceutically acceptable excipient. In some aspects, this disclosure relates to a method for activating engineered T cells and/or killing cancer cells in a subject having or suspected of having cancer by administering to said subject said composition.

In certain aspects, this disclosure provides a single- or multiple-target antigen-specific CAR T cells.

In certain aspects, this disclosure provides a single- or multiple-target antigen-specific ECM degrading CAR T cell.

In certain aspects, this disclosure provides a single- or multiple-target antigen-specific cytokine expressing CAR T cell.

In certain aspects, this disclosure provides a single- or multiple-target antigen-specific inhibitory pathway overcoming CAR T cell.

### BRIEF DESCRIPTION OF DRAWINGS

FIG. 1. The immunosuppressive tumor microenvironment presents multiple challenges for chimeric antigen receptor (CAR) T cells. In addition to proper trafficking and successful infiltration, there are additional hurdles presented upon their arrival in a solid tumor microenvironment. Including low pH, hypoxia, suppressive immune cells or cytokines and co-inhibitory molecules including PD-1 or CTLA4. Failure to overcome the presence of these negative elements results in inhibition of the T cell activity and unabated tumor growth.

FIG. 2A. Structural DNA nanotechnology principles are used to create DNA nanostructure motifs (top) which are used to create periodic 2D arrays and corresponding AFM images (bottom): double-crossover, 3×4, 4×4, and 6×4 DNA tiles.

FIG. 2B: Structural DNA nanotechnology principles are used to create DNA origami nanostructures (top, schematic drawings of the structures; bottom, corresponding AFM or TEM images): 2D DNA origami smiley face, 3D DNA origami in the shape of a gear, curved single-layer 3D origami in the shape of a vase, and DNA origami gridiron.

FIGS. 3A-3B. Advantages of engineered T cells for cancer immunotherapy. FIG. 3A. Current CAR T structures and issues have resulted in failure of CAR T therapy in solid tumors. Hostile microenvironments decreased therapy effects of CAR T cells. FIG. 3B: Novel T cell toolkit using DNA nanotechnology (“engineered T cell”). DNA origami carrying targeting and functional molecules helps engineered T cells overcome obstacles in solid tumor. This strategy bypasses the difficulties of genetic engineering and allow for rapid, modular generation of targeting engineered T cells using a wider range of targeting ligands than previously possible.

FIG. 4. Approach overview of engineered T cell in solid tumor therapy. Using engineered T cells can solve specific problems by assembly different functional molecules. DNA nanotechnology promotes T cell infiltration overcomes the hostile environment in solid tumor.

FIG. 5A-FIG. 5B. Design models (top row) and corresponding AFM images of two representative DNA nanostructures. (FIG. 5A) DNA nanostructures with close-packed helixes and (FIG. 5B) DNA nanostructures with wireframe patterns.

FIG. 6. Strategies of increasing engineered T cell infiltration in solid tumors. T cells expressing adaptor-CAR proteins link different targeting molecules including DNA aptamers, dipeptide synbodies, or single chain antibody to generate engineered T cells. Single or multiple targeting molecules recognize tumor cells and activate engineered T cells. DNA nanostructures are used to capture engineered T cell in tumor microenvironment. Tumor-trigged DNA nanocages open and release chemokines or VEGF-C to recruit engineered T cells to solid tumor area.

FIG. 7A-FIG. 7D. Design of lentiviral vector encoding adaptor-CAR proteins. (FIG. 7A) Second generation engineered T cell comprising an engineered CAR comprising: a SNAP-Tag™ protein following with CD8 transmembrane domain (TM), CD3ζ activation domain and different signaling domains: CD28, 4-1BB or OX-40; (FIG. 7B) Second generation engineered T cell with a CD8 leader sequence (L). (FIG. 7C) Third generation engineered T cell comprising an engineered CAR comprising: a SNAP-Tag™ tag following with TM, CD3 activation domain and two signaling domains: CD28 with 4-1BB or OX-40; (FIG. 7D) Third generation T cell comprising an engineered CAR with CD8 leader sequence.

FIG. 8. Illustration of a synbody affinity ligand.

FIG. 9. Workflow to design appropriate synbodies for a target protein. The target protein is screened against a peptide array to identify low-affinity peptide hits. The hits are conjugated to a bifunctional synbody scaffold in pairwise combination to produce a library of synbodies.

FIG. 10. The schematics show the design of bivalent aptamer of thrombin. Two different aptamers are organized together by a DNA scaffold linker with optimized distances.

FIGS. 11A-11C. Protein screening against in situ synthesized peptide microarrays. FIG. 11A: Layout of peptide libraries on slide. The 125K peptide library is synthesized as individually addressable arrays, A1 through A24. FIG. 11B TNFA (x-axis) and TNFA+TNFR1 complex (y-axis) binding to 125k peptide microarray. Each spot represents the signal from a single peptide. FIG. 11C: TNFR1 binding site peptides illustrated by decrease in binding intensity when the TNFA/TNFR1 complex.

FIGS. 12A-12C. Synbody library production. FIG. 12A: Reaction scheme to produce synbody library. FIG. 12B: Pairwise combinations of peptides to produce 55 synbodies from 45 reactions. FIG. 12C: Average amount of hetero-bivalent and homo-bivalent synbody recovered from synbody library production for a protein target.

FIGS. 13A-13B. Multivalent structures for trapping engineered T cells. FIG. 13A: Branched DNA dendrimers (4). FIG. 13B: repeating DNA ribbons attached to a targeting nanostructure allows for a single recognition event to coat a tumor with multiple copies of a ssDNA handle. Engineered T cells modified with similarly multivalent structures are trapped at the tumor site upon circulation.

FIGS. 14A-14C. Some embodiments of a DNA nanocage of this disclosure. FIG. 14A: a tubular DNA nanocage of this disclosure. FIG. 14B: A tetrahedral DNA nanocage. FIG. 14C: A pyramidal DNA nanocage.

FIGS. 15A-15C. Chemokine expression levels in MDA-MB-231 breast cancer cells. Immunohistochemistry Staining (IHC) staining of chemokines in MDA-MB-231-derived tumor. FIG. 15A: high level chemokines, FIG. 15B: Low level chemokines FIG. 15C: Medium level chemokines

FIGS. 16A-16B. Nucleolin aptamer-functionalized DNA nanocage. (FIG. 16A) Schematic illustration of the construction of a thrombin-loaded DNA nanocage that opens into a flat sheet in response to nucleolin binding. (FIG. 16B) Corresponding AFM images.

FIG. 17. Depiction of strategies to enhance engineered T cell functions. DNA origami carrying matrix metallopeptidases (MMPs) or heparinase (HSPE) help engineered T cells degrade extracellular matrix (ECM) of cancer. Peptide or aptamer block inhibitory cytokine pathways of engineered T cells. Increased engineered T cell survival rate is achieved by cytokines linked to the engineered T cell surface.

FIG. 18. Design of lentiviral vector encoding two adaptor-CAR. A SNAP-Tag™ with CAR structure following with a CLIP-Tag™, different linkers and a transmembrane domain (TM) and luciferase (Luc).

FIG. 19. Inhibition ELISA to screen inhibitors for PD-1/PD-L1 inhibition. PD-1 is immobilized in the wells of a 96-well plate and 1.25 ug/mL PD-L1-Fc is incubated with and without inhibitors. Non-inhibiting synbodies (squares) and a known PD-L1 inhibitor (round circles) were screened.

FIGS. 20A-20C. Cytokines increase T cell proliferation and CCR8 mRNA expression. (FIG. 20A) Chemokine receptor mRNA expression levels in antigen-specific T cells. (FIG. 20B) Cytokine-induced CCR8 expression in antigen-specific T cells. (FIG. 20C) Cytokine increased T cell proliferation in antigen-specific T cells.

FIGS. 21A-21F. Depiction of bioconjugation strategies. (FIG. 21A) Copper-free click chemistry for attaching a synbody to a DNA handle. (FIG. 21B) MALDI-TOF MS of a purified peptide-DNA conjugate. (FIG. 21C) Using DNA to template the two peptides of a synbody pair. (FIG. 21D) Structure of IL-2 bound with its receptor (left) and MMP-2 indicating the catalytic site (right). The indicated interfaces are modified to avoid compromising protein function. (FIG. 21E) Cysteine alkylation using maleimide-DNA to modify the protein surface. (FIG. 21F) Sortase-mediated conjugation of oligonucleotide handles using a peptide-DNA conjugate. FIG. 21F discloses SEQ ID NO: 12.

FIG. 22. The schematics show the strategy for economic production of DNA nanostructures.

FIG. 23. Tracking adoptive transfer T cells by bioluminescence. T cells expressing luciferase were injected into mice through subcutaneous (Sub-Q, middle) or intravenous (I.V., right). Photos were taken at day 7 after T cell injections.

FIG. 24. In vivo stability and delivery of dual labeled DNA (red) and SA (green) to the neck lymph nodes (highlighted in circles) at various times after injections of either the mixture of DNA and SA or DNA-TH assembled SA.

FIGS. 25A-25H. Thrombin comprising DNA nanocage for on-target tumor infection in vivo. (FIG. 25A) Schematic representation of the therapeutic mechanism of nanocage within tumor vessels. DNA nanocages were administrated to breast tumor xenografted mice intravenously and were targeted to the tumor-associated vessels. The nanocages binds to the vascular endothelium by recognizing nucleolin and opens to expose the encapsulated thrombin, which induces a localized thrombosis, tumor infarction and cell necrosis. (FIG. 25B) Optical imaging of a MDA-MB-231 human breast tumor-comprising mouse before and after intravenous injection of Cy5.5-labeled nanocages. A high-intensity fluorescence signal was detected only in the tumor region of mice 8 h post-injection. (0 h=before injection.) (FIG. 25C) In vivo fluorescence intensity at the tumor sites was quantified as Total Fluorescence Intensity (TFI) at several time points after administration of the nanorobots; n=3. (FIG. 25D) FITC-labeled nanocages were injected intravenously into mice comprising MDA-MB-231 tumors. Tumors were harvested 8 h later, and tumor sections were stained with antibodies and examined by confocal microscopy. The nanocages (green) appear in the blood vessel-rich regions (anti-CD34; brown). Nuclei were stained with DAPI (blue). Scale bars, 20 μm. (FIG. 25E) Immunostaining to detect thrombosis (brown) in MDA-MB-231 tumors before and 24, 48 or 72 h post-administration of nanorobot-Th. (FIG. 25F) Haematoxylin and eosin (H&E) staining of the corresponding sections reveals a widespread necrosis (N). Scale bars, 50 μm. Data are representative of at least three separate experiments. (G&H) MDA-MB-231 tumor-comprising mice were treated on day 0 with saline, free thrombin (˜1.5 U/mouse), empty nanocages or nanocage-Th. Tumor volumes up to day 21 (FIG. 25G) and cumulative survival of mice (FIG. 25H) were shown. *p<0.05, **p<0.01 and ***p<0.001, compared with the other groups.

FIGS. 26A-26D. Anti-tumor activity and safety of DNA nanocage. (FIG. 26A) Inhibition of tumor growth by thrombin-loaded DNA nanocage; (FIG. 26B) Reduction of tumor loads in the liver of the mice treated with thrombin-loaded DNA nanocage; (FIG. 26C) Serum cytokine levels and (FIG. 26D) Microthrombi in cerebral venous vessels were detected at various time points post treatment.

FIG. 27. Combinatory effect of DNA-TH-CpG and anti-PD1 antibody on tumor suppression. A tumor was formed 10 days post sub-Q injection of A20-iRFP cells. Different groups of tumor-comprising mice were treated with intra-tumor injection of 1) DNA-TH-CpG followed by anti-PD1 antibodies on both day 2 and day 4; 2); DNA-TH-CpG (Day0) and 3) anti-PD1 antibodies. Tumor growth was monitored with LI-COR Pearl Small Animal Imaging system, and the images were linked and quantified with ImageStudio software.

FIG. 28. The strategy for CAR T cell therapy. A viral vector is transfected into a T cell to produce a CAR construct expressed on or about the surface of a T cell. The scFv of the CAR binds to its cognate ligand (e.g., CD19), bringing the T cell into local proximity with the tumor cell and activating the T cell to release cytolytic activity including cytokine release and T cell proliferation. In some embodiments, the engineered T cells of this disclosure can comprise a targeting molecule and/or agent which binds to a cognate ligand resulting cytolytic activity including cytokine release and T cell proliferation.

FIG. 29A depicts the autologous T cell isolation and activation strategy of T cells from a subject, engineering said T cells, and administering the engineered T cells into the subject. FIG. 29B shows optional CAR constructs of this disclosure. FIG. 29C shows optional targeting agent constructs of this disclosure. FIG. 29D shows a summary of the number and status of Clinical Trials using DNA-CAR T cells in cancer immunotherapy.

FIGS. 30A-30B. Clinical outcome of published CAR T therapies. FIG. 30A shows the present clinical outcomes of other attempts at published CAR T cell therapy for hematological malignancies, indicating that T cells targeted to CD19 have the highest rate of complete response or positive response for hematological malignancies. FIG. 30B shows the present clinical outcomes of other attempts at published CAR T therapy for solid tumor therapies, indicating that T cells targeting GD2 have the highest complete response or positive response for solid tumor malignancies.

FIG. 31. Summary of challenges of published CAR T cells in solid tumor therapy which demonstrate the difficulties of targeting solid tumor cells with a T cell therapy. The challenges include low pH/oxygen and nutrients, poor antigen presentation, cancer associated fibroblasts, ECM, inhibitory protein expression, suppressive cells, suppressive cytokines and chemokines. Each of the challenges and difficulties are overcome using the novel methods of the present disclosure as shown in FIG. 31B.

FIG. 32. Depiction one embodiment of a CAR structure which comprises a targeting agent (“targeting element”) (e.g., a scFv), a spacer, a transmembrane domain, a costimulatory domain, and a signaling domain. The costimulatory domain and signaling domain are positioned to be intracellular, while the targeting element is extracellular. FIG. 32 depicts a CAR comprising an adaptor protein which is connected to a first oligonucleotide, but where the first oligonucleotide is connected to a CLIP-Tag™ protein which is tethered to a Langmuir-Blodgett film for downstream T cell signaling analysis. The CAR in FIG. 32 also lacks a spacer domain. In some embodiments, the engineered CAR does not include the CAR depicted in FIG. 32.

FIGS. 33A-33B. FIG. 33A depicts a “Third generation CAR T cell” (e.g., CD19 scFv-comprising T cell), and its disadvantages in T cell therapy which include only targeting a single target is limited to addressing only one challenge category of solid tumor cell targeting, and reliance on genetic engineering to optimize binding affinity. FIG. 33B shows the strategy for designing some embodiments of engineered T cells of this disclosure by including a targeting agent which comprises a DNA nanostructure which, in some embodiments, further comprises a plurality of targeting molecules. The advantages of an engineered T cell include the ability to address multiple targets, the ability to address multiple challenges in targeting solid tumors, and the ease of assembly to modulate different targets. Also depicted is an AFM image of a DNA nanostructure with selectively positioned targeting molecules demonstrating that the targeting molecules are precisely positioned relative to each other to enhance both binding affinity and binding avidity.

FIG. 34. Depiction of the general adoptive transfer strategy utilizing a variety of engineered T cell types based on a variety of engineered T cells of this disclosure.

FIG. 35. Depiction of the proof of concept experiments performed in this disclosure to establish the enablement of the methods described herein. Luciferase levels are measured when the T cells are activated upon a stimulus in the model Luc-infected Jurkat cells. In addition, the cells are transfected with a GFP gene for co-location of the cells when performing fluorescent imaging.

FIG. 36. MALDI-TOF confirmation of the synthesis of one embodiment of an oligonucleotide modified with a protein tag (e.g., benzoguanine, “BG”).

FIG. 37. One embodiment of the transfection strategy for incorporating a vector encoding the engineered CAR into T cells. The first step is to generate a T cell line expressing engineered CAR by lentivirus transduction.

FIG. 38. Engineered CAR-GFP transduced T cells and wildtype (WT) T cells are imaged by fluorescent microscopy demonstrate that the transduced cells express GFP. In some embodiments, the transduced GFP cells are isolated and expanded for future experiments and/or T cell treatment therapy.

FIG. 39A: Luciferase activity levels (indicating T cell activation levels) of MDA-MB-231 breast cells and anti-PD-L1 engineered T cells incubated for 12 hours showing that anti-PD-L1 engineered T cells can be activated by MDA-MB-231 cells. FIG. 39B: Luciferase activity levels (indicating T cell activation levels) of A549 lung cancer cells and anti-EGFR engineered T cells incubated for 12 hours showing that anti-EGFR engineered T cells can be activated by A549 tumor cells. FIG. 39C: Luciferase activity levels (indicating T cell activation levels) of MUC1-1 engineered T cells incubated with MCF7 tumor cells showing that anti-MUC1-1 engineered T cells can be activated by MCF7 tumor cells. FIG. 39D: Luciferase activity levels (indicating T cell activation levels) of MUC1-2 engineered T cells incubated with MCF7 tumor cells showing that anti-MUC1-2 engineered T cells can be activated by MCF7 tumor cells. FIG. 39E: Luciferase activity levels (indicating T cell activation levels) of MUC1-3 engineered T cells incubated with MCF7 tumor cells showing that anti-MUC1-2 engineered T cells can be activated by MCF7 tumor cells.

FIG. 40: Representative aptamers of the present disclosure.

FIG. 41A: Engineered CAR design. FIG. 41B: Targeting molecules of this disclosure which are chemically conjugated to an oligonucleotide. FIG. 41C: Mechanism by which engineered T cells (“DNA-CAR T cell”) interact with tumor cell and release cytolytic enzymes.

FIG. 42A: Lentivirus transfer plasmid encoding an engineered CAR. FIG. 42B: Procedure of transferring lentiviral vector into Jurkat T cells. FIG. 42C: GFP fluorescence intensity of flow cytometry sorted cells shows that the adaptor-infected Jurkat T cells encode the engineered CAR.

FIG. 43A: Adaptor Jurkat T cells (engineered T cells) conjugate BG ss DNA (polyT) and hybridize with a complement oligonucleotide (Alexa647-polyA). FIG. 43B: Fluorescence microscopy image of adaptor Jurkat T cells linked to a molecule through DNA hybridization. FIG. 43C: Fluorescence intensity of Alexa647 on engineered T cells shows that the cells with adaptor protein is higher than cells without adaptor structures. FIG. 43D: The ratio of Alexa 647 positive engineered T cells to all cells (GFP cells) shows a dose-dependent signal as the BG-polyT concentration added to the cells is increased.

FIG. 44A: Adaptor Jurkat T cell conjugation to BG-polyT to generate polyT engineered T cells. FIG. 44B: Monitoring T cell activation of released luciferase in the presence of polyA-coated agarose beads shows the T cells are activated in the presence of a model target.

FIG. 45A: Procedure of conjugating fluorescent aptamers to engineered T cells. FIG. 45B: Fluorescence intensity of Alexa488-labelled scg8 aptamers in the presence of CCRF-CEM cancer cells showing that the dye labelled aptamer can recognize the CCRF-CEM successfully.

FIG. 46A-FIG. 46B: Activation of scg8 aptamer-comprising T cells by CCRF-CEM cancer cells. FIG. 46A: Adaptor Jurkat T cells conjugate with BG-polyT to generate polyT comprising engineered T cells. FIG. 46B: Luciferase assay measurements showing that engineered T cells are activated by target cancer cells.

FIG. 47: Relative dimensions of engineered T cell to CCRF-CEM cancer cell depicting the involved components of the cell-cell interaction.

FIG. 48: Mice model dosing and monitoring regimen.

FIG. 49: CD19 CAR with linkers help T cell release more IFN-gamma when cosulcutre with Raji cells. The length of the linker affects the T cell activity.

FIG. 50A: Production of PD-L1 engineered T cells. FIG. 50B: SDS-PAGE of PD-L1 antibody (α-PD-L1) and PD-L1 antibody conjugated with polyT (Poly T α-PD-L1). FIG. 50C: Luciferase assay results showing PD-L1 positive MDA-MB-231 cells activate PD-L1 DNA-CAR T cells.

FIG. 51 The plasmid construct of a lentivirus encoding one embodiment of an engineered CAR of this disclosure.

FIG. 52 The DNA sequence inserted into a lentivirus encoding one embodiment of an engineered CAR of this disclosure.

FIG. 53 lists representative staple strands of this disclosure.

FIG. 54A: DNA origami-assisted CD8+ T cells targeting and killing of leukemia cells. FIG. 54B: Scheme and principle of cell-killing assay at different effector to target ratios. FIG. 54C: Fluorescence image overlays of primary CD8+ T cells and their interaction with L1210 cells. Scale bars: 10 nm. FIG. 54D: Cell survival of target cell at different effector to target (E: T) ratio.

### DETAILED DESCRIPTION

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their System International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

To overcome obstacles including barriers, loss of antigen and immunosuppressive environments, different strategies have been developed for CAR T cells, including CAR structures targeting two antigens to overcome antigen heterogeneity, engineering CARs to co-express chemokine receptors that increase T cell traffic to tumor area, and employing CARs that deplete the fibroblast cells that surround a solid tumor. However, T cells have only been engineerable to solve one of the above problems at a time. Furthermore, the foregoing strategies require long-term molecular cloning and structure optimizations which is an extremely costly and time-consuming process that is not always successful.

The inventors have recognized that the ability to manipulate T cells according to the methods disclosed herein provides a plethora of opportunities and advantages for additional changes and improvements.

In some embodiments, targeting agents comprise targeting molecules. In some embodiments, targeting molecules can include or exclude aptamers, peptides, or small molecules. Targeting agents and/or targeting molecules are attached to the engineered T cells using a site-specific type of chemistry using protein-tag chemistry. In some embodiments of this disclosure, for this purpose, SNAP-Tag™ or CLIP-Tag™ proteins are incorporated into the CARs. In some embodiments, the targeting agents and molecules, include DNA nanostructures and proteins, and are conjugated with a single strand DNA sequence complimentary to a single stranded DNA oligonucleotide sequence connected to the engineered T cell surface.

In some embodiments, this disclosure provides a method to generate DNA, RNA and/or DNA-peptide nanostructure based chimeric antigen receptor (CAR) T cell (engineered T cell) for cancer immunotherapy. This method allows rapid generation of single- or multiple-target antigen-specific T cells, ECM degrading T cells (including matrix metalloproteinase comprising targeting agents), cytokine expressing T cells and inhibitory pathway overcoming T cells without further genetic engineering.

In some embodiments, this disclosure provides a novel DNA nanotechnology-based strategy to engineer T cells to: 1) Allow for precise tumor targeting through dual/multiple-binding specificities to minimize tumor immune escape; 2) Increase cell survival and trafficking to tumor area; and 3) Generate multifunctional engineered T cells rapidly economically. An engineered CAR T cell toolkit is generated using DNA nanotechnology (engineered T cell). T cells are engineered to express signaling CAR domains and an “adaptable receptor” protein domain that allows the attachment of a targeting agent with high efficiency. In some embodiments the targeting agent is a DNA nanostructure (also referred to herein as a “DNA nano-scaffold”). These DNA scaffolds carry targeting molecules which can include or exclude peptides, synbodies, aptamers, or small molecules, and functional molecules which enhance T cell survival penetration into the solid tumor environment. This highly modular and combinatorial approach circumvents the long, expensive process of specific genetic engineering, allow for non-genetically encodable functionality, and enhances T cell activity through multivalent effects. The engineered T cell is unprecedented in its economic manufacture and broad use in cancer immunotherapy research and clinical application. The engineered T cells comprising targeting agents which comprise DNA significantly improve the success of engineered T cells against solid tumors and are therefore highly beneficial for personalized cancer immunotherapy.

Using DNA, RNA and/or peptide nanotechnologies, engineered T cells are provided that more precisely target tumor cells with remarkable specificity for different levels of expression, have increased survival resistance to the immunosuppressive solid tumor environment, and increased T cell traffic to the tumor area.

The inventors have recognized that the designed engineered T cells addresses the problems of T cell immunotherapy for solid tumor cancers of a low level or loss of tumor antigen expression, poor T cell trafficking and infiltration into the tumor, and an immune response-repressive environment.

### Definitions

Abbreviations: CAR: chimeric antigen receptor; ACT: adoptive T cell transfer; TAA tumor-associated antigens; CTL: cytolytic lymphocytes; DCs: dendritic cells; scFv: single-chain variable fragment; ELISPOT: enzyme-linked immunosorbent spot; TME: tumor microenvironment; PD-1: programmed cell death protein 1; GFP: green fluorescence protein; CTLA4: cytotoxic T-lymphocyte-associated antigen 4; MDSC: myeloid derived suppressor cell; MHC-II: major histocompatibility complex-II; Tregs: regulatory T cells; OVA: ovalbumin; ECM: extracellular matrix; MMP: matrix metallopeptidase; RLU: relative light unit; SA: streptavidin; siRNA: small interfering RNA; Synbody: peptide-based multivalent synthetic antibodies; SELEX: systemic evolution of ligands by exponential enrichment; SPR: surface plasmon resonance; IHC: immunohistochemistry; MSLN: mesothelin, CEA: carcinoembryonic antigen; EGFR: Epidermal growth factor receptor, GPC3: Glypican-3; HER2: human epidermal growth factor receptor 2; nt: nucleotides (often used to refer to the number of nucleotides in a ssDNA strand); bp: basepairs (often used to refer to the number of nucleotides in a ssDNA strand).

As used herein, the term “activation” refers to the state of an immune cell, e.g., a T cell, that has been sufficiently stimulated to induce detectable cellular proliferation. Activation can also be associated with induced cytokine production and detectable effector functions. The term “activated T cells” refers to, among other things, T cells that are undergoing cell division.

As used herein, the term “administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

As used herein, the term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions are further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” can include or exclude both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. The term “antibody” also includes an antigen-binding fragment or an antigen-binding portion of any of the aforementioned immunoglobulins, and includes a monovalent and a divalent fragment or portion, and a single chain Ab.

As used herein, the term “antigen binding molecule” or “antibody fragment” refers to any portion of an antibody less than the whole. An antigen binding molecule can include the antigenic complementarity determining regions (CDRs). Examples of antibody fragments can include or exclude, Fab, Fab′, F(ab′)2, and Fv fragments, dAb, linear antibodies, scFv, nanobodies, and multispecific antibodies formed from antigen binding molecules.

As used herein, the term “antigen” refers to any molecule that provokes an immune response or is capable of being bound by an antibody. The immune response may involve either antibody production, or the activation of specific immunologically-competent cells, or both. A person of skill in the art would readily understand that any macromolecule, including virtually all proteins or peptides, can serve as an antigen. An antigen can be endogenously expressed, i.e., expressed by genomic DNA, or can be recombinantly expressed. An antigen can be specific to a certain tissue, including a cancer cell, or it can be broadly expressed. In addition, fragments of larger molecules can act as antigens. In one embodiment, antigens are tumor antigens.

As used herein, the term “epitope” refers to the portion of an antigen capable of eliciting an immune response, or the portion of an antigen that binds to an antibody. Epitopes can be a protein sequence or subsequence that is recognized by an antibody.

As used herein, the term “single chain antibody” (scFv) refers to an immunoglobulin molecule with function in antigen-binding activities. An antibody in scFv (single chain fragment variable) format consists of variable regions of heavy (VH) and light (VL) chains, which are joined together by a flexible peptide linker.

As used herein, the term “synbody” refers to a synthetic molecule having a molecular weight of about 6 kDa to about 8 kDa and comprise bivalent peptides that bind their target with antibody-like affinity. The synbody mimics a synthetic antibody. The two peptides are different and bind to orthogonal sections of the target. The two peptides are conjoined either directly through a linker or indirectly. The directly conjoined peptides linked through a linker are linked using a trivalent linker as described herein. The indirectly conjoined peptides are brought in local proximity when a first peptide is connected to a first oligonucleotide, and a second peptide is connected to a second oligonucleotide, and a portion of the first oligonucleotide sequence is complementary to a portion of the second oligonucleotide sequence such that the first and second oligonucleotides form a hybridization complex.

As used herein, the term “autologous” refers to any material derived from the same individual to which it is later to be re-introduced. In some embodiments, the engineered autologous T cell therapy method described herein involves collection of lymphocytes from a patient, which are then engineered to express, e.g., an engineered CAR construct, contacted with a protein tag which is connected with ta first oligonucleotide, contacted with a targeting agent which is connected to a second oligonucleotide, wherein a portion of the first oligonucleotide and a portion of the second oligonucleotide are complementary, and then administered back to the same patient.

As used herein, the term “allogeneic” refers to any material derived from one individual which is then introduced, after T cell engineering according to the methods described herein, to another individual of the same species, e.g., allogeneic engineered T cell transplantation. As used herein, the term “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. In some embodiments, a “cancer” or “cancer tissue” can include a tumor. The types of cancers that are treated by the methods of this disclosure can include or exclude cancers of the immune system including lymphoma, acute lymphoblastoid leukemia, leukemia, and other leukocyte malignancies. In some embodiments, the methods of this disclosure reduce the tumor size of a tumor derived from, for example, bone cancer, pancreatic cancer, breast cancer, brain cancer, lung cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, and combinations of said cancers. In some embodiments, the cancer is acute lymphoblastoid leukemia.

As used herein, the term “anti-tumor effect” refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor.

As used herein, the term “progression-free survival,” or “PFS,” refers to the time from the treatment date to the date of disease progression per the revised IWG Response Criteria for Malignant Lymphoma or death from any cause.

As used herein, the term “complementary” refers to the concept of subunit sequence complementarity between two nucleic acids, e.g., two DNA molecules, or two RNA molecules, or a DNA molecule with a cognate RNA molecule. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A: T and G: C nucleotide pairs for DNA, or A: U and G: C for RNA).

An antisense sequence refers to a DNA sequence which is complementary to the sequence of a DNA molecule encoding a protein. The antisense sequence does not need to be complementary to the coding portion of the coding strand of the DNA molecule only.

As used herein, the term “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

As used herein, the term “encoding” refers to the innate property of specific sequences of nucleotides in a polynucleotide, including a gene, a cDNA, or an mRNA, to serve as templates for the synthesis of other biopolymers having either a defined sequence of nucleotides or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and the non-coding strand, used as the template for transcription of a gene or DNA, can be referred to as encoding the protein or other product of that gene.

As used herein, the term “cytokine,” refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include or exclude homeostatic cytokines, chemokines, pro-inflammatory cytokines, effectors, and acute-phase proteins. Homeostatic cytokines, including interleukin (IL) 7 and IL-15, promote immune cell survival and proliferation, and pro-inflammatory cytokines can promote an inflammatory response. Examples of homeostatic cytokines can include or exclude, IL-2, IL-4, IL-5, IL-7, IL-10, IL-12p40, IL-12p70, IL-15, and interferon (IFN) gamma. Examples of pro-inflammatory cytokines can include or exclude, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors can include or exclude, granzyme A, granzyme B, soluble Fas ligand (sFasL), and perforin. In some embodiments, acute phase-proteins can include or exclude C-reactive protein (CRP) and serum amyloid A (SAA). In some embodiments, cytokines of this disclosure can include or exclude: chemokine (C-C motif) ligand (CCL) 1, CCL5, monocyte-specific chemokine 3 (MCP3 or CCL7), monocyte chemoattractant protein 2 (MCP-2 or CCL8), CCL13, IL-1, IL-3, IL-9, IL-11, IL-12, IL-14, IL-17, IL-20, IL-21, granulocyte colony-stimulating factor (G-CSF), leukemia inhibitory factor (LIF), oncostatin M (OSM), CD154, lymphotoxin (LT) beta, 4-1BB ligand (4-1BBL), a proliferation-inducing ligand (APRIL), CD70, CD153, CD178, glucocorticoid-induced TNFR-related ligand (GITRL), tumor necrosis factor superfamily member 14 (TNFSF14), OX40L, TNF- and ApoL-related leukocyte-expressed ligand 1 (TALL-1), or TNF-related apoptosis-inducing ligand (TRAIL).

As used herein, the term “chemokines” refers to a type of cytokine that mediates cell chemotaxis, or directional movement. Examples of chemokines can include or exclude, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

As used herein, the terms “serum level” and “serum concentration” refer to the amount of an analyte in the serum of a subject. Serum levels of a given analyte are measured using any method known in the art. For example, cytokine serum levels are measured using an enzyme-linked immunosorbent assay (ELISA). In one particular embodiment, cytokine serum levels are measured using an EMDmillipore LUMINEX® xMAP® multiplex assay.

As used herein, the term “dosing interval,” refers to the amount of time that elapses between multiple doses of a formulation disclosed herein being administered to a subject. Dosing interval can thus be indicated as ranges.

Doses can be presented as a “weight based dose” or as a “body surface area (BSA) based dose.” A weight based dose is a dose that is administered to a patient that is calculated based on the weight of the patient, e.g., mg/kg. A BSA based dose is a dose that is administered to a patient that is calculated based on the surface area of the patient, e.g., mg/m². The two forms of dose measurement can be converted for human dosing by multiplying the weight based dose by 37 or dividing the BSA based dose by 37. For example, a dose of 60 mg/kg to be administered to a human subject is equivalent to a 2220 mg/m² dose of the same drug to be administered to the same subject.

As used herein, the term “dosing frequency” refers to the frequency of administering doses of a formulation disclosed herein in a given time. Dosing frequency can be indicated as the number of doses per a given time.

As used herein, the term “fragment” as applied to a polynucleotide sequence, refers to a portion of said polynucleotide sequence which consists essentially of less than the entire polynucleotide sequence. In some embodiments, the polynucleotide fragment is at least about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 200, 300, 400, or 500 nucleotides in length, or any number of nucleotides in between any of the aforementioned values.

As used herein, the term “genomic DNA” refers to a DNA strand which has a nucleotide sequence homologous with a gene as it exists in the natural host.

As used herein, the term “homologous” or “identity” refers to the subunit sequence similarity between two polymeric molecules, e.g., between two nucleic acid molecules, e.g., two DNA molecules or two RNA molecules, or between two polypeptide molecules. When the terms “homology” or “identity” are used herein to refer to the nucleic acids and proteins, it should be construed to be applied to homology or identity at both the nucleic acid and the amino acid sequence levels.

As used herein, the following abbreviations for the commonly occurring nucleic acid bases are used: “A” refers to adenosine, “C” refers to cytidine, “G” refers to guanosine, “T” refers to thymidine, and “U” refers to uridine.

As used herein, when describing two polynucleotides (also referred to as “polynucleotide sequence”) as “operably linked”, a single-stranded or double-stranded nucleic acid moiety comprises the two polynucleotides arranged within the nucleic acid moiety in such a manner that at least one of the two polynucleotides is able to exert a physiological effect by which it is characterized upon the other. In some embodiments, a promoter polynucleotide sequence operably linked to the coding region of a gene promotes transcription of the coding region.

As used herein, when describing an oligonucleotide as “connected” to another moiety, the oligonucleotide is covalently bound to said moiety. The covalent bond is formed using a linker molecule (e.g., sulfo-SMCC (ThermoFisher), where the oligonucleotide is 5′ or 3′ modified with the appropriate functional group (amino, sulfuryl, carboxyl, aldehyde, etc.—all available from Glen Research Inc.) to bind to the linker. The moiety can likewise be functionalized, or possess an innate functional group (e.g., amino group from a lysine amino acid in a protein) to react to said linker group.

When the nucleic acid encoding the desired protein further comprises a promoter/regulatory sequence, the promoter/regulatory is positioned at the 5′ end of the desired protein coding sequence such that it drives expression of the desired protein in a cell.

As used herein, the term “promoter sequence” refers to a nucleic acid sequence which is required for expression of a gene product operably linked to the promoter/regulatory sequence. In some embodiments, this conjoined sequence may be the core promoter sequence. In other embodiments, this conjoined sequence may also comprise an enhancer sequence and other regulatory elements which are required for expression of the gene product. In some embodiments, the promoter/regulatory sequence may be one which expresses the gene product in a tissue specific manner.

As used herein, the term “polynucleotide” or “polynucleotide sequence” refers to a single strand or parallel and anti-parallel strands of a nucleic acid. The polynucleotide may be either a single-stranded or a double-stranded nucleic acid.

As used herein “nucleic acid,” refers to any compound and/or substance that comprise a polymer of nucleotides linked via a phosphodiester bond. Exemplary nucleic acids include ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or hybrids thereof. They may also include RNAi-inducing agents, RNAi agents, siRNAs, shRNAs, miRNAs, antisense RNAs, ribozymes, catalytic DNA, RNA, RNAs that induce triple helix formation, aptamers, vectors, etc.

In some embodiments, modified nucleosides and nucleotides are prepared according to the synthetic methods described in Ogata et al. Journal of Organic Chemistry 74:2585-2588, 2009; Purmal et al. Nucleic Acids Research 22(1): 72-78, 1994; Fukuhara et al. Biochemistry 1(4): 563-568, 1962; and Xu et al. Tetrahedron 48(9): 1729-1740, 1992, each of which are incorporated by reference.

Modified nucleic acids (e.g., staple strands chemically connected or conjugated to a targeting molecule) need not be uniformly modified along the entire length of the molecule. Different nucleotide modifications and/or backbone structures may exist at various positions in the nucleic acid. The nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, including at least 50% modified nucleotides, at least 80% modified nucleotides, or at least 90% modified nucleotides.

In some embodiments, a staple strand is a modified nucleic acid.

The synthetic modified oligonucleotides described herein include modifications to prevent rapid degradation by endo- and exo-nucleases and to avoid or reduce the cell's innate immune or interferon response to the oligonucleotide. Modifications can include or exclude, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

As used herein, the term “central polynucleotide strand” refers to a polynucleotide sequence which is about 45 to about 15,000 nucleotides in length, and can form tertiary structures via Watson-Crick basepairing and/or intra-duplex interactions.

As used herein, “staple strands” are short single-stranded oligonucleotides of about 20 to about 40 nucleotides in length, including 20, 21, 22, 23, 24, 25, 26, 27, 28, 29, 30, 31, 32, 33, 34, 35, 36, 37, 38, 39, or 40 nucleotides in length, wherein one end of the staple strand hybridizes with a region of the scaffold strand, and the second end of the staple strand hybridizes with another region of the scaffold strand, thereby “stapling” the two regions of the scaffold strand. Exemplary staple strands include those described herein and in the included Sequence Listing.

As used herein, the term “adaptor protein” or “adaptor receptor” refers to a protein which can form covalent bonds to a specific predetermined small molecule which is covalently connected to an oligonucleotide. In some embodiments, the adaptor protein is selected from SNAP-Tag™, CLIP-Tag™, or HALO-Tag™.

As used herein, the term “engineered T cells” or “engineered CAR T cells” refers to T cells comprising signaling CAR domains and further comprising an “adaptor protein” that enables the attachment of a targeting agent via immobilization to a first oligonucleotide chemically conjugated to a targeting agent and/or targeting molecule. In some embodiments, the targeting agent comprises a DNA origami nano-scaffold (“DNA nanostructure”).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.”

As used herein, the term “portion” of a polynucleotide refers at least at least about twelve sequential nucleotide residues of the polynucleotide. It is understood that a portion of a polynucleotide may include every nucleotide residue of the polynucleotide.

As used herein, the term “recombinant polynucleotide” refers to a polynucleotide having sequences that are not naturally joined together. An amplified or assembled recombinant polynucleotide may be included in a suitable vector, and the vector transform a suitable host cell. In some embodiments, a recombinant polynucleotide may serve a non-coding function (e.g., promoter, origin of replication, spacer, ribosome-binding site, etc.) as well.

As used herein, the term “recombinant polypeptide” refers to a polypeptide which is produced upon expression of a recombinant polynucleotide.

As used herein, the term “polypeptide” refers to a biopolymer consisting essentially of amino acid residues, related naturally occurring structural variants, and synthetic non-naturally occurring analogs thereof linked via peptide bonds, related naturally occurring structural variants, and synthetic non-naturally occurring analogs thereof. A protein or polypeptide must contain at least two amino acids, and no limitation is placed on the maximum number of amino acids that can comprise a protein's or peptide's sequence. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

Conventional notation is used herein to portray polypeptide sequences: the left-hand end of a polypeptide sequence is the amino-terminus; the right-hand end of a polypeptide sequence is the carboxyl-terminus.

As used herein, the term “fluorescent protein domain” refers to a polypeptide, the expression of which can be detected using a known method. In some embodiments, the fluorescent protein domain is GFP.

As used herein, the term “high stringency” refers to when a first oligonucleotide anneals with a second oligonucleotide under conditions whereby only oligonucleotides which are at least about 73%, more preferably, at least about 75%, even more preferably, at least about 80%, even more preferably, at least about 85%, yet more preferably, at least about 90%, and most preferably, at least about 95%, complementary anneal with one another. The stringency of conditions used to anneal two oligonucleotides is a function of, among other factors, ionic strength of the annealing medium, temperature, the incubation period, the length of the oligonucleotides, the G-C content of the oligonucleotides, and the expected degree of non-homology between the two oligonucleotides.

As used herein when referring to polypeptides, the term “protein tag” or “polypeptide tag” refers to any chemical moiety which, when linked by a peptide bond to a protein of interest, may be used to form a covalent bond with the binding site of an adaptor protein. In some embodiments, the polypeptide tag can be selected from a SNAP-Tag™, CLIP-Tag™, or HALO-Tag™ tag. The SNAP-Tag™ is an approximately 20 kDa version of a protein O′-alkylguanine-DNA alkyltransferase which has a single reactive cysteine with a very high affinity for guanines alkylated at the O′-position. The alkyl group, including any immobilization moiety attached to the alkyl group (e.g., an oligonucleotide connected to the guanine via a linker (e.g., sulfo-SMCC) can be transferred covalently from the guanine to the cysteine in the alkyltransferase protein. A CLIP-Tag™ is a modified version of SNAP-Tag™, engineered to react with benzylcytosine rather than benzylguanine derivatives. SNAP-Tag™ and CLIP-Tag™ are available from New England Biolabs.

The HALO-Tag™ is a 297 residue peptide (73.6 kDa) derived from a haloalkane dehalogenase and reacts with an alpha-halogenated haloalkane substituent to form a covalent bond. The HALO-Tag™ is available from Promega.

As used herein, the term “vector” refers to any plasmid or virus encoding an exogenous nucleic acid. The term should also be construed to include non-plasmid and non-viral compounds which facilitate transfer of nucleic acid into virions or cells, including, for example, polylysine compounds and the like. The vector may be a viral vector which is suitable as a delivery vehicle for delivery of a nucleic acid that encodes a CAR or the vector may be a non-viral vector which is suitable for the same purpose.

Examples of viral and non-viral vectors for delivery of DNA which encodes a CAR to cells and tissues are described in Ma et al. (1997, Proc. Natl. Acad. Sci. U.S.A. 94:12744-12746), herein incorporated by reference. Examples of viral vectors can include or exclude a lentiviral vector, a recombinant adenovirus, a recombinant retrovirus, a recombinant adeno-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994), each of which are herein incorporated by reference.

The term “stimulation,” as used herein, refers to a primary response induced by binding of a stimulatory molecule with its cognate ligand, wherein the binding mediates a signal transduction event. In some embodiments, when the first oligonucleotide connected to the protein tag is hybridized to a second oligonucleotide, stimulation of the primary T cell begins, thereby mediating a primary response by the T cell, including, but not limited to, activation, initiation of an immune response, proliferation, and the like. The targeting agent can bind to a cognate ligand, resulting in stimulation of the engineered T cell. In some embodiments, the targeting agent comprises a targeting molecule. The targeting molecule can target a stimulatory ligand which can include or exclude an MHC Class I molecule loaded with a peptide, an anti-CD3 antibody, a superagonist anti-CD28 antibody, and a superagonist anti-CD2 antibody.

As used herein, the term “stimulatory ligand,” refers to a ligand that when present on an antigen presenting cell (e.g., a cancer cell) can specifically bind with a cognate binding partner on a T cell, thereby mediating a primary response by the T cell, including, but not limited to, activation, initiation of an immune response, proliferation, and the like. Stimulatory ligands can include or exclude, inter alia, an MHC Class I molecule loaded with a peptide, an anti-CD3 antibody, an anti-CD19 antibody, a superagonist anti-CD28 antibody, and a superagonist anti-CD2 antibody. In some embodiments, the stimulatory molecule is the targeting agent.

As used herein, the term “inducing an immune response” refers to the activation of an immune cell. Methods of measuring an immune response include using an assay as described in the Examples.

Certain embodiments of the invention also provide a method of treating a disease or disorder in a subject, comprising administering to the subject a therapeutically effective amount of an engineered T cell or a composition as described herein.

In certain embodiments, the therapeutic agent is a vasoconstrictor. In certain embodiments, the vasoconstrictor is selected from thrombin, prothrombin, rhThrombin, fragments thereof, and combinations thereof. In certain embodiments, the therapeutic agent is thrombin. In some embodiments, the thrombin can be human thrombin, bovine thrombin, or murine thrombin. In some embodiments, the thrombin can be thrombin alpha.

As used herein, the term “aptamer” refers to a nucleic acid sequence that interacts with a ligand under normal physiological conditions.

As used herein, the terms “chimeric antigen receptor” or “CAR” refer to a fusion protein comprising antigen recognition moieties and cell-activation elements. In some embodiments, the CAR comprises an extracellular domain, a transmembrane domain, and an intracellular domain, each of which are polypeptides encoded by a corresponding polynucleotide sequence. In certain embodiments, the CAR is designed to have two, three, four, or more costimulatory domains.

As used herein, the terms “CAR T-cell” or “CAR T-lymphocyte” refer to a T-cell containing or capable of producing a CAR polypeptide, regardless of actual expression level. A cell that is capable of expressing a CAR is a T-cell containing nucleic acid sequences for the expression of the CAR in the cell. As used herein, the term “engineered T cell” refers to a T cell expressing a CAR which comprises an extracellular protein tag and a spacer domain polypeptide.

As used herein, the terms “T-lymphocyte” or T-cell” or “T cell” refer to a hematopoietic cell that normally develops in the thymus. T-lymphocytes or T-cells can include or exclude, natural killer T cells, regulatory T cells, helper T cells, cytotoxic T cells, memory T cells, gamma delta T cells and mucosal invariant T cells. In some embodiments, the T cell is a Jurkat T cell.

As used herein, a “transmembrane sequence” refers to the polypeptide sequence between the extracellular sequence and the intracellular sequence. A portion of the transmembrane sequence exists within the cell membrane. In some embodiments, a transmembrane sequence comprises one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane sequence is associated with one of the other elements used in the engineered CAR. 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane sequence is associated with one of the other polypeptide sequences used in the engineered CAR.

In some embodiments, the transmembrane sequence is derived from a natural polypeptide, or may be artificially designed. Transmembrane sequences derived from a natural polypeptide can be obtained from any membrane-binding or transmembrane protein. In some embodiments, the transmembrane sequence of a T cell receptor a or β chain, a CD3ζ chain, CD28, CD38, CD45, CD4, CD5, CD8, CD9, CD16, CD22, CD33, CD37, CD64, CD80, CD86, CD134, CD137, ICOS, CD154, or a GITR can be used. An artificially designed transmembrane sequence is a polypeptide comprising hydrophobic residues including leucine and valine. In some embodiments, a triplet of phenylalanine, tryptophan and valine is found at each end of the synthetic transmembrane sequence. In some embodiments, a short oligopeptide linker or a polypeptide linker, (e.g., a linker having a length of 2 to 10 amino acids) is arranged between the transmembrane sequence and the intracellular sequence. In some embodiments, a linker sequence having a glycine-serine continuous sequence can be used.

In some embodiments, a spacer sequence can be arranged between the extracellular sequence and the transmembrane sequence, or between the intracellular element and the transmembrane element. In some embodiments, a spacer is an oligopeptide or polypeptide that serves to link the transmembrane element with the extracellular element and/or the transmembrane element with the intracellular element. In some embodiments, the spacer element comprises up to 300 amino acids, or 10 to 100 amino acids, or 25 to 50 amino acids. The spacer polypeptide sequence can be a spacer polypeptide domain. In some embodiments, the spacer polypeptide sequence can comprise glycine and serine amino acids in a repeating manner. In some embodiments, the spacer polypeptide sequence is (Gly-Gly-Gly-Gly-Ser) n, where n is an integer selected from 1 to 20 (SEQ ID NO: 3). In some embodiments, n is 4. In some embodiments, the spacer can include or exclude a hinge sequence. In some embodiments, the hinge sequence can be a mammalian CD8 hinge sequence. In some embodiments, the hinge sequence can be a human CD8 hinge sequence having the sequence comprising SEQ ID NO. 4: Thr Thr Thr Pro Ala Pro Arg Pro Pro Thr Pro Ala Pro Thr Ile Ala Ser Gln Pro Leu Ser Leu Arg Pro Glu Ala Cys Arg Pro Ala Ala Gly Gly Ala Val His Thr Arg Gly Leu Asp Phe Ala Cys Asp, or an amino acid sequence that has at least 95% sequence identity or 99% sequence identity to the amino acid sequence of SEQ ID NO. 4.

The chimeric antigen receptor can further comprise a hinge region. The hinge region can be derived from the hinge region of IgG1, IgG2, IgG3, IgG4, IgA, IgD, IgE, IgM, CD28, or CD8 alpha. In one particular embodiment, the hinge region is derived from the hinge region of human CD8 alpha.

The chimeric antigen receptor can also comprise a transmembrane domain. The transmembrane domain can be a transmembrane domain of any transmembrane molecule that is a co-receptor on immune cells or a transmembrane domain of a member of the immunoglobulin superfamily. In certain embodiments, the transmembrane domain is derived from a transmembrane domain of CD28, CD8 alpha, CD4, or CD19. In one particular embodiment, the transmembrane domain comprises a domain derived from a CD28 transmembrane domain.

The chimeric antigen receptor can further comprise one or more costimulatory signaling regions. For example, the costimulatory signaling region can be a signaling region of CD28, OX-40, 41BB, CD27, inducible T cell costimulator (ICOS), CD3 gamma, CD3 delta, CD3 epsilon, CD247, Ig alpha (CD79a), or Fc gamma receptor. In one particular embodiment, the costimulatory signaling region is a CD28 signaling region.

In one embodiment, the chimeric antigen receptor further comprises a CD3 zeta signaling domain.

The terms “serum level” and “serum concentration” are used interchangeably as used herein and refer to the amount of an analyte in the serum of a subject. Serum levels of a given analyte can be measured using any method known in the art. For example, cytokine serum levels are measured using an enzyme-linked immunosorbent assay (ELISA). In one particular embodiment, cytokine serum levels are measured using an EMDmillipore LUMINEX® xMAP® multiplex assay.

A “therapeutically effective amount,” “effective dose,” “effective amount,” or “therapeutically effective dosage” of an engineered T cell, drug or therapeutic agent is any amount of the cell or drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, including in human subjects during clinical trials, in animal model systems predictive of efficacy in humans, or by assaying the activity of the agent in in vitro assays. The term “effective amount,” in referring to adoptive cell transfer therapy using the engineered T cells described herein, includes an amount of an engineered T cell described herein that induces an immune response.

The term “lymphocyte” as used herein includes natural killer (NK) cells, T cells, or B cells. In some embodiments, the T-cells can include or exclude: Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory TSCM cells, like naive cells, are CD45RO−, CCR7+, CD45RA+, CD62L+ (L-selectin), CD27+, CD28+ and IL-7Ra+, but they also express large amounts of CD95, IL-2RB, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T-cells (Tregs, suppressor T cells, or CD4+CD25+ regulatory T cells), Natural Killer T-cells (NKT) and Gamma Delta T-cells. In some embodiments, the T-cell is a helper T-cell, a cytotoxic T-cell, a memory T-cell, a regulatory T-cell, a natural killer cell, a mucosal associated invariant T-cell, a gamma delta T cell, or a precursor or progenitor cell to the aforementioned. In some embodiments, the lymphocyte cell is a natural killer cell, or a precursor or progenitor cell to the natural killer cell.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which can either be obtained from a patient or a donor. The cell can be modified to express an exogenous construct, including, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy can include or exclude, T cell therapies. T cell therapy can include adoptive T cell therapy, tumor-infiltrating lymphocyte (TIL) immunotherapy, autologous cell therapy, engineered autologous cell therapy, and allogeneic T cell transplantation. Examples of T cell therapies for administration of T cells are described in U.S. Patent Publication Nos. 2014/0154228 and 2002/0006409, U.S. Pat. No. 5,728,388, and International Publication No. WO 2008/081035, incorporated by reference herein.

The T cells of the immunotherapy described herein can come from any source known in the art. For example, T cells are differentiated in vitro from a hematopoietic stem cell population, or T cells are obtained from a subject. In some embodiments, T cells can be obtained from peripheral blood mononuclear cells, bone marrow, lymph node tissue, cord blood, thymus tissue, tissue from a site of infection, ascites, pleural effusion, spleen tissue, and tumors. In some embodiments, the T cells are derived from one or more T cell lines available in the art. In some embodiments, T cells are obtained from a unit of blood collected from a subject using any number of techniques known to the skilled artisan, including FICOLL™ separation and/or apheresis. Methods of isolating T cells for a T cell therapy are disclosed in U.S. Patent Publication No. 2013/0287748, which is herein incorporated by reference.

The term “adoptive cell transfer,” as used herein, refers to a process by which a patient's own T cells are collected and subsequently genetically altered to recognize and target one or more antigens expressed on the cell surface of one or more specific tumor cells or malignancies. T cells are engineered to express, for example, engineered chimeric antigen receptors (CAR) to express an extracellular adaptor protein with specificity for a particular protein tag which is connected to an oligonucleotide.

A “patient” as used herein includes any human who is afflicted with a cancer (e.g., a lymphoma or a leukemia). The terms “subject” and “patient” are used interchangeably herein.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission.

As described herein, any concentration range, percentage range, ratio range or integer range is to be understood to include the value of any integer within the recited range and, when appropriate, fractions thereof (including one-tenth and one-hundredth of an integer), unless otherwise indicated.

Various aspects of the invention are described in further detail in the following subsections.

### Engineered DNA CAR

### Nucleic Acids

In some embodiments, this disclosure relates to the nucleic acids that encode, at least in part, the individual peptides, polypeptides, proteins, and RNA control devices of this disclosure. In some embodiments, the nucleic acids may be natural, synthetic or a combination thereof. The nucleic acids of the invention may be RNA, mRNA, DNA or cDNA.

In some embodiments, the nucleic acids of the invention include expression vectors, including plasmids, or viral vectors, or linear vectors, or vectors that integrate into chromosomal DNA. Expression vectors can contain a nucleic acid sequence that enables the vector to replicate in one or more selected host cells.

In some embodiments, the nucleic acids of the invention include transposons for engineering CARs. In some embodiments, the transposon is the “Sleeping Beauty” transposon system for engineering CAR as described in Trends Gen. (33), 852-870, (2017), herein incorporated by reference. The Sleeping Beauty transposon system only requires one step of plasmid transfection/electroporation, without no virus infection.

In some embodiments, the promoter which is capable of expressing an engineered CAR in a mammalian T cell is the EF1α promoter. The native EF1α promoter drives expression of the alpha subunit of the elongation factor-1 complex, which is responsible for the enzymatic delivery of aminoacyl tRNAs to the ribosome. The EF1α promoter is effective in driving CAR expression from transgenes cloned into a lentiviral vector. (Milone et al., Mol. Ther. 17(8): 1453-1464(2009), herein incorporated by reference). In some embodiments, the promoter is the immediate early cytomegalovirus (CMV) promoter sequence. The CMV promoter sequence is a strong constitutive promoter sequence capable of driving high levels of expression of any polynucleotide sequence operatively linked thereto. In some embodiments, the promoter sequence is selected from the simian virus 40 (SV40) early promoter, mouse mammary tumor virus promoter (MMTV), human immunodeficiency virus (HIV) long terminal repeat (LTR) promoter, MoMuLV promoter, phosphoglycerate kinase (PGK) promoter, MND promoter (a synthetic promoter that contains the U3 region of a modified MoMuLV LTR with myeloproliferative sarcoma virus enhancer, (Li et al., J. Neurosci. Methods vol. 189, pp. 56-64(2010), incorporated by reference), an avian leukemia virus promoter, an Epstein-Barr virus immediate early promoter, a Rous sarcoma virus promoter, human gene promoters (which can include or exclude: the actin promoter, the myosin promoter, the elongation factor-1a promoter, the hemoglobin promoter, and the creatine kinase promoter) and combinations thereof.

In some embodiments, expression vectors have promoter elements, (e.g., enhancers) to regulate the frequency of transcriptional initiation. In some embodiments, the promoter elements are located in the region 30-110 bp upstream of the start site, although a number of promoters have been shown to contain functional elements downstream of the start site as well. The spacing between promoter elements frequently is variable, so that promoter function is preserved when elements are inverted or moved relative to one another. Individual elements of the promoter can function either cooperatively or independently to activate transcription.

In some embodiments, the polynucleotides encoding the engineered CAR include polynucleotide sequences that are substantially equivalent to the polynucleotide sequences described herein. Polynucleotides according to the invention have at least about 80%, 90%, or 95% sequence identity to a polynucleotide sequence described herein. The invention also provides the complement of the polynucleotides including a nucleotide sequence that has at least about 80%, 90%, or 95%, sequence identity to a polynucleotide encoding a polypeptide recited above. The polynucleotide can be DNA (genomic, cDNA, amplified, or synthetic) or RNA.

The polynucleic acids of this disclosure can be linked to another polynucleic acid so as to be expressed under control of a suitable promoter. The polynucleic acid of this disclosure can be also linked to one or a plurality of regulatory elements that cooperate with a promoter or a transcription initiation site to achieve efficient transcription of the nucleic acid. In some embodiments, the one or a plurality of regulatory elements can include or exclude an enhancer sequence, a polyA site, or a terminator sequence. In some embodiments, the one or a plurality of regulatory sequence can include or exclude a P2A sequence for the co-expression of multiple genes in the vector. In some embodiments, the one or a plurality of regulatory sequence can include or exclude the IRES sequence (Internal Ribosome Entry Site). In some embodiments, the one or a plurality of regulatory sequence can include or exclude LTR sequences at the terminus of the vector sequence.

In some embodiments, the polynucleic acid encoding the engineered CAR is inserted into a vector, and the vector is introduced into a cell. In some embodiments, the polynucleic acid encoding the engineered CAR is introduced to a eukaryotic cell by transfection (e.g., Gorman, et al. Proc. Natl. Acad. Sci. 79.22 (1982): 6777-6781, herein incorporated by reference), transduction (e.g., Cepko and Pear (2001) Current Protocols in Molecular Biology unit 9.9; DOI: 10.1002/0471142727.mb0909s36, herein incorporated by reference), calcium phosphate transformation (e.g., Kingston, Chen and Okayama (2001) Current Protocols in Molecular Biology Appendix 1C; DOI: 10.1002/0471142301.nsa01cs01, herein incorporated by reference), cell-penetrating peptides (e.g., Copolovici, Langel, Eriste, and Langel (2014) ACS Nano 2014 8(3), 1972-1994; DOI: 10.1021/nn4057269, herein incorporated by reference), electroporation (e.g Potter (2001) Current Protocols in Molecular Biology unit 10.15; DOI: 10.1002/0471142735.im 1015s03 and Kim et al (2014) Genome 1012-19. doi:10.1101/gr.171322.113, Kim et al. 2014 describe the Amaza Nucleofector, an optimized electroporation system, both of which are herein incorporated by reference), microinjection (e.g., McNeil (2001) Current Protocols in Cell Biology unit 20.1; DOI: 10.1002/0471143030.cb2001s18, herein incorporated by reference), liposome or cell fusion (e.g., Hawley-Nelson and Ciccarone (2001) Current Protocols in Neuroscience Appendix 1F; DOI: 10.1002/0471142301.nsa01fs10, herein incorporated by reference), mechanical manipulation (e.g., Sharon et al. (2013) PNAS 2013 110(6); DOI: 10.1073/pnas.1218705110, herein incorporated by reference). In some embodiments, the engineered CAR polynucleic acid can be transiently expressed episomally, or can be integrated into the genome of the eukaryotic cell using recombination (e.g., Lisby and Rothstein (2015) Cold Spring Harb Perspect Biol. March 2; 7(3). pii: a016535. doi: 10.1101/cshperspect.a016535, herein incorporated by reference), or non-homologous integration (e.g., Deyle and Russell (2009) Curr Opin Mol Ther. 2009 August; 11(4): 442-7, herein incorporated by reference).

In some embodiments, the nucleic acid encoding the engineered CAR is integrated into the eukaryotic cell chromosome at a genomic safe harbor site. The genomic safe harbor site can include or exclude CCR5, AAVS1, human ROSA26, or PSIP1 loci. (Sadelain et al., Nature Rev. 12:51-58 (2012); Fadel et al., J. Virol. 88(17): 9704-9717(2014); Ye et al., PNAS 111(26): 9591-9596(2014), all of which are herein incorporated by reference). In some embodiments, the integration of the polynucleic acid encoding the engineered CAR is done using a gene editing system. The gene editing system can include or exclude CRISPR, TALEN, or Zinc-Finger nuclease systems.

In some embodiments, inserting the gene encoding the engineered CAR is done via transduction. Transduction can be done with a virus vector selected from a retrovirus vector (including an oncoretrovirus vector, a lentivirus vector, or a pseudo type vector), an adenovirus vector, an adeno-associated virus (AAV) vector, a simian virus vector, a vaccinia virus vector or a sendai virus vector, an Epstein-Barr virus (EBV) vector, or adeno-associated virus and a HSV vector. In some preferred embodiments, the virus vector lacks the replicating ability so as not to self-replicate in a transfected cell. The vector comprising the gene encoding the engineered CAR can also be referred to as a “recombinant virus.”

In some embodiments, when a retrovirus vector is used, the process of this disclosure can be carried out by selecting a suitable packaging cell based on a LTR sequence and a packaging signal sequence possessed by the vector and preparing a retrovirus particle using the packaging cell. The packaging cell can include or exclude: PG13 (ATCC CRL-10686), PA317 (ATCC CRL-9078), GP+E-86 and GP+envAm-12 (U.S. Pat. No. 5,278,056, herein incorporated by reference), and Psi-Crip (Proceedings of the National Academy of Sciences of the United States of America, vol. 85, pp. 6460-6464(1988), herein incorporated by reference). In some embodiments, the retrovirus particle can be prepared using a 293 cell or a T cell having high transfection efficiency.

Viral based systems are used for gene transfer into mammalian cells. A selected gene can be inserted into a vector and packaged in viral particles. In some embodiments, the recombinant virus can then be isolated and delivered to cells of the subject either in vivo or ex vivo. In some embodiments, lentivirus vectors are used to deliver genes into T cells. In some embodiments, adenovirus vectors are used to deliver genes into T cells.

The modified oligonucleotides (RNA and/or DNA) described herein can be synthesized and/or modified by methods well established in the art, including those described in “Current Protocols in Nucleic Acid Chemistry,” Beaucage, S. L. et al. (Edrs.), John Wiley & Sons, Inc., New York, N.Y., USA, which is hereby incorporated herein by reference in its entirety. Transcription methods are described further herein in the Examples. In one embodiment, a template for an ssDNA is synthesized using “splint-mediated ligation,” which allows for the rapid synthesis of DNA constructs by controlled concatenation of long oligos and/or dsDNA PCR products and without the need to introduce restriction sites at the joining regions. In some embodiments, the foregoing process adds generic untranslated regions (UTRs) to the coding sequences of genes during T7 template generation. In some embodiments, splint mediated ligation adds nuclear localization sequences to an open reading frame, makes dominant-negative constructs with point mutations starting from a wild-type open reading frame. Briefly, single-stranded DNA (“ssDNA”) and/or denatured dsDNA components are annealed to splint oligos which bring the desired ends into conjunction, the ends are ligated by a thermostable DNA ligase and the desired constructs amplified by PCR. A synthetic, modified oligonucleotide is then synthesized from the template using an RNA polymerase in vitro. After synthesis of a synthetic, modified oligonucleotide is complete, the DNA template is removed from the transcription reaction prior to use with the methods described herein. In some embodiments of these aspects, the synthetic oligonucleotides are further treated with an alkaline phosphatase.

In some embodiments, as an alternative approach to cysteine modification or sortase-mediated ligation, the non-canonical amino acid 4-azidophenylalanine is introduced into the proteins via amber codon suppression during the cell culturing to enable modification with cyclooctyne-DNA. In some embodiments, the length of the staple strand sequence is modulated to select for accessible ECM-degrading proteins immobilized to the DNA nanostructure.

### Large Scale Economic Production of DNA Nanostructures

This disclosure provides for methods of large scale production of DNA nanostructures. One of the most challenging obstacles to transforming DNA nanostructures from mere curiosities into real-world solutions was the cost of synthetic DNA strands and the small production scales of DNA origami structures. In some embodiments, a phage-based biotechnology method enables the economic manufacture of the short DNA staple strands and DNA nanostructures. Bacteriophages are employed to produce single-stranded precursor DNA that contains a plurality of target strand sequences. In some embodiments, the DNA strands and oligonucleotides are prepared by the methods described in Praetorius F, Kick B, Behler K L, Honemann M N, Weuster-Botz D, Dietz H. Biotechnological mass production of DNA origami. Nature. 2017; 552(7683): 84-+. doi: 10.1038/nature24650. PubMed PMID: WOS: 000417560500047, herein incorporated by reference.

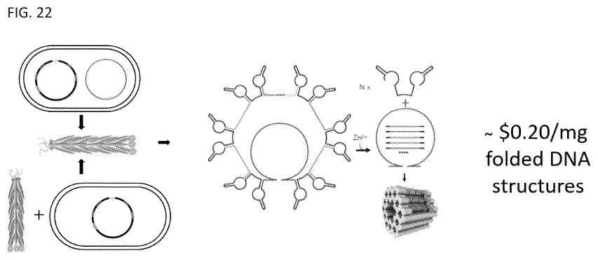

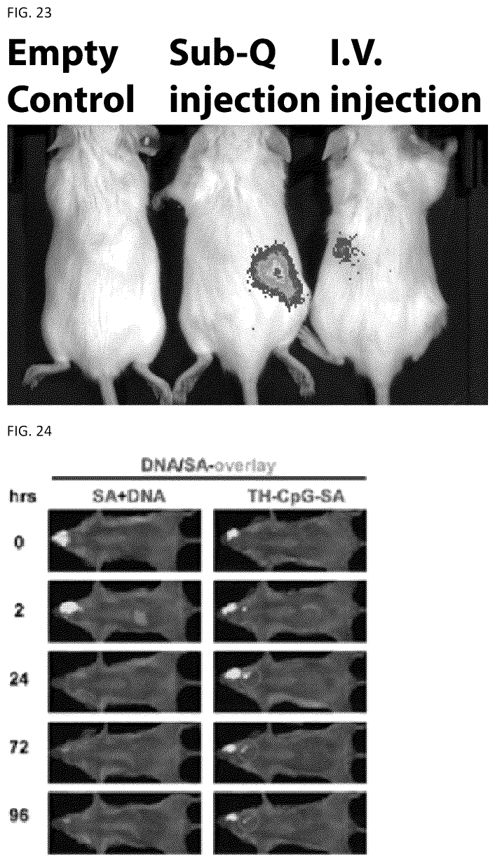

A piece of self-excising DNAzyme sequence was inserted between each two adjacent target strands, allowing the cleavage products to self-assemble into prescribed DNA origami structures, as shown in FIG. 22. Compared with traditional solid-phase DNA synthesis methods, the phage-based strategy reduced the costs of folded DNA origami structures from ˜$200/mg to ˜$0.2/mg to enable economic DNA nanotechnology production methods.

### Eukaryotic Cells Expressing the Engineered CAR

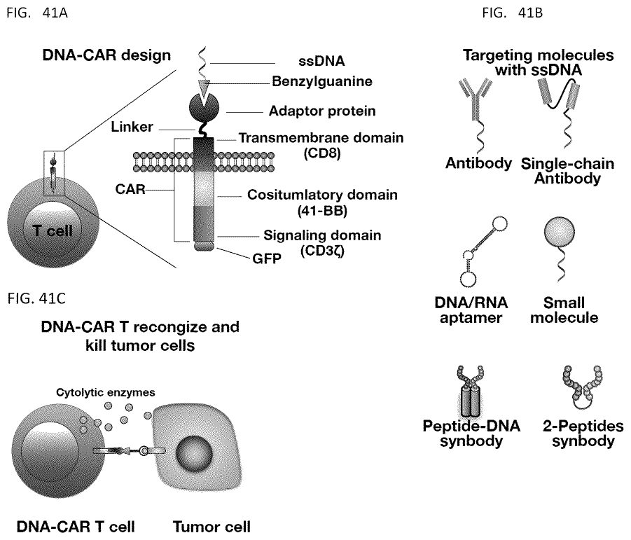

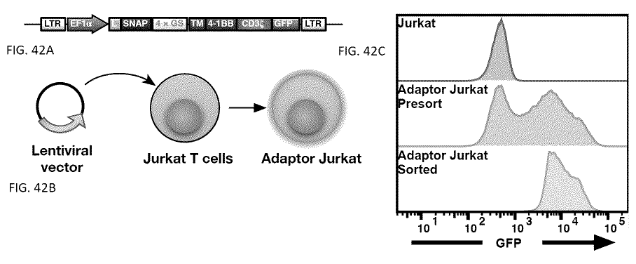

The cell expressing the engineered CAR of this disclosure is a cell in which a nucleic acid encoding an engineered CAR is introduced and expressed. In one embodiment, the cell is an engineered T cell as shown in FIG. 41. In some embodiments, the engineered CAR comprises a first ssDNA oligonucleotide strand as a first oligonucleotide which can hybridize with a second oligonucleotide which is conjugated to a targeting agent and/or targeting molecule. The first ssDNA oligonucleotide is chemically conjugated to a protein tag. In some embodiments, the protein tag is a benzylguanine. The benzylguanine can react with an adaptor protein which is expressed as part of the engineered CAR on the extracellular domain. The adaptor protein can be selected from SNAP-Tag™, CLIP-Tag™, or HALO-Tag™. In some embodiments, the adaptor protein is linked via a linker to a CD8 transmembrane domain which is linked to a 41-BB costimulatory domain and a CD3-zeta signaling domain. Optionally, the engineered CAR further comprises a GFP, and optionally further comprises an antibiotic resistant gene. FIG. 41A shows one embodiment of the engineered T cell comprising an engineered CAR (“DNA CAR”). FIG. 41B shows optional embodiments where targeting molecules are conjugated to engineered T cells. Targeting molecules can include or exclude: antibodies, single chain antibodies, DNA or RNA aptamers, small molecules, peptide synthetic antibodies (synbody) which target receptors on cell surface. In some embodiments, the targeting molecules are conjugated with a BG-ssDNA strand, e.g., poly A, which hybridizes to DNA-CAR T cells. FIG. 41C depicts the mechanism by which engineered T cells recognize tumor cells and activate. Engineered T cells comprising aptamers as the targeting molecule can recognize tumor antigens on their surface, the engineered T cell is activated by tumor cells and releases cytokines (e.g., interferon γ, IFN-γ), and cytolytic enzymes (e.g., granzyme B).

In some embodiments, the engineered T cell expresses two or more adaptor proteins. The two or more adaptor proteins may, in some embodiments, be co-expressed using, for example, the CAR constructs depicted in FIG. 18. Each of the adaptor proteins can be the same or different. In some embodiments, the respective protein tag for each co-expressed adaptor protein can comprise the same or a different “first” ssDNA oligonucleotide to which targeting agents comprising second oligonucleotides of which a portion are complementary to a portion of the same or different “first” ssDNA oligonucleotide sequences.

In some embodiments, a eukaryotic cell of this disclosure binds to a specific antigen via the targeting compound to transmit a signal into the eukaryotic cell, and as a result, the eukaryotic cell is activated. The activation of the eukaryotic cell expressing the engineered CAR is varied depending on the kind of a eukaryotic cell and the intracellular element of the engineered CAR, and can be confirmed based on, for example, release of a cytokine, release of a reporter molecule (e.g., luciferin or luciferase with substrate), improvement of a cell proliferation rate, change in a cell surface molecule, or the like as an index. In some embodiments, release of a cytotoxic cytokine (a tumor necrosis factor, lymphotoxin, etc.) from the activated cell causes destruction of a target cell expressing an antigen. In addition, release of a cytokine or change in a cell surface molecule stimulates other immune cells, for example, a B cell, a dendritic cell, a NK cell, and/or a macrophage.

In some embodiments, eukaryotic cells expressing the engineered CAR connected to a targeting molecule or to a targeting agent comprising a targeting molecule are detected using Jurkat-Lucia™ NFAT cells (Invivogen) which encode a luciferase gene upon activation. Optical detection of luciferase activity indicates activation of the engineered T cell expressing the engineered CAR and comprising the targeting agent when the targeting agent is bound to the target ligand.

In some embodiments, the targeting molecule is associated with the engineered T cell by a means other than oligonucleotide immobilization. The adaptor tag, in some embodiments, is conjugated with a carbohydrate and the targeting agent is conjugated to a leptin. In some embodiments, the adaptor tag is conjugated with a leptin and the targeting agent is conjugated to a carbohydrate. In some embodiments, the adaptor tag/targeting agent interaction is a fluoro-fluoro interaction, where the adaptor tag is conjugated to a first perfluoroalkane molecule and the adaptor tag is conjugated to a second perfluoroalkane molecule. In some embodiments, the adaptor tag is conjugated to a lipid and the targeting agent is conjugated to a cognate lipid recognition binding agent. In some embodiments, the lipid recognition binding agent is a second lipid.

### DNA Nanostructures

In some embodiments, this disclosure includes a targeting agent that comprises a DNA nanostructure and a targeting molecule. The DNA nanostructure relies on DNA self-assembly. There are several reasons for the success of DNA in nano-construction. First, Watson-Crick base-pairing between complementary DNA strands is highly predictable and stable. Second, the geometric features of the DNA double-helix is appropriate for nanoscale assembly: the helix is roughly 2 nm in diameter and 3.4 nm pitch per helical repeat. In addition, the development of several user-friendly software interfaces has facilitated the modeling of even the most intricate DNA nanostructures for experimental testing. Third, modern organic chemistry and molecular biology provide a diverse toolbox to readily synthesize, modify, and replicate DNA molecules at a relatively low cost. The biocompatibility of DNA makes it suitable for the construction of multicomponent nanostructures made from hetero-biomaterials with designed functions.

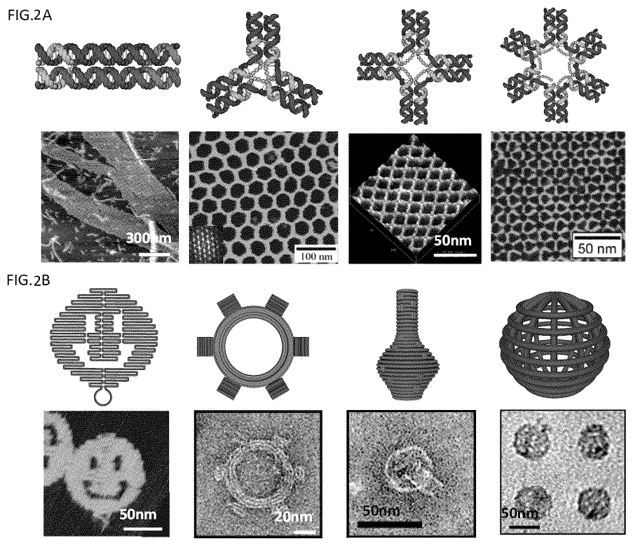

In some embodiments, the DNA nanostructure for use with any of the embodiments described herein is based on the principle of DNA tile assembly (Winfree E, Liu F R, Wenzler L A, Seeman N C. Design and self-assembly of two-dimensional DNA crystals. Nature. 1998; 394(6693): 539-44. doi: Doi 10.1038/28998. PubMed PMID: ISI: 000075238700036; He Y, Chen Y, Liu H P, Ribbe A E, Mao C D. Self-assembly of hexagonal DNA two-dimensional (2D) arrays. Journal of the American Chemical Society. 2005; 127(35): 12202-3. doi: Doi 10.1021/Ja0541938. PubMed PMID: ISI: 000231637100027; Yan H, Park S H, Finkelstein G, Reif J H, LaBean T H. DNA-templated self-assembly of protein arrays and highly conductive nanowires. Science. 2003; 301(5641): 1882-4. doi: DOI 10.1126/science. 1089389. PubMed PMID: ISI: 000185536700043; He Y, Tian Y, Ribbe A E, Mao C D. Highly connected two-dimensional crystals of DNA six-point-stars. Journal of the American Chemical Society. 2006; 128(50): 15978-9. doi: Doi 10.1021/Ja0665141. PubMed PMID: ISI: 000242825600025, each of which is incorporated by reference). DNA tile assembly is a versatile and powerful method wherein small building blocks of DNA tiles, motifs, or even single-stranded DNA—along with the interactions between the building blocks—can be pre-designed using sticky-end cohesion. The recognition between sticky ends are programed with several algorithms that guide DNA building blocks to self-assemble into large 2D arrays with diverse topological and geometric features (FIG. 2A). In some embodiments, the DNA nanostructure is based on the principle of scaffolded DNA-origami (Rothemund PWK. Folding DNA to create nanoscale shapes and patterns. Nature. 2006; 440(7082): 297-302. doi: Doi 10.1038/Nature04586. PubMed PMID: WOS: 000235997600044; Douglas S M, Dietz H, Liedl T, Hogberg B, Graf F, Shih W M. Self-assembly of DNA into nanoscale three-dimensional shapes. Nature. 2009; 459(7245): 414-8. doi: Doi 10.1038/Nature08016. PubMed PMID: ISI: 000266243700043; Han D R, Pal S, Nangreave J, Deng Z T, Liu Y, Yan H. DNA Origami with Complex Curvatures in Three-Dimensional Space. Science. 2011; 332(6027): 342-6. doi: DOI 10.1126/science. 1202998. PubMed PMID: ISI: 000289516600042; Han D R, Pal S, Yang Y, Jiang S X, Nangreave J, Liu Y, Yan H. DNA Gridiron Nanostructures Based on Four-Arm Junctions. Science. 2013; 339(6126): 1412-5. doi: DOI 10.1126/science. 1232252. PubMed PMID: ISI: 000316740700037, each of which is incorporated by reference). Scaffolded DNA origami is a technique which enables the folding of a long, single-stranded M13mp18 genomic DNA into a designed target shape by using a plurality of short DNA strand sequences referred to herein as “staple strands” to form a DNA origami nanostructure. In some embodiments, the folding process is programmed resulting in the formation of structures in near-quantitative yields for many designs with unpurified staple strands. The inventors have surprisingly discovered that DNA origami objects are fully addressable due to the unique sequence of long genomic DNA, where each staple strand results in possible modification positions that can be linked to other functional materials with useful biological or electronic properties. In some embodiments, a first staple strand comprises a polynucleotide with a portion of which is complementary to the first oligonucleotide, wherein the first oligonucleotide is connected to the engineered T cell. In some embodiments, the DNA origami further comprises a second staple strand which is a third oligonucleotide which comprises a portion which is complementary to a fourth oligonucleotide where the fourth oligonucleotide is conjugated to a targeting molecule. In some embodiments, the targeting molecule is selected from an aptamer, protein, cytokine, chemokine, lipid, oligonucleotide (which can include or exclude: ssDNA, dsDNA, RNA, nucleic acid-hybrids), protein, synbody, antibody or fragment thereof, or combinations thereof. In some embodiments, the long genomic strand can be RNA. In some embodiments, when the long genomic strand is DNA. In certain embodiments, the DNA origami nanostructure comprising a central polynucleotide strand and a first staple strand which comprises the second oligonucleotide sequence and a plurality of second staple strands which comprises one or a plurality of third distinct oligonucleotide sequences; and one or more targeting molecules connected to one or a plurality of fourth distinct oligonucleotide sequence(s). In certain embodiments, a portion of a third distinct oligonucleotide sequence is complementary to a portion of a distinct fourth oligonucleotide sequence. Therefore, a targeting molecule conjugated to a fourth distinct oligonucleotide sequence can be positioned at the location of the complementary third distinct oligonucleotide sequence. In some embodiments, there are a plurality of distinct targeting molecules each of which is conjugated to one of a plurality of distinct fourth oligonucleotides which comprises a portion, each of which is complementary to a portion of one of a plurality of third distinct oligonucleotides.

In certain embodiments, this disclosure includes a DNA-based nanoscale scaffold to augment T cells with additional functions for targeting solid tumors. These functions can include or exclude: 1) rapid assembly of one or more non-genetically encodable targeting molecules; 2) incorporation of multiple types of molecules, including targeting ligands and enzymes for matrix degradation; 3) precise control over stoichiometry and nanoscale presentation of said targeting molecules; and 4) dramatically reduced time for production of engineered T cells by bypassing the requirement for genetic engineering of novel CAR constructs.

This disclosure provides for methods of using DNA nanostructures as platforms to organize modular functional molecules to demonstrate the enablement of using engineered T cells to kill cancer cells and to activate engineered T cells in the presence of a cancer cell.

In some embodiments, the DNA nanostructure has a shape selected from a semi-flat or flat sheet which may be in the form of a square or rectangle, a tube, a sphere, a pyramid, a box, a rectangle, and a round flat disc. In some embodiments, the DNA origami nanostructures are selected from the structures shown in FIG. 2, 5, 13, 14, or 16. The DNA nanostructure can be designed using algorithms to generate appropriate sequences for customized DNA structures to minimize unwanted interactions between DNA strands and help achieve a high folding yield of designed structures. Design parameters including the size, shape, and helical directions of DNA nanostructures can be optimized to improve targeting molecule loading efficiency of DNA nanostructures. The assembly of such DNA structures is achieved through thermal annealing processes and the obtained structures are purified with dialysis spin columns, gel electrophoresis methods, or size-exclusion chromatography methods. Atomic force microscopy (AFM) confirms the correct formation of the designed DNA nanostructure. In some embodiments, the DNA or RNA nanostructure can include or exclude those in U.S. Pat. Nos. 8,440,811; 8,552,167; PCT App. No. PCT/US18/48973, PCT/US18/48976, U.S. application Ser. No. 16/208,103, and PCT App. No. PCT/US19/13118, all of which are herein incorporated by reference.

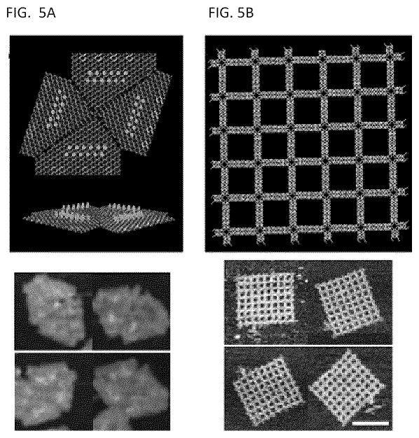

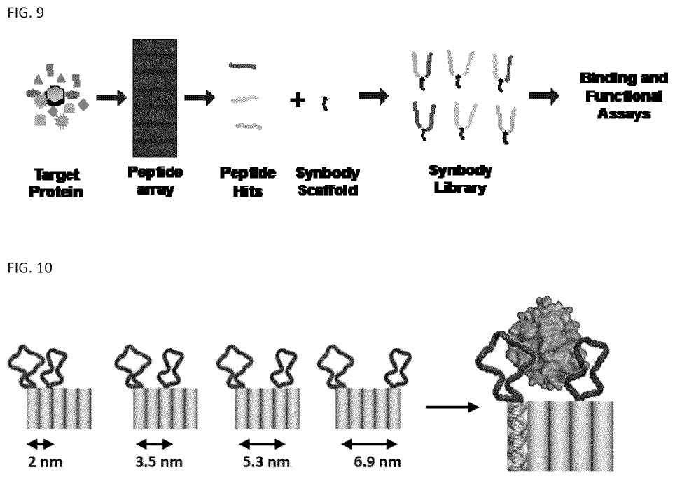

In some embodiments, the DNA nanostructures comprise a plurality of unique staple strands where each unique staple strand can serve as an addressable surface location to organize modular functional targeting molecules, including MMP proteins, T cell recruiting chemokines, or T cell proliferation cytokines. As shown in FIG. 5A, different targeting molecules are attached to the DNA nanostructure with a specific DNA sequence (“second oligonucleotide”) that is comprises a portion which is complementary to a portion of the sequence of staple strand sequence protruding from a DNA nanostructure (“first oligonucleotide”), whereby all the geometric parameters are precisely designed and have an addressable surface. In some embodiments, the staple strand sequences are oriented based on the conformations of the DNA nanostructure. The resulting construct enables the configuration of targeting molecules, including the distance between said molecules, their multivalency, and their relative geometry. As shown in FIG. 10, rows of different staple strands can be created to be at a precise distance apart to achieve optimal binding distance to increase the avidity of the targeting molecules to their cognate antigen.

In some embodiments, the DNA nanostructures are comprised of phosphorothioate backbones or locked nucleic acids to prevent DNA degradation by nucleases. In other embodiments, the DNA nanostructures can comprise an electrostatic coating (e.g., oligolysine-PEG conjugate), to simultaneously stabilize DNA nanostructures to low-magnesium conditions (including those encountered in the bloodstream) and help reduce degradation by endonucleases.

### Design and Optimization of Adaptor Proteins for Engineered T Cells

In some embodiments, the viral vector encoding the engineered CAR comprises a plurality of orthogonal adaptor protein sequences to conjugate addition DNA strand on engineered T cell surfaces. In some embodiments, the orthogonal adaptor protein is CLIP-Tag™, which is orthogonal to SNAP-Tag™, or HALO-Tag™ which is orthogonal to both CLIP-Tag™ and SNAP-Tag™. CLIP-Tag™ is engineered from the SNAP-Tag™ protein to accept 02-benzylcytosine derivatives as substrates, instead of O6-benzylguanine (for SNAP-Tag™).

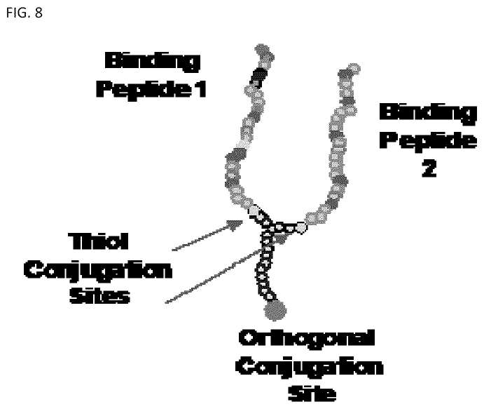

In some embodiments, the viral vector encoding the engineered CAR comprises a SNAP-Tag™ tag for DNA conjugation connected to a CAR structure comprising a CD8 transmembrane domain, a 41BB co-stimulatory domain, a CD34 signaling domain, and a CLIP-Tag™ adaptor protein as a second adaptor protein for different DNA strand conjugation, and optionally a luciferase gene for reporting the engineered CAR incorporation, as shown in FIG. 7. The orthogonal adaptor protein (e.g., CLIP-Tag™) expresses on the surface of T cells, exposing a SNAP-Tag™ adaptor protein that is used to conjugate DNA modified with an O6-benzylguanine moiety and CLIP-Tag™ adaptor protein that is used to conjugate DNA modified with an 02-benzylcytosine moiety.

### Design and Optimization of Engineered CAR for Engineered T Cell Immunotherapy

In some embodiments, the adaptor protein is the SNAP-Tag™ protein, a protein derived from the 20 kDa DNA repair protein O⁶-alkylguanine-DNA alkyltransferase (AGT), and which can be labeled using O⁶-benzylguanine derivatives. SNAP-Tag™ tags are covalently labeled with O⁶-benzylguanine (BG) derivatives comprising a chemical probe by undergoing an irreversible reaction in which the functionalized benzyl group of the BG derivative is transferred to an active site cysteine to form a covalently modified protein. Thus, by using BG-connected DNA strands as a first oligonucleotide presents a construct which can be easily functionalized by hybridizing a second oligonucleotide connected to a targeting molecule where a portion of the second oligonucleotide sequence is complementary to a portion of the first oligonucleotide sequence. The second oligonucleotide is connected to the targeting molecule using a cross-functional linker (e.g., sulfo-SMCC), which links a thio-modified oligonucleotide to an amine (e.g., lysine) on the targeting molecule. In some embodiments, the engineered CAR comprises a SNAP-Tag™ protein which is expressed on the T cell surface to attach DNA nanostructures comprising multiple ligands.

In some embodiments, the cross-functional linker is selected from: SIA, SBAP, SIAB, Sulfo-SIAB, AMAS, BMPS, GMBS, Sulfo-GMBS, MBS, Sulfo-MBS, SMCC, Sulfo-SMCC, EMCS, Sulfo-EMCS, SMPB, Sulfo-SMPB, SMPH, LC-SMCC, Sulfo-KMUS, SPDP, LC-SPDP, Sulfo-LC-SPDP, SMPT, PEG4-SPDP, PEG12-SPDP, DCC, EDC, NHS, Sulfo-NHS, BMPH, EMCH, MPBH, KMUH, PDPH, ANB-NOS, Sulfo-SANPAH, SDA, Sulfo-SDA, LC-SDA, Sulfo-LC-SDA, SDAD, Sulfo-SDAD, NHS-Azide, NHS-PEG4-Azide, and NHS-Phosphine (all of which are commercially available from ThermoFisher). In some embodiments, the cross-functional linker can be selected from Alkyne-PEG5-acid, Allyl (4-methoxyphenyl)dimethylsilane, 4-Aminobutyraldehyde diethyl acetal, (E)-N-(2-Aminoethyl)-4-{2-[4-(3-azidopropoxy)phenyl]diazenyl}benzamide hydrochloride, N-(2-Aminoethyl) maleimide trifluoroacetate salt, Amino-PEG4-alkyne, Benzyl N-(3-hydroxypropyl) carbamate, 4-(Boc-amino) butyl bromide, 2-[2-(Boc-amino) ethoxy]ethoxyacetic acid (dicyclohexylammonium) salt, 2-(Boc-amino) ethyl bromide, 6-(Boc-amino) hexyl bromide, 3-(Boc-amino) propyl bromide, N-(4-Bromobutyl) phthalimide, 4-Bromobutyryl chloride, N-(2-Bromoethyl) phthalimide, 3-(Bromomethyl)benzoic acid N-succinimidylester, 4-(Bromomethyl) phenyl isothiocyanate, N-(3-Bromopropyl) phthalimide, tert-Butyl 2-(4-{[4-(3-azidopropoxy)phenyl]azo}benzamido) ethylcarbamate, 2-[2-(tert-Butyldimethylsilyloxy) ethoxy]ethanamine, tert-Butyl 4-hydroxybutyrate, N-(2-Hydroxyethyl) maleimide, and 2-Maleimidoethyl mesylate (all of which are commercially available from Sigma Aldrich).

In some embodiments, the adaptor protein (e.g., SNAP-Tag™ protein) is used to chemically conjugate an oligonucleotide (e.g., ssDNA) to an engineered CAR structure comprising a CD8 transmembrane domain, a 41BB co-stimulatory domain, a CD34 signaling domain and a luciferase gene, incorporated into a viral vector. In some embodiments, the CAR can have the sequences shown in FIGS. 7 and 18. In some embodiments, the co-stimulatory domain can include or exclude CD28, OX40, and 4-1BB. The adaptor protein is expressed on the surface of the engineered T cells, to expose the adaptor protein to be used to connect ssDNA modified with an O⁶-benzylguanine moiety. In some embodiments, the engineered CAR can include the puromycin resistance gene to allow for simple selection of effectively transduced cells by addition of puromycin. In some embodiments, the viral vector is a lentiviral vector. A control lentiviral vector encodes only a SNAP-Tag™ protein and a CD8 transmembrane domain. In some embodiments, the lentivirus vector also includes a luciferase gene (“Luc”) to allow for facile T cell tracking in vivo. In some embodiments, the lentivirus vector can include a leader sequence “L” between the promoter sequence and the protein tag sequence to provide for separation between the two functional domains.

### Screening Targeting Ligands for Targeting Solid Tumor Markers-Synbodies and Aptamers

One of the key obstacles for CAR T cell therapy to treating solid tumors is the identification of suitable neo-antigens or TAAs (“tumor-associated antigens”) to serve as targets. The biological heterogeneity of solid tumor malignancies does not lend itself to a “one antigen fits all” approach. This difficulty is compounded by the frequent expression of putative target antigens on normal tissues that leads to on-target, off-tumor toxicity. Despite these limitations, acceptable antigens including CD133, EGFR variant III (EGFRIII), GD2, mucin 1 (MUC-1), mucin 16 (MUC-16), carcinoembryonic antigen, mesothelin (MSLN), and prostate-specific membrane antigen (PSMA) have been characterized and are in various stages of clinical development. However, aside from identification of a suitable TAA, trafficking administered T cells to the tumor is another key challenge to effective therapy.

In some embodiments, the targeting molecule is selected from monoclonal antibodies (mAb), single-chain variable fragments (scFv), and Synbodies. Synbodies are peptide affinity ligands. Synbodies are small (˜6-8 kDa), bivalent peptides that bind their target with antibody-like affinity. Once the composition of a synbody is discovered, large (gram-scale) production is feasible. The small molecular weight of the synbody means that 10 mg of synbody contains the same molar equivalent of targeting molecules as ˜200 mg of mAb (which has a molecular weight of about 200 kDa to about 600 kDa). In some embodiments, the selected synbodies used as targeting molecules are obtained from a screening assay. Synbody screening proceeds in a process by which the target protein is screened against an arrayed library of peptides (125,000 17aa peptides on 0.5 cm² chip) to identify low affinity binders for the target, as shown in FIG. 9). Pairs of low affinity binding peptides are then conjugated to a bivalent synbody scaffold to produce a library of synbodies that are screened for target binding. This system is based on the observation of near cooperative binding that is possible when ligands that bind separate sites on the target protein are linked in the proper orientation. Synbodies are designed with a C-terminal functional group that enables easy modification with affinity tags, fluorescent dyes, or peptides. The separation of the bivalent binding portion of the synbody from the C-terminal conjugation site enables the modification of the synbody while maintaining binding affinity for the target. The screening assay is capable of running multiple development programs in parallel.

DNA nanostructures offer a fully addressable 3D platform to accommodate controlled spatial arrangements of target molecules including protein-bound functional nucleic acids on different regions of the DNA nanostructure.

In some embodiments, the targeting molecule is an aptamer. Aptamers are single-stranded oligonucleotide molecules that mimic antibodies. They are generated from a DNA or RNA library, through an iterative in vitro selection and amplification process known as systemic evolution of ligands by exponential enrichment (SELEX). Unlike conventional nucleic acids, which carry genetic information, aptamers possess intricate tertiary structures and their intramolecular folding enables them to recognize and bind their targets with high affinity and specificity. Aptamers possess several essential advantages in their suitability for clinical application and industrialization. First, aptamers are more economical to produce in large quantities than the equivalent antibodies. Aptamers are made through chemical synthesis, a well-established automated solid-phase method with little to no variation, while antibody synthesis involves different colonies with a high amount of batch-to-batch variation. Second, aptamers are much smaller than protein-based antibodies, allowing them to diffuse through layers of cells in tissues and bind to smaller targets that antibodies cannot reach. Third, aptamers, unlike antibodies, can recover from extreme pH levels and temperatures, because aptamers are self-folded single-stranded DNA (ssDNA) or single-stranded RNA (ssRNA) that lack hydrophobic proteins domains. Fourth, antibodies are typically recognized by the immune system as foreign and often stimulates an undesired immune response, whereas aptamers possess much lower immunogenicity than antibodies and are less likely to provoke negative immune responses. Finally, chemical modifications are easily introduced to aptamers to customize tailored properties in a deterministic way.

In some embodiments, aptamers are incorporated into DNA nanostructures by the methods described herein. Aptamers can comprise a DNA or RNA strand comprising a sequence with a portion of which is complementary to one or a plurality of strand sequences on the DNA nanostructure, or is complementary to the first oligonucleotide connected to the adaptor protein of the engineered CAR. When adaptors are hybridized to DNA nanostructures, they are spatially positioned on the DNA nanostructure such that the distance and angle is identified based on the addressability of the scaffold DNA nanostructures. The inventors have previously discovered that multivalent aptamers can by connected using two aptamers against thrombin into a DNA nanostructure with various precise distances ranging from 2 to 7 nm. It was found that the optimized binding affinity was obtained at an inter-aptamer distance of 5.3 nm, with the final assembles showing a 50-fold improvement in affinity over the monovalent molecules, as shown in FIG. 10.

In some embodiments, the aptamer is an aptamer listed in Table 1.

TABLE 1
Selected aptamers of this disclosure.

| Aptamer | | SEQ |
|---|---|---|
| Name | Sequence (5′→3′) | ID NO. |
| hPD-L1- | ACG GGC CAC ATC AAC TCA TTG ATA | SEQ ID |
| apt1 | GAC AAT GCG TCC ACT GCC CGT | NO: 5 |
| hMUC1- | GCA GTT GAT CCT TTG GAT ACC CTG G | SEQ ID |
| apt1 | | NO: 6 |
| hMUC1- | GCAGT TGATC CTTTG GATAC CCTGG TTT | SEQ ID |
| apt2 | TTA AAA | NO: 7 |
| hMUC1- | GGGAGACAAGAATAAAC GCT CAA GCA GTT | SEQ ID |
| apt3 | GAT CCT TTG GAT ACCCTGGTTC GAC | NO: 8 |
| | AGG AGG CTC ACA ACA GGC | |
| hEGFR- | TACCAGTGCGATGCTCAGTGCCGTTTCT TCT | SEQ ID |
| apt1 | CTT TCG CTT TTT TTGCTTTTGAGC | NO: 9 |
| | ATGCTGACGCATTCG GTT GAC | |
| Scg8 | ATC TAA CTG CTG CGC CGC CGG GAA | SEQ ID |
| | AAT ACT GTA CGG TTA GA | NO: 10 |
| Control | AAAAAAAAAAAAAAACGTGCAGTACGCCA | SEQ ID |
| Aptamer | ACC TTT CTC ATG CGCTGCCCCTCTTA | NO: 11 |

The synbody development consists of 3 major steps: (i) peptide selection and synthesis, (ii) synbody library construction, (iii) screening and validation for synbody selection.

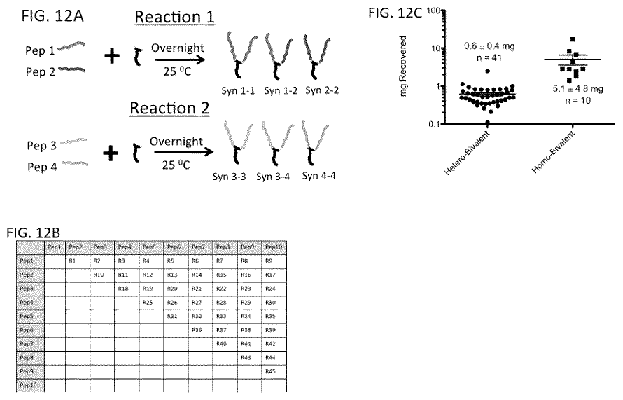

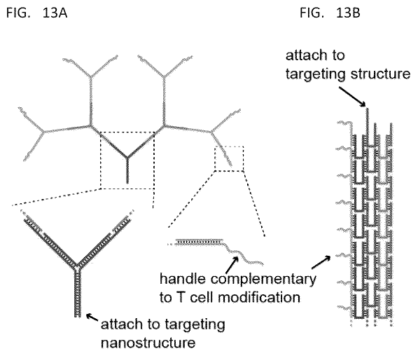

The synbody development process begins with purchasing the target protein (˜250 μg) from commercial vendors. Upon receipt, the protein is aliquoted and stored according to specifications. The target protein is then labeled with AlexaFluor 555 and screened against an in situ synthesized peptide microarray composed of 125,000 unique 17-mer peptides of random amino acid sequence that contain both D- and L-amino acids. Peptide arrays are synthesized in the Center for Innovations in Medicine. The array format is such that the 125,000 peptide library is synthesized 24 times on a slide of standard dimensions in individually addressable arrays, as shown in FIG. 12A. This enables replicate assays to be included as well as more complex assays, as illustrated in FIG. 12B, in which TNFα was fluorescently labeled and screened on replicate arrays (x-axis). In the same assay, TNFα was complexed with TNF-receptor 1 (TNFR1) and screened on replicate arrays (y-axis). In this way, peptides that bind to TNFα in the TNFR 1 binding site is blocked and their signals are lower in the TNFα/TNFR1 condition (FIG. 12C). While this assay format enables identification of peptides for use as synbody inhibitors, identification of receptor inhibiting peptides is not necessary for the proteins in Table 2, which can used for tumor targeting. Each peptide array has a number of control peptides included that provide measures of array-to-array reproducibility that are independent of the target protein. In some embodiments, the protein target is screened on peptide arrays to identify the proper peptide pairs.

In some embodiments, synbodies are constructed through the use of a conjugation rather than synthetic approach. Thiol conjugation provides a simple method to produce libraries of synbodies in a short period of time. Each binding peptide is synthesized with a terminal cysteine amino acid that is conjugated to a bi-functional peptide scaffold through a linker (e.g., sulfo-SMCC, or any of the cross-functional linkers described herein), as shown in FIG. 12A. Addition of two binding peptides produces three synbodies in a single reaction in a 1:2:1 ratio (A-A:A-B:B-B).

For example, the pairwise combination of 10 peptides (as shown in FIG. 12B) produces all 55 possible synbodies from 45 reactions. For 15 peptides, a synbody library of X hetero-bivalent synbodies and Y homo-bivalent synbodies are formed. The products of each synbody reaction are purified by HPLC or solution-based extraction methods, analyzed by mass spectrometry to confirm molecular weight, and lyophilized prior to use.

In some embodiments, to identify synbodies that bind the target protein, an SPR screen is used against the immobilized target proteins. In one embodiment, the SPR system is the Biacore T200 SPR system.

In some embodiments, the targeting molecules are aptamers targeting the solid tumor biomarkers listed in Table 3. For example, CD133 is a cancer stem cell biomarker that is overexpressed in most solid tumors. Epidermal growth factor receptor (EGFR) is also an antigen over-expressed on the surface of breast, colon, lung and a range of other epithelial cancer cells and an important cancer biomarker. Both of these biomarkers are ideal antigens for clinical applications in cancer diagnosis, prognosis, imaging, and therapy. In some embodiments, high affinity bivalent aptamers against CD133 and EGFR with ssDNA library is identified through SELEX processes. The extracellular CD133 and IgV-like N domain of CD133 has a comparable size to that of thrombin. In some embodiments, the production process of recombinant proteins involves immobilized on magnetic beads either through covalent binding or His-tag trapping. In some embodiments, increasing selection pressure is applied to the selection process to enrich high affinity aptamers. The sequencing result is analyzed to identify high affinity aptamers from their percentage in the sequencing pool.

In some embodiments, the synbody screening process can include first filtering peptides based upon signal intensity for the target protein as well as intensity for other targets. This enables selection of peptides with the highest target selectivity that translates into production of high-affinity, high-selectivity synbodies. The synbody conjugation chemistry is straightforward but reaction yields are sometimes low if there is a problem with the solubility of one or more peptides. In some embodiments, small amount of organic polar aprotic solvent, including acetonitrile, is added to the reaction mixture to increase reaction yield. The synbody can be used with a variety of immobilization methods including the use of amine and carboxy conjugation to the SPR chip to immobilize the protein through different amino acids. In one embodiment, when the target protein has been expressed with an affinity tag, the use of an anti-tag antibody, including anti-His6 if the protein is His tagged is used. By changing the orientation of the protein on the surface, the synbody library can be screened against the full surface of the target protein.

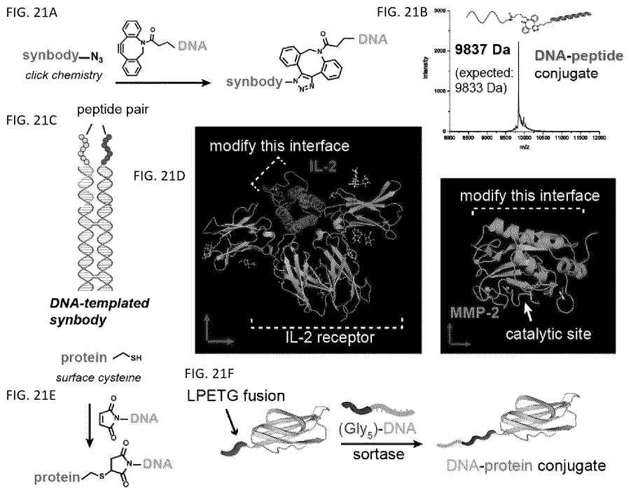

In some embodiments, synbodies are synthesized with a single azide handle in the molecular structure to allow for conjugation to alkynyl-modified DNA via strain-promoted azide-alkyne cycloaddition (more commonly known as “copper-free click”) with appropriately functionalized oligonucleotides as shown in FIG. 21A. In some embodiments, the alkynyl modified oligonucleotides are synthesized by incorporating into the oligonucleotide serinol alkynyl phosphoramidite or DibenzoCyclooctyl (DBCO) phosphoramidite (Glen Research, Inc.). DNA-synbody conjugate are purified by reverse phase HPLC and characterized by MALDI-TOF MS and polyacrylamide gel electrophoresis, and gel shift mobility assays are used to confirm the ability of the DNA handle (oligonucleotide) to bind its complement. In some embodiments, fluorescent dyes are incorporated into the strands to further confirm successful attachment to the DNA origami scaffold. In some embodiments, peptide-DNA conjugates are synthesized using a copper-free click method, as shown in FIG. 21B. In some embodiments, each individual peptide of a successful synbody pair is conjugated to a unique oligonucleotide sequence, the oligonucleotides sequences are complexed together in a direct hybridization or in a DNA nanostructure comprising staple strands, as shown in FIG. 21C.

### Pharmaceutical Compositions

Pharmaceutical compositions of this disclosure may comprise an engineered CAR expressing cells connected to the targeting agent and/or targeting molecule, as described herein, in combination with one or more pharmaceutically or physiologically acceptable carriers, diluents or excipients. Such compositions may comprise buffers including neutral buffered saline, phosphate buffered saline and the like; carbohydrates including glucose, mannose, sucrose or dextrans, mannitol; proteins; polypeptides or amino acids including glycine; antioxidants; chelating agents including EDTA or glutathione; adjuvants (e.g., aluminum hydroxide); and preservatives. Compositions of this disclosure are in one aspect formulated for intravenous administration.

Pharmaceutical compositions of this disclosure may be administered in a manner appropriate to the disease to be treated (or prevented). The quantity and frequency of administration is determined by such factors as the condition of the patient, and the type and severity of the patient's disease, although appropriate dosages may be determined by clinical trials.

Suitable pharmaceutically acceptable excipients can include or exclude phosphate buffered saline (e.g., 0.01 M phosphate, 0.138 M NaCl, 0.0027 M KCl, pH 7.4), an aqueous solution containing a mineral acid salt including a hydrochloride, a hydrobromide, a phosphate, or a sulfate, saline, Ringer's solution, a solution of glycol or ethanol, and a salt of an organic acid including an acetate, a propionate, a malonate or a benzoate. In some embodiments, an adjuvant including a wetting agent or an emulsifier, and a pH buffering agent can also be used. In some embodiments, the pharmaceutically acceptable excipients described in Remington's Pharmaceutical Sciences (Mack Pub. Co., N.J. 1991) (herein incorporated herein by reference) is appropriately used. The composition of this disclosure is formulated into a known form suitable for parenteral administration, for example, injection or infusion. In some embodiments, the composition of this disclosure may comprise formulation additives including a suspending agent, a preservative, a stabilizer and/or a dispersant, and a preservation agent for extending a validity term during storage.

### Methods of Using Engineered T Cells

In some embodiments, a eukaryotic cell expressing the engineered CAR and further comprising the targeting agent and/or targeting molecule is used as a therapeutic agent to treat a disease. The therapeutic agent comprises the eukaryotic cell expressing the expressing the engineered CAR and further comprising the targeting agent and/or targeting molecule as an active ingredient, and may further comprise a suitable excipient. Examples of the excipient include pharmaceutically acceptable excipients for the composition. The disease against which the eukaryotic cell expressing the expressing the engineered CAR and further comprising the targeting agent and/or targeting molecule is administered is not particularly limited as long as the disease shows sensitivity to the eukaryotic cell. Examples of diseases of the invention include a cancer (blood cancer (leukemia), solid tumor etc.).

In some embodiments, the eukaryotic cells with expressing the engineered CAR and further comprising the targeting agent and/or targeting molecule are characterized prior to administration to the subject. In some embodiments, the eukaryotic cells with expressing the engineered CAR and further comprising the targeting agent and/or targeting molecule are tested to confirm the engineered CAR expression. In some embodiments, the eukaryotic cells with expressing the engineered CAR and further comprising the targeting agent and/or targeting molecule are exposed to a level of ligand(s) that results in a desired level of CAR polypeptide expression in the eukaryotic cell. In some embodiments, this desired level of CAR polypeptide produces eukaryotic cells with a desired level of anti-target cell activity, and/or a desired level of proliferative activity when placed in a subject.

In some embodiments, the engineered CAR is used with a T-lymphocyte that has aggressive anti-tumor properties, including those described in Pegram et al, CD28z CARs and armored CARs, 2014, Cancer J. 20(2): 127-133, herein incorporated by reference. In some embodiments, the T-lymphocyte is cultured in a composition comprising high levels of cytokines. The cytokines can include or exclude: IL-2, GMCSF, IL-6, IL-7, IL-4, and IL-15. In some embodiments, the T-lymphocyte is cultured in a composition comprising high levels of chemokine receptors. The chemokine receptors can include or exclude: CCR1, CCR2, CCR4, CCR5, CCR6, CCR7, CCR8, CCR9, CCR10. The chemokine receptors expression level in T cells are monitored by mRNA expression. In some embodiments, chemokine levels are upregulated by culturing the T cells in growth media comprising a cytokine as described herein.

A composition comprising the eukaryotic cells of this disclosure as an active ingredient is administered for treatment of, for example, a cancer (blood cancer (leukemia), solid tumor etc.). The precise amount of the compositions of this disclosure to be administered can be determined by a physician with consideration of individual differences in age, weight, tumor size, extent of infection or metastasis, and condition of the patient (subject). In some embodiments, a pharmaceutical composition comprising the eukaryotic cells described herein may be administered at a dosage of 10\hat{ }4 to 10\hat{ }9 cells/kg body weight, in some instances 10\hat{ }5 to 10\hat{ }6 cells/kg body weight, including all integer values within those ranges. In some embodiments, a eukaryotic cell composition may also be administered multiple times at these dosages. In some embodiments, eukaryotic cells are administered by using infusion techniques.

In some embodiments, the targeting molecule specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof.

In some aspects, the engineered T cells further comprise at least one exogenous protein that modulates a biological effect of interest in an adjacent cell, tissue, or organ, or an exogenous nucleic acid encoding said protein.

### T Cell Therapy

This disclosure provides methods of enhancing the effectiveness of a T cell therapy by adoptive T cell therapy using an engineered T cell comprising an engineered CAR and a targeting agent and/or targeting molecule. In some embodiments, the adoptive T cell therapy can comprise consisting of tumor-infiltrating lymphocyte (TIL) immunotherapy, autologous cell therapy, engineered autologous cell therapy (eACT), allogeneic T cell transplantation, non-T cell transplantation, and any combination thereof. Adoptive T cell therapy broadly includes any method of selecting, enriching in vitro, and administering to a patient autologous or allogeneic T cells that recognize and are capable of binding tumor cells. TIL immunotherapy is a type of adoptive T cell therapy, wherein lymphocytes capable of infiltrating tumor tissue are isolated, enriched in vitro, and administered to a patient. The TIL cells are either autologous or allogeneic. Autologous cell therapy is an adoptive T cell therapy that involves isolating T cells capable of targeting tumor cells from a patient, enriching the T cells in vitro, and administering the T cells back to the same patient. Allogeneic T cell transplantation can include transplant of naturally occurring T cells expanded ex vivo or genetically engineered T cells. Engineered autologous cell therapy, as described in more detail above, is an adoptive T cell therapy wherein a patient's own lymphocytes are isolated, genetically modified to express a tumor targeting molecule, expanded in vitro, and administered back to the patient. Non-T cell transplantation can include autologous or allogeneic therapies with non-T cells including, but not limited to, natural killer (NK) cells.

In one embodiment, the T cell therapy of this disclosure comprises administering an engineered T cell comprising a targeting agent and/or targeting molecule. According to this embodiment, the method can include collecting blood cells from the patient. The isolated blood cells (e.g., T cells) can then be engineered to express an engineered chimeric antigen receptor (“engineered CAR”). In one embodiment, the T cells comprising the engineered CAR are further contacted with a protein tag comprising a first oligonucleotide connected to said protein tag which forms a covalent bond to the adaptor protein on the engineered CAR. The engineered CAR comprising the first oligonucleotide is then contacted with a targeting agent connected to a second oligonucleotide, where a portion of the sequence of the second oligonucleotide is complementary to a portion of the first oligonucleotide and the first and second oligonucleotides form a hybridization duplex. In some embodiments, the targeting agent further comprises a DNA origami nanostructure comprising a plurality of strand sequences. In some embodiments, a first strand sequence comprises a sequence having complementarity to a portion of the first oligonucleotide sequence to form a hybridization duplex. In some embodiments, the first strand sequence comprises a sequence having complementarity to a portion of a first bridging oligonucleotide. In some embodiments, the first oligonucleotide comprises a portion which is complementary to a separate portion of the first bridging oligonucleotide. The first strand, bridging oligonucleotide, and first oligonucleotide can thus form a linked hybridization duplex. In some embodiments, the DNA origami nanostructure can further comprise a second or plurality of distinct strand sequences. The second or plurality of distinct strand sequences can comprise a portion which is complementary to a third oligonucleotide which is connected to a targeting molecule. In some embodiments, the second or plurality of distinct strand sequences can comprise a portion which is complementary to a second bridging oligonucleotide. The third oligonucleotide which is connected to a targeting molecule can comprise a portion which is complementary to a separate portion of the second bridging oligonucleotide. The second or plurality of distinct strand sequences, second bridging oligonucleotide, and third oligonucleotide can form a hybridization duplex.

In one embodiment, the engineered T cells are engineered to express an engineered chimeric antigen receptor. The chimeric antigen receptor can comprise a targeting molecule to a tumor antigen. In some embodiments, the targeting molecule is selected from an antibody or an antigen binding molecule thereof. In some embodiments, the antigen binding molecule is selected from scFv, Fab, Fab′, Fv, F(ab′)2, and dAb, and any fragments or combinations thereof.

The engineered T cell therapy included in this disclosure involves the transfer of T cells, after manipulation of said T cells into engineered T cells, to a patient. The engineered T cells are administered at a therapeutically effective amount. In some embodiments, the therapeutically effective amount of engineered T cells are at least about 10⁴ cells, at least about 10⁵ cells, at least about 10⁶ cells, at least about 10⁷ cells, at least about 10⁸ cells, at least about 10⁹, or at least about 10¹⁰ cells. In some embodiments, the therapeutically effective amount of the engineered T cells is about 10⁴ cells, about 10⁵ cells, about 10⁶ cells, about 10⁷ cells, or about 10⁸ cells. In one particular embodiment, the therapeutically effective amount of the engineered T cells is about 1×10⁵ cells/kg, about 2×10⁵ cells/kg, about 3×10⁵ cells/kg, about 4×10⁵ cells/kg, about 5×10⁵ cells/kg, about 6×10⁵ cells/kg, about 7×10⁵ cells/kg, about 8×10⁵ cells/kg, about 9×10⁵ cells/kg, about 1×10⁵ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg, or between any of the aforementioned T cell amounts. In some embodiments, the therapeutically effective amount of the engineered T cells is about 1×10⁷ cells.

In some embodiments, a therapeutically effective amount of engineered T cells, is from about 1.0×10⁵ cells/kg to about 9×10⁸ cells/kg, or any amount between the aforementioned values.

### T Cell Therapy Employing Saturated Targeting Molecules

In some embodiments, this disclosure provides for methods of engineered T cell therapy by first presenting a targeting agent and/or targeting molecule to the target cancer cell, where the targeting agent and/or targeting molecule comprises a second oligonucleotide, forming a complex between said targeting agent and/or targeting molecule with its cognate ligand on the target cancer cell, then contacting the second oligonucleotide with a engineered T cell comprising a first oligonucleotide where a portion of the sequence of the first oligonucleotide is complementary to a portion of the sequence of the second oligonucleotide. In certain embodiments, this disclosure relates to a method of killing a cancer cell comprising the steps of contacting a cancer cell with a DNA nanostructure comprising a plurality of staple strands where a first staple strand is unhybridized and a second staple strand is hybridized to a fourth oligonucleotide which is chemically conjugated to a targeting molecule, and said targeting molecule binds to said cancer cell; then contacting the DNA nanostructure with an engineered T cell comprising a first oligonucleotide which is complementary to the first staple strand on the DNA nanostructure to form a hybridization complex between the first oligonucleotide and the first staple strand to bring the engineered T cell in local proximity to the cancer cell and begin a cytolytic mechanism.

The inventors have recognized that the advantages of employing a split-step method for T cell therapy are only possible using the engineered T cells of this disclosure. The advantages of employing a split-step method for T cell therapy include: the ability to saturate the targeted cancer cell with a targeting molecule which may be more economical than presenting engineered T cells to target cancer cells first, and that the targeting molecules which are smaller than T cells can penetrate deeper into the target cancer cell than an engineered T cell complex alone. In some embodiments, the targeting agent and/or targeting molecule which is first presented to the target cancer cell comprises a long oligonucleotide linker (e.g., greater than 100 nt, 150 nt, 200 nt, 250 nt, or 300 nt in length or any length between the foregoing nucleotide lengths) which reduces the steric hindrance for the engineered T cells.

In some embodiments, this disclosure includes a composition comprising the components of the split-step method of T cell therapy as described herein. In some embodiments, this disclosure includes a composition comprising: an engineered T cell comprising a first oligonucleotide, and a DNA nanostructure comprising a targeting molecule and a second oligonucleotide, where the first oligonucleotide comprises a portion of which is complementary to a portion of the sequence of the second oligonucleotide. In some embodiments, the engineered T cell comprising a first oligonucleotide is administered simultaneously or sequentially with the DNA nanostructure comprising a targeting molecule. In some embodiments, when the engineered T cell comprising a first oligonucleotide is administered sequentially with the DNA nanostructure comprising a targeting molecule, the two species are administered within 1 hour, 1 day, 1 week, 2 weeks, 3 weeks, or 4 weeks within each other. In some embodiments, when the engineered T cell comprising a first oligonucleotide is administered sequentially with the DNA nanostructure comprising a targeting molecule, the two species are administered within 1 minute, 5 minutes, 10 minutes, 15 minutes, 20 minutes, 25 minutes, 30 minutes, 35 minutes, 40 minutes. 45 minutes, 50 minutes, 55, minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, or any time between the aforementioned values within each other. In some embodiments, when the engineered T cell comprising a first oligonucleotide is administered sequentially with the DNA nanostructure comprising a targeting molecule, the DNA nanostructure is administered locally to a solid tumor site, and the engineered T cell is administered systemically. In some embodiments, when the engineered T cell comprising a first oligonucleotide is administered sequentially with the DNA nanostructure comprising a targeting molecule the engineered T cell and DNA nanostructure comprising a targeting molecule are administered at or about at the same location in the subject. In some embodiments, the DNA nanostructure comprising a targeting molecule is administered at a dose of about 0.001 mg/kg to about 5 g/kg to the subject, or any dose level between the aforementioned values.

In some embodiments, the targeted cancer cell is any of the cancer cell types described herein.

### Cancer Treatment

This disclosure provides for methods treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In some embodiments, the methods induce a complete response. In some embodiments, the methods induce a partial response.

Cancers that may be treated include tumors that are not vascularized, not yet substantially vascularized, or vascularized. The cancer may also include or exclude solid and non-solid tumors. In some embodiments, the cancer is selected from a tumor derived from bone cancer, lung cancer, brain cancer, breast cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the endometrium, carcinoma of the fallopian tubes, carcinoma of the cervix, carcinoma of the vagina, carcinoma of the vulva, Hodgkin's Disease, T-cell rich B cell lymphoma (TCRBCL), Primary mediastinal large B cell lymphoma (PMBCL), non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, primary CNS lymphoma, neoplasm of the central nervous system (CNS), tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In some embodiments, the methods described here are used to treat a tumor, wherein the tumor is a lymphoma or a leukemia. Lymphoma and leukemia are cancers of the blood that specifically affect lymphocytes. The blood cancers which are treated can include or exclude cancers of T lymphocytes (T cells), B lymphocytes (B cells), natural killer cells, and plasma cells. Leukocytes arising from the lymphoid progenitor cells include megakaryocytes, mast cells, basophils, neutrophils, eosinophils, monocytes, and macrophages. Lymphomas and leukemias can affect one or more of these cell types in a patient.

In some embodiments, the methods described herein are used to treat a lymphoma or a leukemia, wherein the lymphoma or leukemia is a B cell malignancy. In some embodiments, the lymphoma or leukemia is selected from B-cell chronic lymphocytic leukemia/small cell lymphoma, B-cell prolymphocytic leukemia, lymphoplasmacytic lymphoma (e.g., Waldenström macroglobulinemia), splenic marginal zone lymphoma, hairy cell leukemia, plasma cell neoplasms (e.g., plasma cell myeloma (i.e., multiple myeloma), or plasmacytoma), extranodal marginal zone B cell lymphoma (e.g., MALT lymphoma), nodal marginal zone B cell lymphoma, follicular lymphoma (FL), transformed follicular lymphoma (TFL), primary cutaneous follicle center lymphoma, mantle cell lymphoma, diffuse large B cell lymphoma (DLBCL), Epstein-Barr virus-positive DLBCL, lymphomatoid granulomatosis, primary mediastinal (thymic) large B-cell lymphoma (PMBCL), Intravascular large B-cell lymphoma, ALK+ large B-cell lymphoma, plasmablastic lymphoma, primary effusion lymphoma, large B-cell lymphoma arising in HHV8-associated multicentric Castleman's disease, Burkitt lymphoma/leukemia, T-cell prolymphocytic leukemia, T-cell large granular lymphocyte leukemia, aggressive NK cell leukemia, adult T-cell leukemia/lymphoma, extranodal NK/T-cell lymphoma, enteropathy-associated T-cell lymphoma, Hepatosplenic T-cell lymphoma, blastic NK cell lymphoma, Mycosis fungoides/Sezary syndrome, Primary cutaneous anaplastic large cell lymphoma, Lymphomatoid papulosis, Peripheral T-cell lymphoma, Angioimmunoblastic T cell lymphoma, Anaplastic large cell lymphoma, B-lymphoblastic leukemia/lymphoma, B-lymphoblastic leukemia/lymphoma with recurrent genetic abnormalities, T-lymphoblastic leukemia/lymphoma, and Hodgkin lymphoma. In some embodiments, the cancer is refractory to one or more prior treatments, and/or the cancer has relapsed after one or more prior treatments.

In some embodiments, the cancer is selected from follicular lymphoma, transformed follicular lymphoma, diffuse large B cell lymphoma, and primary mediastinal (thymic) large B-cell lymphoma. In some embodiments, the cancer is diffuse large B cell lymphoma.

In some embodiments, this disclosure includes a method of treating a patient having a lymphoma comprising administering to the patient a therapeutically effective amount of engineered T cells which further comprise a targeting agent and/or targeting molecule, and wherein the targeting molecule binds to CD19 and the engineered T cells further comprises a CAR which expresses a CD28 costimulatory domain and a CD3-zeta signaling region.

When used for immunotherapy applications, T-cells are removed from a patient through leukopheresis and T-cells are preferentially sorted and saved. T cells are subjected to lentiviral or retroviral introduction (or other means of nucleic acid introduction) of the transgene that encodes the engineered CAR as described herein. The engineered T cells with the expressed engineered CAR are then contacted with the targeting first oligonucleotide comprising a protein tag, then contacted with a targeting agent connect to a second oligonucleotide which comprises a portion which is complementary to the sequence of the first oligonucleotide. The engineered T cells comprising the targeting agent are then expanded to target therapeutic cell concentrations and infused into the patient, resulting in an autologous treatment with minimal graft to host complications.

In some embodiments, the engineered T cell comprising a targeting molecule and/or agent is engineered to target a particular cancer cell antigen. In some embodiments, the cancer cell antigen is selected from CD19 CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated Ras, HER2/Neu, folate binding protein, HIV-1 envelope glycoprotein gp120, HIV-1 envelope glycoprotein gp41, GD2, BCMA, IgK, Lewis Y, CD123, CD33, CD138, CD23, CD30, CD56, c-Met, mesothelin, GD3, HERV-K, IL-11Ralpha, kappa chain, lambda chain, CSPG4, ERBB2, EGFRvlll, PSMA, MUC1, L1-CAM, IL13Ra2, Fr-a, CEA, CAIX, VEGFR2, HER2-HER3 in combination, HER1-HER2 in combination, and any combination thereof. In one particular embodiment, the cancer cell antigen is CD19. FIG. 30A shows the present clinical outcomes of published attempts at CAR T cell therapy for hematological malignancies, indicating that T cells targeted to CD19 have the highest rate of complete response or positive response for hematological malignancies. FIG. 30B shows the present clinical outcomes of other attempts at CAR T therapy for solid tumor therapies, indicating that T cells targeting GD2 have the highest complete response or positive response for solid tumor malignancies.

In some embodiments, the engineered T cell comprises a targeting molecule to a testicular, placental, or fetal tumor antigen. In one embodiment, the testicular, placental, or fetal tumor antigen is selected from the group consisting of NY-ESO-1, synovial sarcoma X breakpoint 2 (SSX2), melanoma antigen (MAGE), and combinations thereof.

In some embodiments, the engineered T cell comprises a targeting molecule to a lineage specific antigen. In one particular embodiment, the lineage specific antigen is selected from the group consisting of melanoma antigen recognized by T cells 1 (MART-1), gp100, prostate specific antigen (PSA), prostate specific membrane antigen (PSMA), prostate stem cell antigen (PSCA), and combinations thereof.

In some embodiments, the engineered T cell therapy comprises administering to the patient engineered T cells comprising a targeting molecule and/or agent that binds to CD19, and further comprises a CD28 costimulatory domain and a CD3-zeta signaling region.

### Kits

This disclosure includes pharmaceutical kits, comprising engineered T cells for engineered T cell therapy. Kits can include or exclude a label indicating the intended use of the contents of the kit and instructions for use. The term “label” includes any writing, or recorded material supplied on or with the kit, or which otherwise accompanies the kit. Kits can include or exclude a pharmaceutically acceptable carrier as described herein. The pharmaceutically acceptable carrier can include or exclude saline, Ringer's solution, a citrate, a phosphate, PBS, and any other carrier as described herein.

The invention will now be illustrated by the following non-limiting Examples.

### EXAMPLES

Within this application, unless otherwise stated, the techniques utilized may be found in any of several well-known references including: Molecular Cloning: A Laboratory Manual (Sambrook, et al., 1989, Cold Spring Harbor Laboratory Press), Current Protocols in Molecular Biology (Ausebel et al., Wiley-Interscience, 1988. New York), and PCR Protocols: A Guide to Methods and Applications (Innis, et al. 1990. Academic Press, San Diego, Calif.), each of which is herein incorporated by reference.

### Example 1: Designing Engineered T Cells to Target Solid Tumors

Engineered T cell therapy involves the use of a T cell with an engineered CAR which comprises an adaptor protein which is connected to a protein tag, an oligonucleotide, and a targeting agent, a transmembrane spacer, and an intracellular signaling/activation domain(s). The engineered T cells are transfected into T cells using plasmid transfection, mRNA, or viral vector transduction to direct the cells toward surface-exposed tumor-associated antigens (TAAs). The CAR structure has evolved from an initial composition involving only the CD3ζ signaling domain (dubbed a “first-generation CAR”) to more complex forms in which costimulatory endodomains have been added, giving rise to second-generation (e.g., CD3ζ plus 41BB or CD28 signaling domains) and third-generation (e.g., CD3ζ plus 41BB and CD28 signaling domains) CARs that have augmented T cell persistence and proliferation.

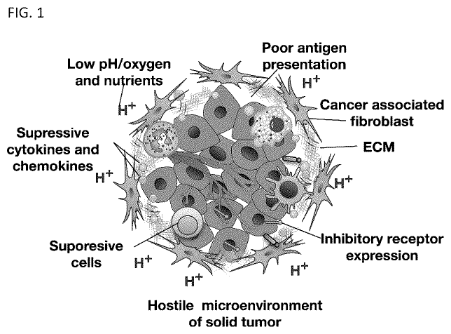

The adoptive transfer of T cells to solid tumors presents difficulties including physical barriers that are absent in hematologic malignancies. Discovery of specific tumor antigens that are highly and uniformly expressed has been rare. Unlike hematologic malignancies, tumor cell malignancies require T cells to successfully traffic from the blood into solid tumor sites in spite of potential T cell chemokine receptor-/tumor-derived chemokine mismatches. The T cells must then successfully infiltrate the stromal elements of solid tumors to elicit TAA-specific cytotoxicity, regardless of antigen loss or heterogeneity. Even after successful trafficking and infiltration, T cells become rapidly dysfunctional owing to 1) a hostile tumor microenvironment (TME) characterized by oxidative stress, nutritional depletion, acidic pH, and hypoxia; 2) the presence of inhibitory soluble factors and cytokines; 3) suppressive immune cells, namely regulatory T cells (Tregs), myeloid-derived suppressor cells (MDSCs), and tumor-associated macrophages (TAMs) or neutrophils (TANs); and 4) T cell-intrinsic negative regulatory mechanisms, including upregulation of cytoplasmic and surface inhibitory receptors programmed cell death protein 1 (PD-1) and cytotoxic T-lymphocyte-associated antigen 4 (CTLA4) (FIG. 1).

The clinical results of using T cell therapy in solid tumors have been unsuccessful due to the hostile microenvironment of solid tumors, including physical matrix barriers, antigen loss, co-inhibitory molecules and inhibitory cytokines. The present disclosure includes a novel DNA nanostructure-based strategy to further engineer T cells to: 1) Precisely target tumor cells through dual/multiple-binding specificities to minimize tumor immune escape; 2) Increase cell survival and trafficking to tumor area; and 3) Generate multifunctional engineered T cells rapidly. A novel engineered T cell toolkit is generated using DNA nanotechnology (“engineered T cells”). Engineered T cells are designed to express signaling CAR domains and further comprise an “adaptable receptor” protein (also referred to as an “adaptor protein”) that enables the attachment of a targeting agent. In some embodiments, the targeting agent comprises a DNA origami nano-scaffold (“DNA nanostructure”). These DNA nanostructures comprise targeting molecules which can include or exclude peptides, synbodies, aptamers, and small molecules. In some embodiments, the targeting molecules include additional functionality including cytokines and matrix metallopeptidases (MMPs) to enhance T cell survival and their penetration infiltration into the tumor environment of solid tumors. The inventors were the first to recognize that this highly modular and combinatorial approach circumvents the issues with genetic engineering, enabling non-genetically encodable functionality, and enhance engineered T cell activity through multivalent effects. The final engineered T cells significantly improve the application of engineered T cell therapy to treating solid tumors.

This disclosure provides for a solution to tumor cell targeting using engineered T cells without the use of long-term molecular cloning and structure optimizations.

The inventors have surprisingly discovered a novel method to engineer T cells to include a variety of, and population of, targeting agents and/or targeting compounds which enables modulation for optimal cancer cell targeting.

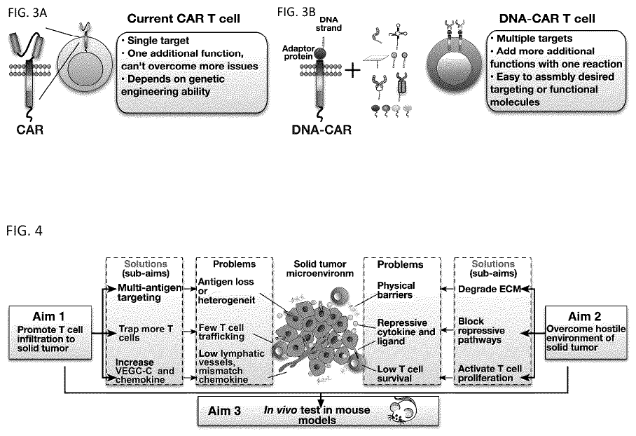

Engineered T cell immunotherapy for solid tumor cancers overcomes problems including low level or loss of tumor antigen expression, poor T cell trafficking and infiltration into the tumor, and an immune response-repressive environment (FIG. 1). Using DNA, RNA and/or peptide nanotechnologies, engineered T cells are provided that target cancer cells (e.g., tumor cells) with remarkable specificity for different levels of expression, have increase survival resistance to the immunosuppressive solid tumor environment, and increase T cell traffic to the tumor area. This method also allows for low-cost and rapid methods of generating multifunctional engineered T cells without further genetic engineering. In some embodiments, the T cell is engineered to comprise a targeting molecule specific for different cancer types, stages and cancer antigen expression of individual patients. In some embodiments, the novel DNA nanotechnology-based strategy to engineer T cells disclosed herein can: 1) allow for precise tumor targeting through dual/multiple-binding specificities to minimize tumor immune escape; 2) increase cell survival and trafficking to tumor area; and/or 3) generate multifunctional engineered T cells rapidly and at low cost. T cells are engineered that express signaling CAR domains and an “adaptable receptor” protein domain (also referred to herein as “adaptor protein”) that enables the attachment of a DNA origami nanostructure with high efficiency (FIG. 3B). These DNA nanostructures can carry targeting molecules which can include or exclude peptides, synbodies, aptamers, or small molecules. In some embodiment, the DNA nanostructures can carry and/or present functional molecules to enhance T cell survival and penetration into the solid tumor environment.

Conventional CAR T therapy on solid tumors has not been successful due to the hostile microenvironment of solid tumors, including physical barriers presented by the extracellular matrix (ECM), loss of the targeted antigen, or the presence of co-inhibitory molecules and inhibitory cytokines (FIG. 3A).

In some embodiments, the approach for engineering T cells is presented in FIG. 3B, where one single T cell expressing a CAR domain is engineered with a protein “adaptor” that allows the attachment of a targeting agent comprising DNA origami nano-scaffold to the cell surface using a single, reliable chemistry. In some embodiments, the adaptor protein is a protein which has the ability to form a covalent bond with a small molecule, where the small molecule may be covalently linked to an oligonucleotide. In some embodiments, the adaptor protein is a SNAP-Tag™, CLIP-Tag™, or HALO-Tag™ protein.

This disclosure provides for methods and compositions for overcoming challenges in T cell therapy targeting solid tumors: low T cell infiltration (as shown in FIG. 4, left “Aim 1”), and low T cell activity (as shown in FIG. 4, right, “Aim 2”). By dissecting these challenges, a series of solutions are provided that are unified under a common engineered T cell. The first challenge that allows tumor cells to escape T cell recognition is antigen loss and or heterogeneity. To address this challenge, peptide-based multivalent synbodies, antibodies or aptamers targeting cancer surface biomarkers broadly expressed in different cancer types are screened. The antigens can include or exclude any of the antigens in Table 2. These T cells carry targeting molecules—including peptides, aptamers, and/or proteins—allowing for a highly modular system that can target any desired cancer, without the requirement for genetic surface engineering of the T cell. In some embodiments, the targeting agent is DNA origami (also referred to herein as “DNA nanostructures” or “DNA origami nanostructures”) which yields highly programmable structures and can used for attracting and trapping circulating T cells and/or targeted delivery of T cell recruiting chemokines. These DNA nanostructures can comprise targeting molecules for addressing the issues inherent with targeting solid tumors. In some embodiments, the targeting molecule is selected from an extracellular matrix (ECM)-degrading matrix metallopeptidases (MMPs), a blocking repressive pathway, or activating T cell proliferation.

In some embodiments, the engineered T cell is tested in an adoptive transfer mouse model to obtain the best combinations of functional targeting molecules. The inventors have discovered that the use of DNA origami allows for rapid prototyping of ligand combinations with multivalency, control over nanoscale spacing, and incorporation of non-genetically encodable components like imaging agents, small molecules, or peptides with non-canonical amino acid functionality. These modular multifunctional engineered T cells synergistically improve the application of engineered T cell therapy in treating in solid tumors.

TABLE 2
Engineered T cell antigen candidates for solid tumors

| Antigen | Type of cancer |
|---|---|
| CD133 | Liver, brain, breast, AML, ALL |
| CEA | Lung, colorectal, gastric, pancreatic Colorectal, |
| | adenocarcinoma Liver metastases |
| EGFR | Lung, colorectal, ovary, pancreatic Advanced glioma |
| | Malignant glioma, glioblastoma |
| GD2 | Sarcoma, osteosarcoma, neuroblastoma, melanoma, |
| | Relapsed/refractory Neuroblastoma Metastatic |
| | melanoma |
| GPC3 | Lung squamous cell carcinoma |
| HER2 | Glioblastoma multiforme, Her+ cancers Breast, |
| | ovarian, lung, pancreatic, gastric, HCC, endometrial, |
| | refractory to chemotherapy and Her2 antibody, |
| | Advanced sarcoma |
| Mesothelin | Pancreatic adenocarcinoma, ovarian cancer, |
| | malignant epithelial pleural mesothelioma, |
| | Malignant mesothelioma, Metastatic Her2- breast, |
| | Glioma, colorectal carcinoma, gastric carcinoma |

This disclosure provides for methods of promoting T cell infiltration in solid tumors by DNA nanostructure to demonstrate feasibility of using engineered T cells to kill cancer cells and to activate engineered T cells in the presence of a cancer cell.

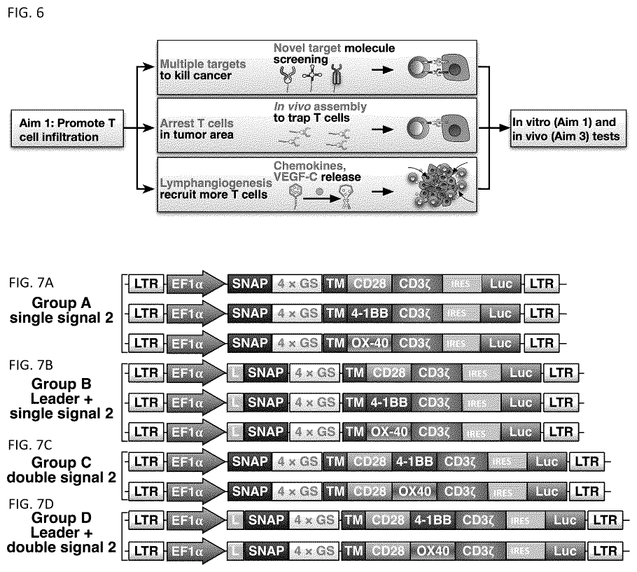

Solid tumors present a unique therapy challenge due to growth and the interaction with cells associated with the tumor cell microenvironment. The competition for nutrients and mismatches in chemokines resist T cell infiltration from circulations. Also, lymphangiogesis blocks T cells from getting into the tumor cell area. High mutation rates that cause antigen loss or heterogeneity also help solid tumor cells escape killing by T cells. The inventors have surprisingly discovered a unique design to address these issues by engineering an T cell comprising one or a plurality of targeting agent comprising a DNA nanostructure which comprises targeting molecules on or about at the T cell surface. By using the adaptor protein connected to a first oligonucleotide which has a portion of the sequence is complementary to a portion of a second oligonucleotide, where the second oligonucleotide is a staple strand in a DNA nanostructure, the adaptor-CAR expressing T cells can add multiple types of targeting molecules to the T cell surface which is presented to the tumor antigen. In some embodiments, the DNA nanostructure is a DNA nanocage. The DNA nanocage can open upon a stimulus to present a biomolecule. In some embodiments, the biomolecule is a chemokine or VEGF-C, which can build lymphic vessels for T cells to increase T cell recruiting, as shown in FIG. 6.

This disclosure provides for methods to trap engineered T cells in solid tumor by DNA nanostructure to demonstrate feasibility of using engineered T cells to kill cancer cells and to activate engineered T cells in the presence of a cancer cell.

In some embodiments, as an alternative to attaching a pre-formed DNA nanostructure comprising targeting ligands to the engineered T cell, and allowing the cells to circulate and bind to the tumor, a two-step process is performed: first, a DNA nanostructure comprising targeting molecules (e.g., synbodies, aptamers) is injected and allowed to circulate, accumulating in the tumor area. This DNA nanostructure comprises additional DNA staple strands, thereby effectively displaying addressable “handles” on the tumor. In a second step, engineered T cells comprising staple strands which comprise complementary DNA handles are injected, allowing them to accumulate in the tumor region through DNA hybridization. This method effectively decouples the nanostructure binding to the tumor from the engineered T cell modification. The method also enables for enhanced multivalency in both the targeting and cell modification steps.

The DNA nanostructures used to trap engineered T cells in solid tumor comprises a plurality of unique staple strands, where each staple strand presents a unique “handle” to trap the engineered T cells comprising a first oligonucleotide which is complementary to the sequence of the “handle” sequence in circulation. The inventors have recognized that a key advantage of DNA origami targeting structures is the facile incorporation of a plurality of DNA handles (staple sequences) to significantly enhance subsequent cell binding; in effect, a single recognition event in the tumor results in the display of many identical DNA handles. In some embodiments, the DNA nanostructures present a dense array of 10-15 nt handles, as shown in FIG. 13. In some embodiments, the DNA nanostructure comprises a dendrimeric architecture, assembled from multiple generations of Y-shaped branched DNA monomers, as shown in FIG. 13A, allowing for a plurality (e.g., 8-16) handles per targeting structure. In some embodiments, the DNA nanostructure further comprises a self-assembled linear repeating nanoribbons, as shown in FIG. 13B, which is modulated to display a plurality of identical ssDNA handles (staple strands). In some embodiments, staple strands for each approach is mixed in equimolar ratios and annealed beginning from about 95° C. down to about 4° C. for several hours (e.g., 1 to 8 hours, in some embodiments, the annealing time is 1 hour, 2 hours, 3, hours, 4 hours, 5 hours, 6 hours, 7 hours, 8 hours, or any time between the aforementioned times) to anneal them, followed by purification using polyacrylamide or agarose gel electrophoresis. In some embodiments, the annealing temperature begins at about 97° C., 96° C., 95° C., 95° C., 93° C., 92° C., 91° C., 90° C., 89° C., 88° C., 87° C., 86° C., 85° C., 84° C., 83° C., 82° C., 81° C., 80° C., or any temperature between the aforementioned temperatures. In some embodiments, the start annealing temperature is set to be above the highest melting temperature (“Tm”) of the complementary oligonucleotide sequences in the DNA or RNA nanostructure, but below that of the boiling point of the aqueous solution in which the oligonucleotides are dissolved. In some embodiments, the annealing temperature can end at any temperature below the lowest melting temperature (“Tm”) of the complementary oligonucleotide sequences in the DNA or RNA nanostructure, but higher than the freezing temperature of the aqueous solution in which the oligonucleotides are dissolved. In some embodiments, the annealing temperature can end at about 15° C., 14° C., 13° C., 12° C., 11° C., 10° C., 9° C., 8° C., 7° C., 6° C., 5° C., 4° C., 3° C., 2° C., or 1° C. In some embodiments, one side of a DNA origami substrate is modified with a plurality of staple strands (e.g., 10-100) by simply extending staple strands with a constant single-stranded region. In some embodiments, tumor localization is assessed using fluorescently labeled nanostructures (via incorporation of fluorophore-modified oligonucleotides into the annealing protocol).

In some embodiments, the engineered T cells are contacted with an adaptor protein (e.g., SNAP-Tag™) which is connected to an oligonucleotide which is hybridized to a branching DNA structure. In some embodiments, the branching DNA structure is a multivalent structure comprising series of oligonucleotides which comprise a sequence of complementary sequences to other branching oligonucleotides, each of which is connected to a protein tag (e.g., benzylguanine residue). In some embodiments, methods of this disclosure include presenting DNA nanostructures comprising branching DNA structures to tumor cells to decorate both the tumor and the targeting cells with a dense brush of DNA comprising protein tags, akin to Velcro, to significantly enhance engineered T cell binding. A standard trans-well assay measures whether engineered T cells are arrested by tumor cells using the methods described herein. Cell modification strand sequences is labeled with a different fluorescent dye to probe co-localization between the tumor and the cells.

This disclosure provides for smart DNA nanostructures to increase engineered T cells in tumor area to demonstrate feasibility of using engineered T cells to kill cancer cells and to activate engineered T cells in the presence of a cancer cell.

Chemokines are essential coordinators of cellular migration and cell-cell interactions, and therefore have significant impact on tumor development. In the tumor microenvironment (TME), tumor-associated host cells and cancer cells release an array of different chemokines, resulting in the recruitment and activation of different cell types that mediate the balance between antitumor and pro-tumor responses. In addition to their primary role as chemo-attractants, chemokines are also involved in other tumor-related processes, including tumor cell growth, angiogenesis, and metastasis. Chemokines play an important role in leukocyte infiltration into any tissue, including tumors. Hence, they have a critical role shaping the immune cell composition in the TME, which affects tumor development. Immune cells that are responsible for the removal of cancer cells are called “effector cells,” which are able to kill or control the growth of tumor cells. Many human tumors produce low levels of chemokines, while others produce chemokines for which effector T cells lack receptors. As a consequence, adoptively transferred T cells may simply fail to “find” the tumors. In some embodiments, methods of this disclosure include enhancing engineered T cell trafficking by increasing the expression of the chemokine receptor CCR2 is performed to reduce the residual chemokine levels.

In some embodiments, the DNA nanostructures are DNA nanocages comprising a therapeutic agent. DNA nanocages are comprised of DNA structures which are shaped to contain a delivery agent. In some embodiments, the delivery agent is a therapeutic agent as described herein. In some embodiments, the therapeutic agent is a targeting molecule.

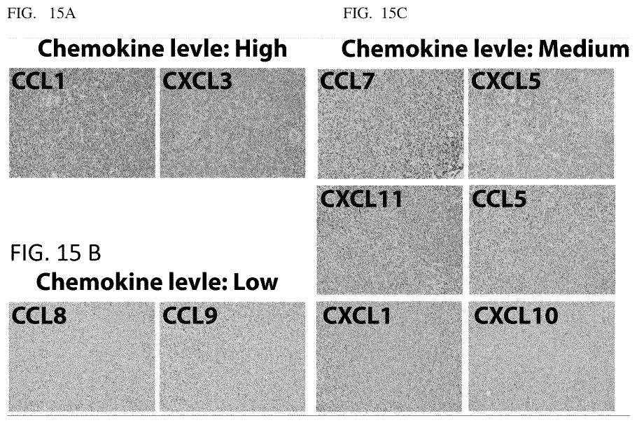

Only a few antigen-specific T cells infiltrate breast cancer tissue. Different chemokines expression levels in MDA-MB-231 breast cancer tumor were measured by immunohistochemistry (IHC) staining, as shown in FIG. 15. CCL1, which is the ligand of CCR8 and CXCL3, which is the ligand of CXCR3 expression, are high in MDA-MB-231 breast cancer tumors; however, the receptor of CCL1, CCR8, is low in antigen-specific T cells. In addition, T cells express high levels of CCR2, which is the receptor of CCL7 and CCL8. CCL7 and CCL8 expression is low in tumor tissue. The mismatch of chemokines from tumor cells and chemokine receptors on antigen specific T cells is one reason for low T cell infiltration.

### Design and Demonstration of a Model Nucleolin-Responsive DNA Nanocage Opening to Present Therapeutic Agents in the TME

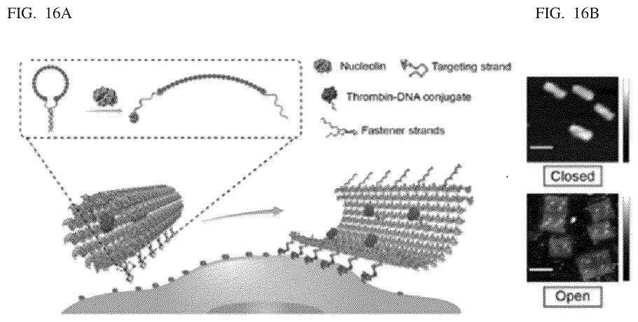

In some embodiments, the DNA nanocage (also referred to as “DNA origami nanocage”) comprises thrombin for delivery to tumor-associated blood vessels to block the tumor blood supply. In some embodiments, the DNA nanocage is that described U.S. patent application Ser. No. 16/208,103, herein incorporated by reference. In some embodiments, the DNA origami nanocage comprises two aptamers to nucleolin along opposite sides of the periphery of a DNA nanostructure which is in the form of a flat sheet. In the presence of nucleolin, the aptamers bind to nucleolin bringing the periphery of the DNA nanostructure together to form a tube. The two aptamers bound to the nucleolin form the cage seam. In some embodiments, the therapeutic agent is thrombin. In some embodiments, thrombin molecules is attached at designated positions inside the DNA cage, with the aptamer nucleolin displayed on the outside of said cage. When the aptamers recognize their target nucleolin, a protein highly expressed on the surface of tumor vascular endothelia, the hybridized duplexes of the fastening strands dissociate, and the DNA nanocage undergoes a massive reconfiguration to expose its inside surface and the bound thrombin molecules causing tumor infarction, as shown in FIG. 16.

In some embodiments, the therapeutic agent is selected from chemokines and VEGF-C.

In some embodiments, the structure of the DNA nanocages is designed using the DNA origami method, whereby a long circular scaffold DNA strand folds itself into a prescribed shape with the hybridization of a plurality of short (e.g., 12-25 nt in length) staple DNA strands (“staple strands”). Design parameters, including the inner cage volume and cage lock position and number is tailored to achieve a desired loading capacity and lock/unlocking efficiency. In some embodiments, the constructed DNA nanocages are purified with Amicon columns or gel electrophoresis to remove excess staple strands. Final structures are imaged with transmission electron microscopy (TEM) to validate their 3D structural features. The basic DNA nanocage is functionalized with the following three functional modules. First, the nanocage is modified with a nucleolin aptamer lock. In some embodiments, the aptamer is chemically modified with PS2 or MS2 modifications to achieve better biological stability. In some embodiments, the therapeutic agent is a chemokine molecule which is used to recruit circulating T cells after being released from DNA nanocage. In some embodiments, the therapeutic agent is VEGF to increase lymphatic vessels in solid tumors. In some embodiments, both a plurality of different chemokine molecules is used to synergistically kill tumor cells.

In some embodiments, the DNA nanostructure is functionalized by modifying selected staple strands with polyethylene glycol (PEG) to increase stability during circulation.

This disclosure provides for methods of overcoming the hostile solid tumor microenvironment using engineered T cells to establish the enablement of using engineered T cells to kill cancer cells and to activate engineered T cells in the presence of a cancer cell.

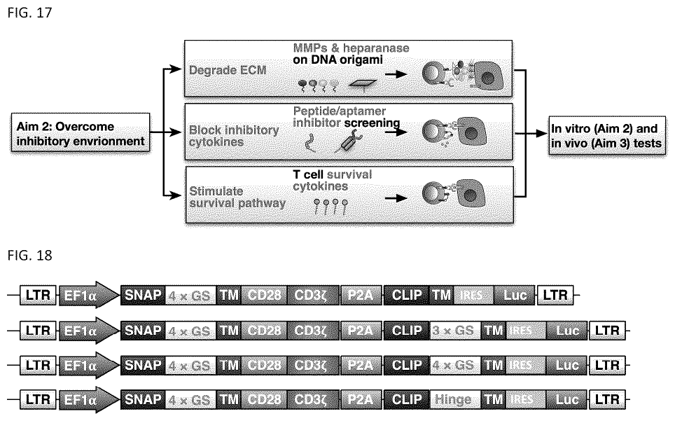

The solid tumor microenvironment blocks killing by T cells with both physical barriers like the extracellular matrix (ECM), high levels of repressive cytokines including IL-10 and TGFbeta, and low levels of the proliferation cytokine IL-2. Furthermore, once T cells have entered the tumor area, they can be repressed by inhibitory ligands including PD-L1. The inventors have discovered that engineered T cells comprising engineered CAR constructs which encode a second adaptor protein (e.g., CLIP-Tag™), allows for conjugation to additional targeting molecules on the engineered T cell surface. Besides expressing more adaptor proteins on the T cell, the use of DNA nanostructures enables conjugation of a plurality of types of proteins, peptides or aptamers to the engineered T cell, as shown in FIG. 17 using the CAR constructs depicted in FIG. 18. In some embodiments, the engineered T cells comprises a targeting molecule which is a matrix metallopeptidases (MMPs) or heparanase, to degrade tumor ECM. In some embodiments, the targeting molecule is a peptide or aptamer to block inhibitory cytokine pathways of T cells. In some embodiments, the targeting molecule links cytokines to the engineered T cell surface to increase the engineered T cell survival rate.

### Targeting ECM in Solid Tumors

This disclosure provides for methods of degrading ECM by connecting DNA conjugated functional molecules to establish enablement of using engineered T cells to kill cancer cells and to activate engineered T cells in the presence of a cancer cell.

Tumor fibroblasts and myeloid cells are responsible for the development of a fibrotic extracellular matrix (ECM), which may impede T cell penetration. The enzyme heparinase (HPSE) can disintegrate heparan sulphate proteoglycans, which largely constitutes the ECM. However, in vitro-cultured non-modified T cells do not express HPSE. In some embodiments, the engineered T cells comprise an engineered CAR which expresses HPSE to degrade the ECM resulting in improved engineered T-cell infiltration and antitumor activity.

In some embodiments, the targeting molecule is hybridized to the DNA nanostructure by chemically conjugating the targeting molecule with an oligonucleotide which comprises a sequence of which a portion is complementary to a portion of the staple strand on the DNA nanostructure. In some embodiments, the targeting molecule is chemically conjugated to the oligonucleotide using a heterobifunctional linker (e.g., bis-NHS esters).

The inventors have discovered a strategy to synthesize targeting molecule-DNA conjugates so as to not occlude the functional regions—the catalytic site responsible for matrix degradation for MMPs, and the receptor binding face of cytokines like IL-2, which need to bind to hetero-trimeric receptors on the engineered T cell surface—of said targeting molecules. Where the targeting molecule is selected from a MMP or proliferative cytokines, a strategy to not occlude the functional regions of these proteins is employed, as show in FIG. 21D. In some embodiments, a unique reactive surface amino acid including cysteine in the protein (for expression in E. coli), is introduced for modification with a maleimide-modified DNA, as shown in FIG. 21E. In other embodiments, the peptide sequence LPETG (SEQ ID NO: 12) is introduced into a terminus or loop of the targeting molecule proteins, which is then modified after expression using the enzyme sortase, with a DNA-(G)5 conjugate, as shown in FIG. 21F.

TABLE 3
Molecule candidates for bioconjugation

| Molecules | Functions | Name |
|---|---|---|
| Proteins | ECM degrade | MMP2 |
| | | MMP9 |
| | | heparanase |
| | T cell recruiting | CCL7 |
| | | CCL8 |
| | | VEGF-C |
| | T cell survival | IL-2 |
| | | IL-7 |
| | | IL-15 |
| | | IL-21 |
| Peptide | Targeting synbody | CD133 |
| | | CEA |
| | | EGFR |
| | | GD2 |
| | | GPC3 |
| | | HER2 |
| | | Mesothelin |
| | Inhibitory synbody | PD-1 |
| | | PD-L1 |
| | | TGFβ |
| | | TGFβR |
| | | CTLA4 |

In some embodiments, the targeting molecule is a protein or peptide listed in Table 2.

In some embodiments, the protein/peptide-DNA conjugates are purified by anion-exchange and/or size-exclusion chromatography and optionally characterized by polyacrylamide gel electrophoresis. In some embodiments, the functionality of the DNA handle is probed by hybridization to its complementary oligonucleotide strand and analyzed by mobility gel shift assay. The protein/peptide-DNA conjugates are immobilized on DNA nanostructures by hybridization with their cognate staple strand sequences. In some embodiments, the functionality of MMPs on the DNA nanostructures is confirmed via a matrix degradation assay. In some embodiments, the functionality of cytokines is confirmed using receptor binding studies.

In some embodiments, the engineered T cells comprise a targeting molecule which is an attached matrix metallopeptidases (MMPs) to the DNA region of the T cell CAR to degrade tumor extracellular matrix (ECM), where the targeting molecule is immobilized to the DNA nanostructure (“MMP-DNA-CAR-T cell”). In some embodiments, the engineered T cells comprise a targeting molecule which is an attached heparinase (HPSE) to the DNA region of the T cell CAR to degrade tumor extracellular matrix (ECM), where the targeting molecule is immobilized to the DNA nanostructure (“HPSE-DNA-CAR-T cell”). The capacity of each type of engineered T cell subset to degrade ECM is examined in vitro using the BioCoat Matrigel Invasion assay (Becton Dickinson Biosciences). Data is expressed as the percentage of invasion through the Matrigel and the membrane relative to the migration through the control membrane (8 μm polyethylene terephthalate membrane pores). The percentage of invasion is calculated as follows: (mean of cells invading through the Matrigel chamber membrane/mean of cells migrating through the control insert membrane)×100. The invasion and antitumor activity of MMP-DNA-CAR-T cell or HPSE-DNA-CAR T cells is determined using the BioCoat Matrigel Invasion assay by plating MMP-DNA-CAR-T cells (1.4×10⁵) in the bottom of a 24-well plate with MMP-DNA-CAR-T cell and H-DNA-CAR T cells. 2.5×10⁶ cells are placed in the upper chamber/insert. Chambers and inserts are removed 24 h later. After 3 d of culture cells are then collected from the lower chamber and quantified by flow cytometry to identify tumor cells and T cells, respectively. Multiplex analysis for matrix metalloproteases (MMPs) analysis is performed using Milliplex Map kit panel 1 and 2 (Millipore) according to the manufacturer's instructions. In particular, FI-T cells is activated with OKT3 and anti-CD28 mAbs in presence of IL-2(50 U/mL or 2000 U/mL) or IL-7 and IL-15 (10 ng/ml and 5 ng/ml, respectively). The engineered T cells are fed twice a week. At days 3, 7 and 10 of culture, supernatants and T cells are collected and 60 μg of sample is tested per well. The protein-DNA conjugates which bind to DNA nanostructures are optimized to retain the function of the ECM-degrading protein.

### Blocking T Cell Inhibitory Pathways to Target Solid Tumors

In some embodiments, this disclosure provides methods of blocking T cell inhibitory pathways in solid tumors by synbodies or DNA aptamers to show enablement of using engineered T cells to kill cancer cells and to activate engineered T cells in the presence of a cancer cell.

Checkpoint inhibitory proteins are often upregulated on tumors, including PD-L1 with the normal function of modulating immune response. Once PD-L1 binds to its inhibitory receptor PD-1 on T cells, the function of T cells is inhibited; in other words, T lymphocytes can become hypofunctional.

The tumor microenvironment (TME) comprises numerous types of suppressive immune cells and molecular factors which inhibit T cells anti-tumor immune function within the TME. Thus, T lymphocytes must overcome tremendous challenges to exert effective antitumor activity, including immune suppressor cells including Tregs, myeloid-derived suppressor cells (MDSCs), and tumor-associated macrophages (TAMs); cytokines and soluble factors associated with immunosuppression, including TGF-β and IL-10; and checkpoint inhibitory proteins, including PD-L1 and/or CTLA4. The inventors have recognized that alteration of the immunosuppressive TME may make engineered T cells restore anti-tumor effect and pave the way for improving engineered T cell function.

Transforming growth factor β (TGF-β) is one of the most important inhibitory tumor cytokines. The TGF-β impairs anti-tumor responses through negative regulation of cytotoxic cell function and promotion of T-regulatory cell maturation. In some embodiments, the engineered T cells comprise targeting molecules which neutralize TGF-β to enhance CD8+ T-cell-mediated anti-tumor immune responses.

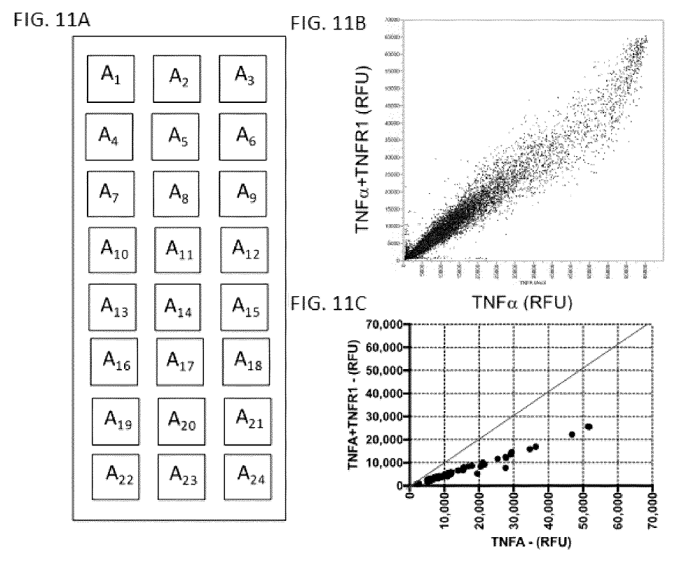

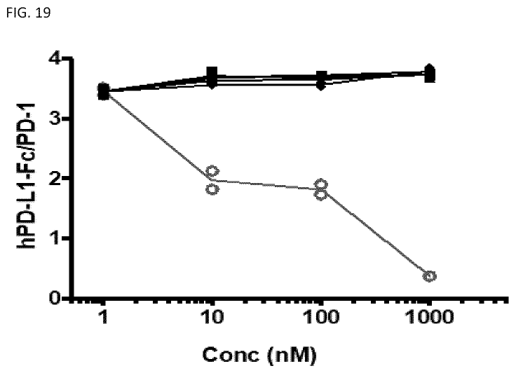

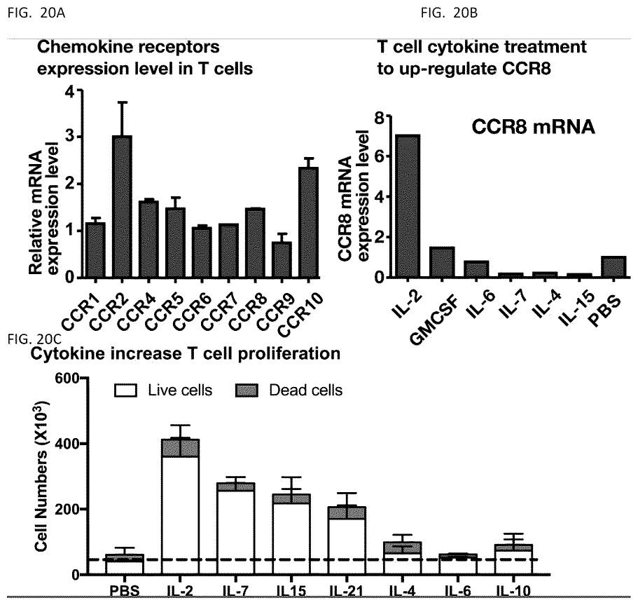

This disclosure includes methods to develop synbodies that block T cell inhibitory pathways. Synbody development for inhibitors of T cell inhibitory pathways proceeds as described herein. In addition, each target is screened alone and in complex with its receptor, similar to TNFα/TNFR1 in FIG. 11. The parallel screening enables identification of peptides that target the PPI (protein-peptide interaction) interface which is necessary to produce synbodies that inhibit the targeted PPI. Furthermore, the synbody libraries are screened by inhibition assays to identify synbodies that block the desired PPI. In some embodiments, an ELISA based assay is used to screen for PPI inhibition similar to that shown in FIG. 11. In the ELISA based assay, the receptor PD-1 is coated in the wells of a 96-well plate, and the responsive is measured as a function of aptamer, as shown in FIG. 19. The coated wells are incubated with the target protein produced as an Fc fusion, (e.g., PD-L1). Target binding is detected by an anti-Fc-HRP conjugated secondary antibody as in a standard ELISA assay. With this basic assay format, multiple candidates incubated with the target protein and then screened for inhibition to identify selected synbodies of this disclosure.

Each target protein of the PPI is commercially available and screened on high volume (e.g., 125,000) D,L-peptide microarrays. In one embodiments, the screening assay includes where TGF-B is fluorescently labeled and screened alone and in complex with its un-labeled receptor, TGFBR. In parallel, TGFBR is labeled and screened alone and in complex with un-labeled TGFB. This general format is repeated with PD-1/PD-L1 and CTLA4/B7. Peptides can be obtained from commercial peptide synthesis vendors (e.g., Polypeptides, San Diego, CA). Synbody libraries are prepared for each target as described herein. In some embodiments, synbody libraries are screened in parallel by SPR and by inhibition ELISA. In this manner, synbodies that have high affinity, K_{D}<10 nM, and inhibitory activity, IC₅₀<50 nM for each target are selected to be chemically conjugated to oligonucleotides for later immobilization to the staple strands of the DNA nanostructure that comprises a sequence which has a portion which is complementary to a portion of the oligonucleotide which is chemically conjugated to the synbodies.

In some embodiments, this disclosure provides for a method to develop aptamers that block T cell inhibitory pathways. Aptamers act as inhibitors by binding and inhibiting enzyme activities or protein-protein interactions. High aptamer binding affinity and specificity allows for effective inhibition. In some embodiments, T cell inhibitory pathways are blocked by blocking the related ligand and receptor interactions including TGFB/TGFB receptor, PD1/PDL-1, and CTLA4/B7. In some embodiments, the blocking occurs from the targeting molecule. In some embodiments, the targeting molecule is an scFv, aptamer, antibody, or fragment thereof to TGFβ/TGFβ receptor, PD1/PDL-1, and/or CTLA4/B7. This disclosure provides for a method of identifying and synthesizing distinct aptamers for a single target separately and then linking the aptamers to facilitate the coordinate between different aptamers and linker.

In some embodiments, this disclosure provides for a method to perform an in vitro assay of engineered T cell resistance to inhibitory pathways to confirm the ability of engineered T cells to penetrate the TME and kill cancer cells. In some embodiments, the engineered T cells comprise a targeting molecule which comprises an anti-TGFβ peptide. The cytotoxic specificity of the engineered T cells with or without anti-TGFB peptide in the presence of TGF-1, is measured using an LDH release assay. In some embodiments, expanded engineered T cells in cell medium supplemented with 3,000 IU/mL IL-2 is washed to remove excess cytokines. In the cytotoxity assays, 1×10⁴ engineered T cells and 5×10⁴ or 1×10⁵ 4T1 cells are added per well in a u-bottom 96-well plate culture for 4 hours, and LHD release is measured with CytoTox 96 Non-Radioactive Cytotoxicity Assay kit (Promega). The mean percentage of specific lysis of triplicate wells is calculated as follows: [(test counts spontaneous counts)/(maximum counts spontaneous counts)] 100%. In ELISA assays, 5×10⁴ T cells with engineered CAR is co-cultured with 1×10⁴ 4T1 cells or MDA-MB-231 cells for 12 hours, IFN γ, GM-CSF, IL-2, IL-10 is measured with specific primary and secondary antibodies.

This disclosure provides for methods of boosting T cell proliferation by associating a cytokine to the engineered T cell surface to establish enablement of using engineered T cells to kill cancer cells and to activate engineered T cells in the presence of a cancer cell.

Numerous cytokines profoundly affect T-cell development, differentiation, and homeostasis. IL-2, IL-7, IL-15, and IL-21 are members of a cytokine family whose heteromeric receptors share the common chain (c). Each cytokine is a T-cell growth factor and each can augment the T-cell antitumor immune response. In preferred embodiments, the selected cytokine is IL-2.

This disclosure provides a method to confirm the effects of cytokines for T cell survival and proliferation. Human T cells are isolated from peripheral blood and cultured with different cytokines. The inventors surprisingly discovered that T cell proliferation significantly increased after IL-2, IL-7, IL-15 or IL-21 treatment, as shown in FIG. 20. In addition, IL-2 also can elevate mRNA levels of chemokine receptor CCR8 (as shown in FIG. 20A, and FIG. 20B), which is the receptor of chemokine CCL1 but expression level is low without cytokine treatment. FIG. 20C depicts the cytokine increased T cell proliferation as a function of enriched culturing medium.

This disclosure provides a method for performing cytokine conjugation and linkage methods. After performing the chemical conjugation methods described herein, an in vitro assay of cytokine carrying engineered T cell proliferation and activities is performed where transduced engineered T cells are co-incubated in triplicate at 5×10⁴ cells/well with irradiated autologous EBV-LCLs at a 4:1 stimulator-to-responder ratio titrated concentrations of TGF-1 up to 20 ng/ml. After a 72-hour co-incubation period, wells are pulsed with 0.037 MBq (1 Ci)/well of [3H] thymidine (Amersham Pharmacia Biotech) for 18 hours, and the samples are harvested onto glass fiber filter paper for-scintillation counting (TriCarb 2500 TR; Packard BioScience).

### Example 2: Using Engineered T Cell Subcomponents on Mouse Model to Demonstrate Enablement of the Engineered T Cell Ensemble

This disclosure provides for methods of the in vivo analysis of therapeutic effects of engineered T cells to demonstrate enablement of using engineered T cells to kill cancer cells and to activate engineered T cells in the presence of a cancer cell.

In some embodiments, this disclosure provides for methods of evaluating antigen specific engineered T cell infiltration using iRFP-expressing tumor model. The NOD-SCID-IL2-receptor common gamma chain knockout (NSG) mouse can grow tumor-grafts from patients. After irradiation, this fluorophore is a classic model for confirming T cell adoptive transfer. For cancer models, all cell lines are first transduced with iRFP, which is a near-infrared fluorescence protein. In some embodiments, the iRFP is derived from the organism Rhodopseudomonas palustris and comprises the peptide sequence:

(SEQ ID NO: 13)
AEGSVARQPDLLTCDDEPIHIPGAIQPHGLLLALAADMTIVAGSDNLPEL
TGLAIGALIGRSAADVEDSETHNRLTIALAEPGAAVGAPITVGFTMRKDA
GFIGSWHRHDQLIFLELEPPQRDVAEPQAFERRINSAIRRLQAAETLESA
CAAAAQEVRKITGEDRVMIYRFASDESGEVIAEDRCAEVESKLGLHYPAS
TVPAQARRLYTINPVRIIPDINYRPVPVTPDLNPVTGRPIDLSFAILRSV
SPVHLEFMRNIGMHGTMSISILRGERLWGLIVCHHRTPYYVDLDGRQACE
LVAQVLAWQIGVMEE

The iRFP allows for monitoring tumor growth and antitumor activities of engineered T cells in real-time. In some embodiments, the engineered CAR comprises a luciferase domain which enables tracking adoptive transferred engineered T cell in mouse and co-localization with sup-Q injected iRFP-expressing tumor cells.

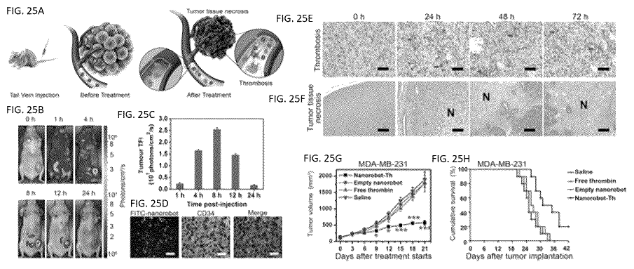

In some embodiments, the engineered T cell circulation is tracked using simultaneous bioluminescence of the iRFP and luciferase fluorescent stains. Engineered T cells that transduced with luciferase expressing lentivirus are tracked using small animal live image equipment up to seven days, as shown in FIG. 24.

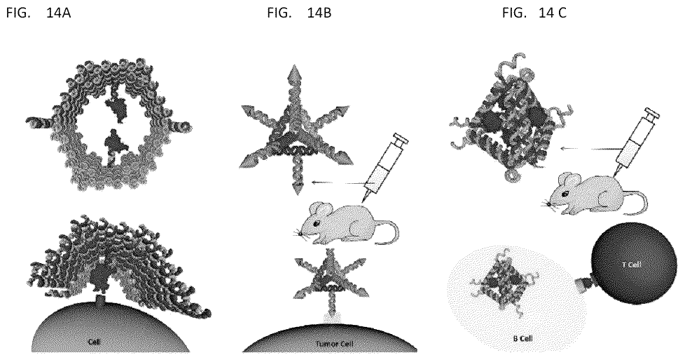

In some embodiments, this disclosure provides for method of testing the in vivo stability of DNA-nanostructures. The function of a tetrahedral DNA nanostructure (“DNA-TH”) as a delivery vehicle has been demonstrated in vivo, as shown in FIG. 14B and FIG. 24. Two near-infrared fluorescence probes are used to label DNA and streptavidin (SA). The stability and co-delivery of labeled DNA and SA protein is measured, in the form of mixtures, or assembled as complexes, to the draining lymph nodes. While the labeled DNA strands in the mixture were detectable 2 hrs post the injection, they were cleared out within 24 hrs, leaving only the labeled SA remaining at the delivery site (green). In contrast, even 96 hrs after the administration of the DNA-TH nanostructure assembled DNA-SA complex, both labeled DNA and SA were still present in the administration site and draining lymph nodes (yellow), indicating stable retention of both DNA and SA. This result confirms the structural stability of DNA nanostructures and their feasibility for in vivo applications.

In some embodiments, tumor models are monitored by transducing iRFP, a near-infrared fluorescence protein, into cancer cell lines. MDA-MB-231, which is a CD133 and mesothelin positive breast cancer cell line; BT-474 Her2+A549 which is GD2+, EGFR+, Raji CD19+ lymphoma cell to monitor tumor growth and assess anti-tumor immunity of adoptive transfer of engineered T cells with function modules in vivo. In addition, fluorophore labeled engineered CAR structure on luciferase expressing cells tracks T-cell circulation in mouse models.

This disclosure provides for a method of monitoring the tumor killing activities of the engineered T cells described herein. The MDA-MB-231-iRFP cell line is the tumor model to monitor tumor growth in vivo. To determine whether the engineered T cell target and kill tumor cells, adoptive transfer luciferase expressing engineered T cells are constructed with an IRDye800-labeled targeting molecule (e.g., CD133-synbody (“CD133 engineered T”)) or MSLN-synbody (“MSLN engineered T”), so that the engineered T cell and tumor mass volumes are followed in real time. In some embodiments, tumor tissue is collected after adoptive engineered T cell transfer and total T cell infiltration is monitored using IHC staining using an anti-CD3 antibody. Engineered T cell infiltration is monitored using an anti-SNAP-Tag™ antibody. Total tumor killing is monitored using an anti-Ki67 antibody. Target tumor killing efficiency is monitored using an anti-CD133 or anti-MSLN antibody. In some embodiments, peripheral blood is collected from engineered T cell adoptive transferred mice. T cells are isolated by Dynabeads Untouched Human T Cells Kit, using flow cytometry. Engineered T cell activation efficiency is analyzed by INF-γ or exhausting by PD-1 staining. Serum is used to quantify different cytokine release by activated T cells by ELISA. Furthermore, since MDA-MB-231 is a CD133 and MSLN double positive breast cancer cell line, engineered T cell infiltration and trafficking is measured between single targeting molecule (CD133 or MSLN) or multiple targeting molecules (CD133-MSLN) using the engineered T cells generated by the methods described herein. In some embodiments, single positive (e.g., shRNA knockdown MSLN to generate CD133 single positive or shRNA knockdown CD133 to generate MSLN single positive MDA-MB-231-iRFP cells) models are used as a tumor model to compare single or multiple targeting molecule engineered T cells.

This disclosure provides for methods of evaluating engineered T cell trafficking in a mouse model to establish enablement of using engineered T cells to kill cancer cells and to activate engineered T cells in the presence of a cancer cell. In some embodiments, an engineered T cell comprising a therapeutic agent comprising a DNA nanocage as described herein can achieve on-target tumor infarction by tunable thrombin release.

In some embodiments, DNA nanocages are used to transport molecular payloads (e.g., thrombin protein) in vivo for targeted cancer therapeutics through on-site tumor infarction. The DNA nanocage is functionalized with tumor endothelium-specific DNA aptamers displayed on its external surface and the blood coagulation protease thrombin within its inner cavity, as shown in FIG. 24. The thrombin-comprising DNA nanocage initiates tumor vessel occlusion and induces tumor necrosis, as shown in FIG. 25A. Overexpression of nucleolin on the surface of tumor endothelial cells enables nucleolin-targeting aptamers to serve as triggering molecules for mechanical opening of the DNA nanocage to release thrombin molecules which activate coagulation at the tumor sites. The DNA nanocage migrates to the tumor site as early as 4 hours after injection, as reflected by the tumor fluorescence intensity, which peaked 8 hours after injection. Minimal fluorescence was detected anywhere in the body, including the tumor site, at 24 hours after injection, as shown in FIG. 25B and FIG. 25C, demonstrating the site-specific activation of the DNA nanocage. Tumor-comprising mouse models of breast cancer are used to demonstrate that i.v. injected DNA nanocage delivers thrombin specifically to the tumor-associated vessels and induces intravascular thrombosis, resulting in inhibition of tumor growth and necrosis, as shown in FIG. 25E, FIG. 25F, FIG. 25G, and FIG. 25H. The efficacy of DNA nanocages in site-specific tumor targeting indicates the enablement of an engineered T cell comprising a targeting agent comprising a DNA nanocage for the site-specific tumor targeting to kill cancer cells and/or activate T cells in the presence of a cancer cell.

This disclosure provides for methods of testing engineered T cell recruitment in vivo with DNA-nanocage carrying chemokine or VEGF-C to establish enablement of using engineered T cells to kill cancer cells and to activate engineered T cells in the presence of a cancer cell. In some embodiments, the anti-tumor activities and immunities of the DNA nanocages are associated with the presence of the anti-nucleolin aptamer on the DNA nanocage. A combination of chemokines and cytokines is used. 4T1-OVA-iRFP breast cancer cells and OT-II transgenic mice are used as the T cell immunotherapy model. To further examine the interplay between CD4 and CD8 in coordinating antigen-specific immunity, TCR-transgenic mice (O-II) are used, wherein DO.11 CD4 T cells recognize the OVA323-339 peptide in the context of MHC-II (I-Ad of B6 background). Specifically, after 4T1-OVA-iRFP-tumors reach 4-6 mm in diameter, DNA nanocages are intra-tumorally injected. CD4 T cells isolated from OT-II transgenic mice are adoptively transferred into the aforementioned recipients at different times. Without being bound by theory, it is believed that the transferred CD4 cells are programmed in the recipients to become potent effector cells (IFNg+Foxp3-PD1-), facilitating the generation of cytotoxic CD8 cells, or to function as Treg cells, promoting the production of exhausted/immunosuppressive cells, thereby inhibiting tumor-specific CD8 cells. The antigen-specific T cell infiltration of tumor tissue is measured by the IHC staining of the useful parameters that could be used to optimize the DNA nanostructures for better and more effective anti-tumor immunity for any given tumor sample.

This disclosure provides for methods of using a combination of anti-PD-1 antibody and DNA nanocages for cancer immunotherapy to establish one embodiment of using engineered T cells to kill cancer cells and to activate engineered T cells in the presence of a cancer cell. T cell functions are related to the initiation of the PD1-PDL1 checkpoint circuit. Potent tumor regression is also applicable by applying cytokines or chemokines. Thus, the inventors have recognized that the incorporation of cytokines or chemokines into a DNA-nanocage can further enhance anti-tumor immunity by increase T cell proliferation and trafficking.

This disclosure provides for method of evaluating how multifunctional engineered T cells overcome the hostile tumor microenvironment to show that using engineered T cells kill cancer cells and to activate engineered T cells in the presence of a cancer cell. The invasion of tumor cells is a complex, multistage process, which starts with proteases including MMP degrading the basement membrane and the ECM surrounding the original tumor. In a model mouse, subcutaneous injection helps tumor cells form ECM. Since NGS is an immune deficient mouse strain, exogenous immune repressive cytokines including IL-10, IL-6 or TGFbeta are injected to sub-Q injected tumors to mimic tumor microenvironment. The tumor inhibition induced by the DNA nanocage without overt in vivo pro-inflammatory activity or toxicity is established.

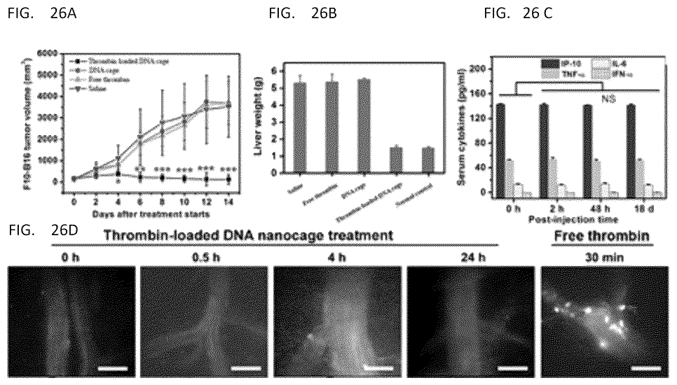

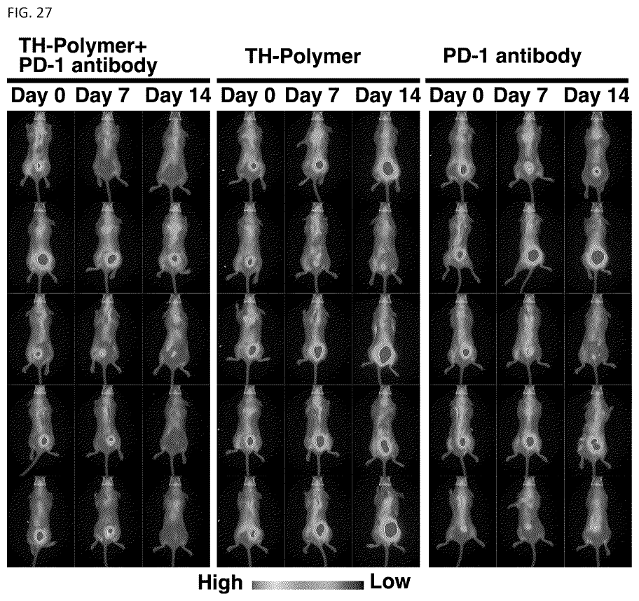

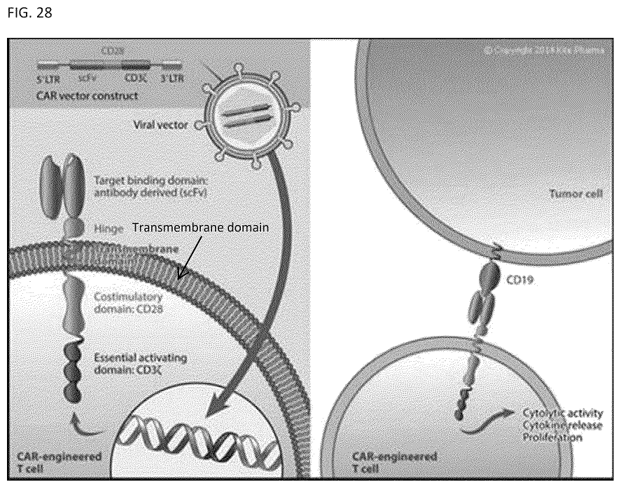

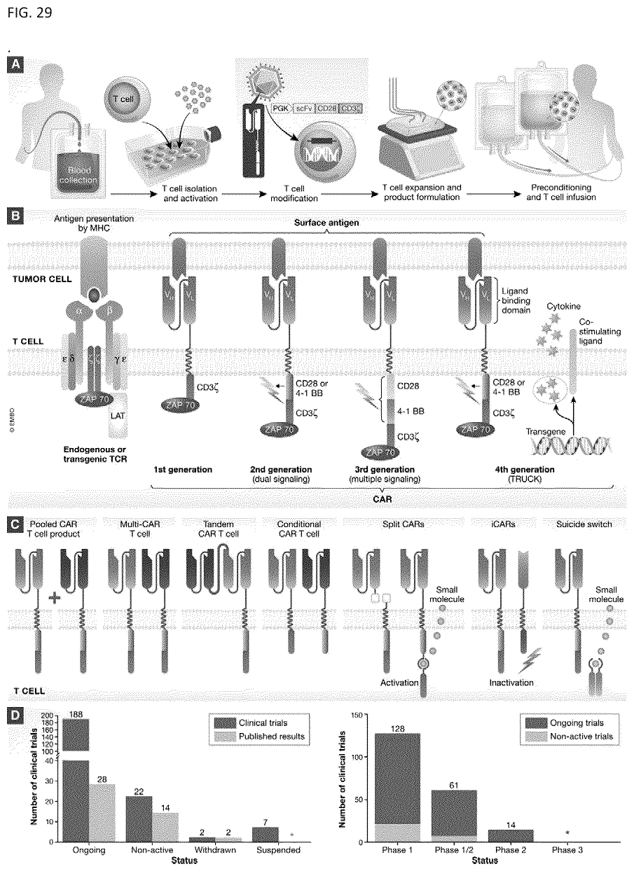

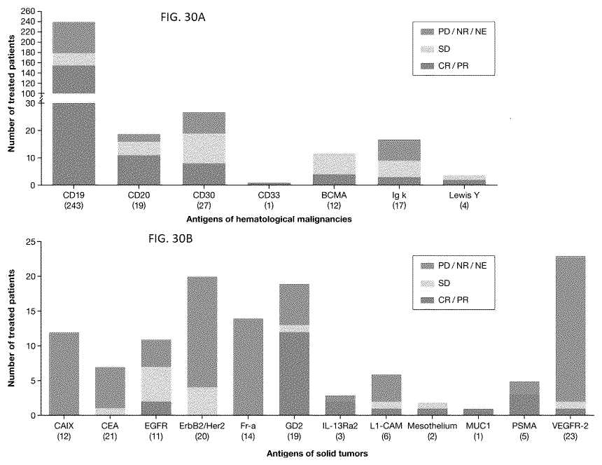

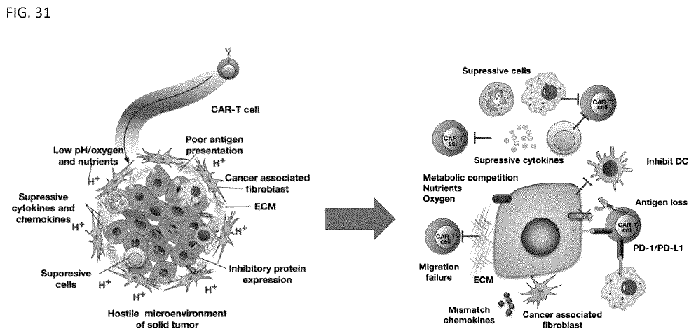

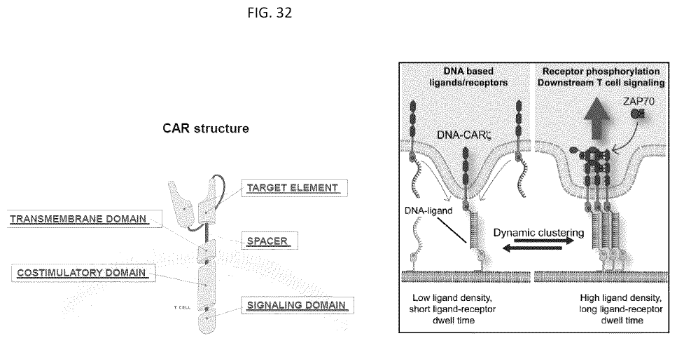

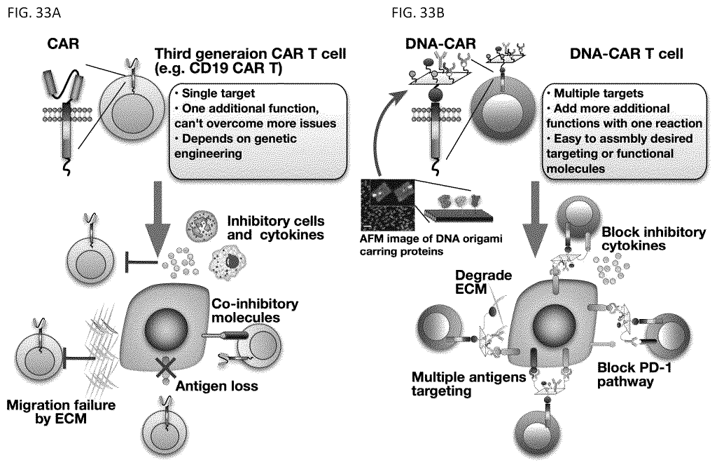

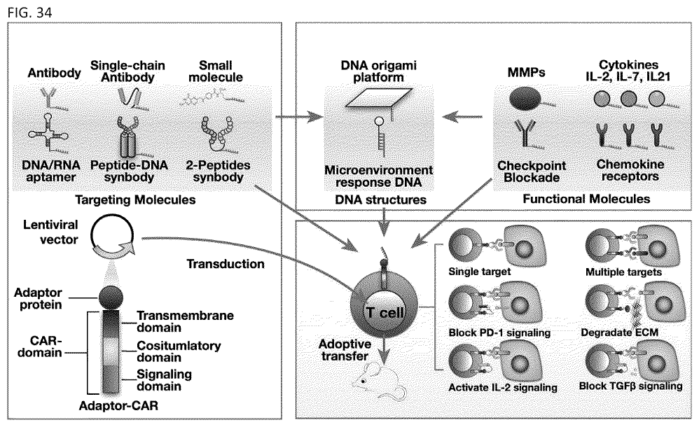

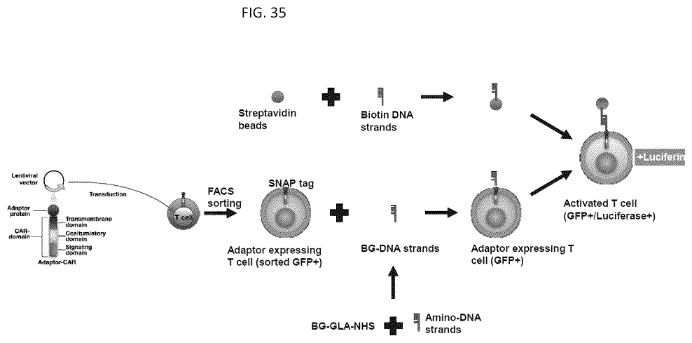

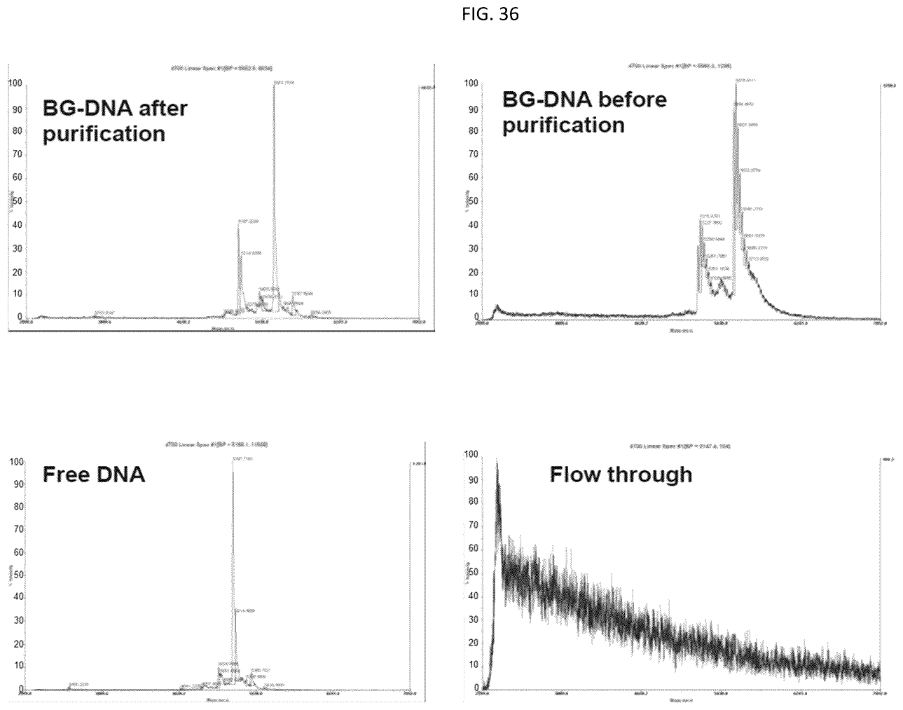

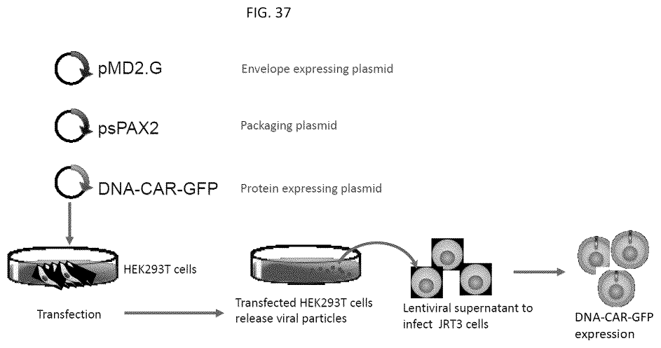

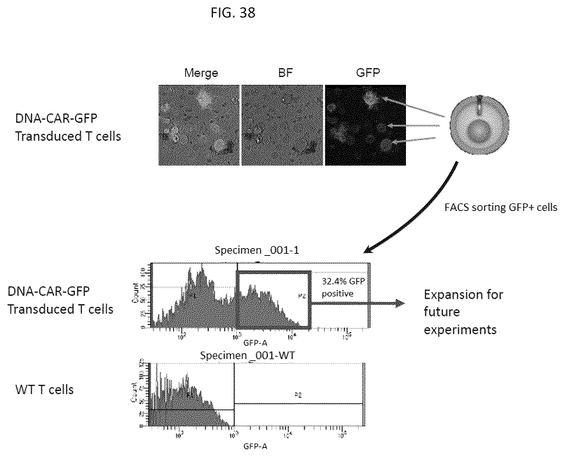

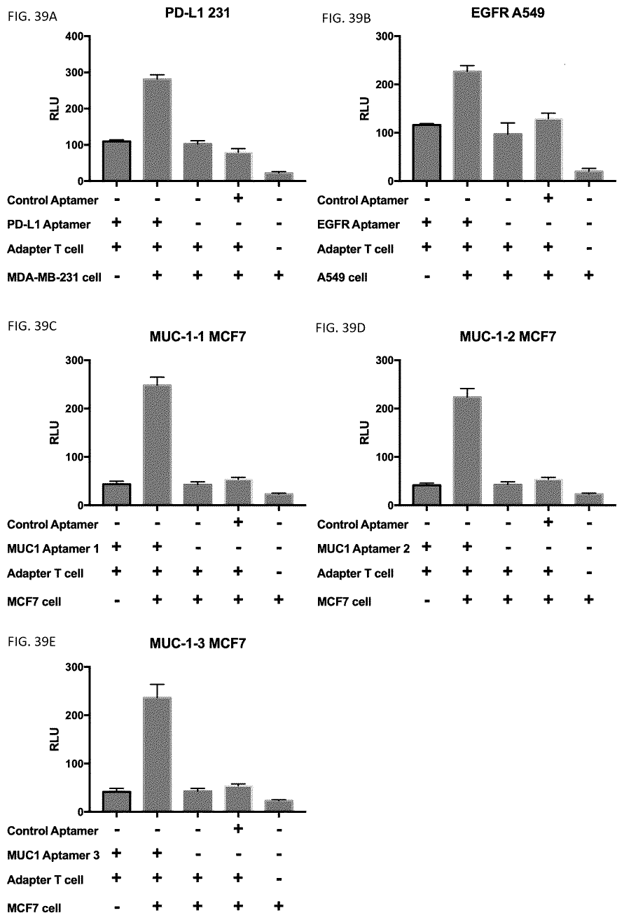

Using a syngeneic melanoma model (F10-B16), the inventors demonstrated that the transiently delivered thrombin-loaded DNA nanocage, but not free thrombin or DNA nanocage, induced significant reduction of tumor loads at both the primary inoculation site and the metastatic liver (FIG. 26A and FIG. 26B). However, the injected thrombin-containing DNA nanostructures caused no elevation of pro-inflammatory cytokines in the serum (FIG. 26C). Thrombin has been shown to initiate a coagulation cascade, leading to thrombosis formation within blood vessels. To determine whether thrombin-loaded DNA nanocages also inflict this side effect, a cerebral microthrombi formation assay was used to assess possible leakage of enwrapped thrombin from the DNA nanocage. As shown in FIG. 27D, no microthrombi were detected in MDA-MB-231-comprising mice treated with thrombin-loaded DNA-nanocages, although free thrombin did induce microthrombi formation. Thus, the DNA nanocage designed to induce tumor infarction demonstrates a good safety profile. These findings are in line with the lack of a sequence motif in the DNA nanostructures because this motif was implicated in the DNA-mediated signaling pathway for inflammation and autoimmunity

This disclosure provides for methods to evaluate engineered T cell survival in vivo to establish feasibility of using engineered T cells to kill cancer cells and to activate engineered T cells in the presence of a cancer cell.

The NSG mouse model is used to assess the in vivo antitumor effect of control and transduced engineered T cells checkpoint inhibitor modulators. 8-10-week-old male and female NSG mice are injected i.p. with either MDA-MB-231 breast cancer cells (CD133+ and MSLN+) or A549 lung cancer cells (GD2+ and EGFR+) resuspended in Matrigel (BD Biosciences). Tumors are induced by subcutaneous injection of 2×10\hat{ }6 tumor cells, and mice are treated by IV injection of T cells as indicated. For re-challenge experiments, mice are injected subcutaneously with 0.5×10\hat{ }5 cells in the flank opposite to the site of the previously rejected tumor. All experiments are randomized and blinded. Tumor growth and condition of mice are monitored every other day. For antitumor efficacy, six to eight mice per group are used. To further assess the potency of PD-L1 or CTLA4 engineered T cells, mice are treated with a mixture comprising subcutaneous MDA-MB-231 tumors with PD-L1 or CTLA4 engineered T cells. PD-L1 or CTLA4 engineered T cells induced superior antitumor immunity as compared with mice receiving engineered T cells without PD-L1 or CTLA4 peptide. PD-L1 or CTLA4 engineered T cells is either stimulated with anti-CD3 antibody, anti-CD3 plus anti-CD28 antibodies or with anti-CD3 antibody plus recombinant PD-L1 and resulting IFN-γ release is measured by ELISA. PD-L1 or CTLA4 engineered T cells are either left unstimulated or stimulated with anti-CD3 antibody or with anti-CD3 antibody plus recombinant PD-L1 and phosphorylation of AKT is measured by flow cytometry or Western blotting. Tumor samples are collected after adoptive transfer (from day 1 to day 10) to check T cell infiltration into the tumor by CD3 staining. Peripheral blood samples are collected to analyze T cell activities by flow cytometry and IFNγ, IL-2 and PD-1 staining.

This disclosure provides for methods of the characterization of immune cells in tumor draining lymph nodes (TDLNs) to demonstrate feasibility of using engineered T cells to kill cancer cells and to activate engineered T cells in the presence of a cancer cell.

The TDLN microenvironment has a significant modulatory influence on host immune responses for tumor rejection or tumor tolerance and metastasis. To determine whether DNA nanostructure module 1 can induce ICD and break tumor tolerance, the TDLN environment is examined by analyzing various cell types in the lymph nodes, including DCs (CD11c/CD80, CD86), activated T cells (CD8/CD69 and CD4/CD69), Treg or suppressive/exhausted T cells (CD4/CD8/CD25/KLRG1/PD1/Foxp1), and MDSC (CD11b/Gr1/Lyc6). Lymph nodes are removed 2 to 7 days after intra-tumoral injection to examine the presence of A20 tumor cells and characterize various types of immune cells and their functional status. Fluorescence microscopy is performed on frozen tissues or flow cytometry-based cell phenotyping of suspension cells for these analyses.

This disclosure provides for the combination of DNA-TH and anti-PD1 for elicitation of anti-tumor immunity to demonstrate feasibility of using engineered T cells to kill cancer cells and to activate engineered T cells in the presence of a cancer cell. The DNA-TH nanostructure assembled with CpG was demonstrated to show anti-tumor immunity. An iRFP expressing A20 line was created, in which iRFP is a near infrared (NIR) protein, which allows assessment of tumor growth in real-time. Here, 15 mice injected with A20-iRFP tumor cells subcutaneously in the back developed palpable and imaginable tumor lump (3-6 mm in diameter) within 8 days. Since PD-L1 is highly expressed in A20 cells, these mice were randomly grouped and given with intra-tumor injection of 1) DNA-TH-CpG followed by anti-PD1 antibodies. 2); DNA-TH-CpG and 3) anti-PD1 antibodies. As shown in FIG. 27, the combination of DNA-TH-CpG with anti-PD1 antibody resulted in a significant reduction in tumor size whereas in the other groups many mice showed enlarged tumors post the treatment. Furthermore, the mice with tumor regression observed in the combination treatment group were also resistant to the lymphoma re-challenge at a different site 30 days later, indicating their development of systemic anti-tumor immunity.

This disclosure provides for methods to evaluate the resistance to immune repression of engineered T cell with peptide inhibitor to demonstrate enablement of using engineered T cells to kill cancer cells and to activate engineered T cells in the presence of a cancer cell.

Immunodeficient NSG mice are subcutaneously inoculated with 1×10⁶ MDA-MB-231 cells. When tumors are approximately 200 m³, mice are given 107 CAR-expressing engineered T cells via intravenous administration. Tumor volume is monitored by caliper measurements twice weekly, and at different time points, tumor-comprising mice are sacrificed for mechanistic studies using immunoblotting and/or flow cytometry. All experiments had a minimum of 5 mice per group and were performed at least three times. The phosphorylation status of various proximal T-cell signaling entities was assessed by immunoblotting (pERK, pAkt, pLck) using engineered T cells and plate-bound CD3/CD28 antibodies. Engineered T cells were also exposed to mesothelin-coated beads as described and harvested after 20 minutes. For ex vivo T-cell analysis, tumors were harvested from mice, microdissected, digested, and used in ex vivo tumor assays as described previously.

To confirm engineered T cells can elevate endogenous immune response, in vivo CTL proliferation of leukocytes is measured by 5-bromo-2-deoxyuridine (BrdU) incorporation according to the manufacturer's specifications. BrdU is added to the drinking water and provided throughout the duration of the study. Four mice per group is killed, and spleens and livers is harvested 20 days after engineered T cell injection.

To evaluate whether engineered T cells with overcome Treg repression in vivo, Tregs are isolated from spleens of naive wild-type (WT) mice by MACs column selection to more than 90% purity. CD8 T cells from WT mice are isolated by MACs column selection and transduced with and labeled with 1M carboxyfluorescein succinimidyl ester (CFSE). T and NK cell-depleted splenocytes from WT mice are used as APCs. A total of 5×10\hat{ }4 CFSE-labeled CD8 T responder cells is stimulated with 1.5×10⁵ APCs in RPMI-c and 1 g/mL purified anti-CD3. Where indicated, 1×10⁵ Tregs from WT mice and/or anti-TGFB or control IgG at 0.5 g/mL is added to the culture. Four days later, cells are harvested and proliferation is determined by CFSE dilution. Cell supernatant is harvested for IFN-γ levels as determined by IFN-γ ELISA.

To assess in vivo suppression, 10\hat{ }6 Tregs isolated from breast cancer-comprising mice (25 days after MDA-MB-231 injection) is transferred into WT mice that had been injected with 1×10⁴ engineered T cells 7 days before the adoptive transfer. Anti-TGFβ or control rat IgG (200 g/dose) is given every other day from day 3 to day 14 after engineered T cell injection. Thirteen days after the engineered T cell injection, spleens and livers are harvested. Leukocytes from livers are isolated with discontinuous Percoll gradient centrifugation. Cells are stimulated for 5 hours in vitro with CD3 (1 μg/mL) and CD28 (1 μg/mL), and labeled with CD8-FITC, IFN-PerCP, and CD45.1-allophycocyanin, and 100 000 live events is gated to determine the percentage of IFN-producing CD8 CTLs.

In vivo CTL proliferation of leukocytes is measured by 5-bromo-2-deoxyuridine (BrdU) incorporation according to the manufacturer's specifications. BrdU is added to the drinking water and provided throughout the duration of the study. Four mice per group is killed, and spleens and livers are harvested 20 days after engineered T cell injection.

This disclosure provided for a method for an in vivo ECM degrading T cell invasion assay to demonstrate feasibility of using engineered T cells to kill cancer cells and to activate engineered T cells in the presence of a cancer cell.

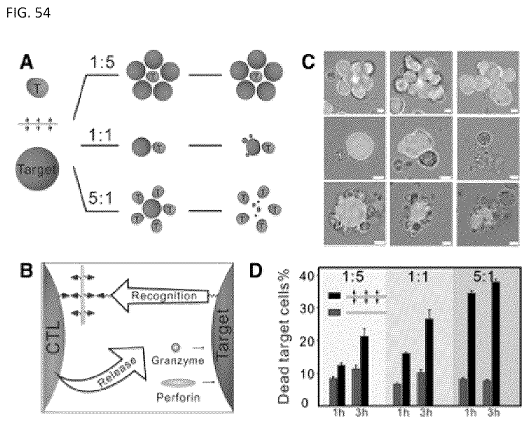

In some embodiments, this disclosure provides for a method of using DNA origami assisted lymphocytes to kill tumor cells. CD8+ cytotoxic T lymphocyte (CTL) cells were first activated by CD3 and CD28 signal pathways resulting in perforin granule release from the CTL. The perforin/granzyme cell pathway requires direct contact between target cancer cells (“T”) and effector cells (“E”), as shown in FIG. 54. CTL without an antigen in target cell (L1210) surfaces therefore cannot recognize target cells. However, when assisted by a DNA origami nanostructure comprising CD8+, different mixtures of cell patterns were observed in a dose-dependent manner (FIG. 54), which demonstrated that the DNA origami facilitated interactions between effector and target cells. Thus, the DNA origami nanostructure can interact with a T cell surface to kill tumor cells. In some embodiments, the DNA origami nanostructure is that described in U.S. Pat. No. 8,440,811, herein incorporated by reference.

### Example 3: Adaptor T Cell (SNAP_GS4_TM_41BB_CD3z_GFP) Production

### A. Preparation of Lentivirus Supernatant.

Slited 293T cells cultured on 10% FCS/DMEM/PS medium were trypsinized 10-15 seconds overnight. Next, 9 to 10 million overnight culture of 293 T cells were plated onto each 10-cm poly-lysine coated plate using 10 mL of 10% FCS/DMEM medium. The cells were allowed to let sit in a lamellar hood for 10 to 20 minutes to allow the cells to evenly distribute on the plate surface. The cells were then carefully transferred to the CO₂ incubator. The cells were allowed to grow for 4 to 6 hours to 80% confluence. The growth medium was removed by aspiration. The cells were then quickly overlayed with pre-warmed 5 mL of OPTI-MEM medium and quickly put back into the incubator.

DNA/Lipofectamine 2000 complexes were prepared from the following: 1.5 mL OPTI medium, pLenti-EF1a_SNAP_GS4_TM_41BB_CD3z_GFP 13 μg, pMD2.G 6 μg, and psPAX2 9 μg.

In a separate tube was added 60 μl of Lipofectamine was added in 1.5 mL OPTI medium. The two tubes were combined and mixed and incubated in a hood for 20 minutes. Next was added the DNA/Lipofectamine mixture to the Cells dropwise. The plate was gently swirled to distribute the mixture throughout the plate. After 6 hours of transfection, the DNA mixture was removed and add 5 mL of pre-warmed 5% FCS/DMEM/5 mM sodium butyrate viral production medium was added to collect the virus particles. On the second day, another 5 mL of medium was added. A total of 10 to 12 mL of medium was added per plate. The supernatant was harvested after 48 hours of culturing. Care was taken to ensure that the medium did not turn yellow because the half-life of the virus is less than 10 minutes at pH 6.0 or 8.0

The supernatant was centrifuged at 1500 rpm for 5 minutes to remove debris and filter the supernatant through a 0.45 μM filter. The supernatant was centrifuged at 25,000 rpm for 2 hr at 4° C. The supernatant was then gently aspirated. The tubes were inverted on a paper towel for 4-5 minutes to remove most of the remaining liquid that inhibits infection efficiency and T cell proliferation. Next, was added 100 μL of 1% FCS/PBS (Ca²⁺/Mg²⁺ free PBS) on the tube and the tube was then sealed and vortexed at the lowest speed on a table-top vortexer for at least 2 hr at 4° C. The resuspended virus was centrifuged at 5000×g for 5 min at 4b° C. to remove debris. Next, the supernatant was flash frozen on dry ice and stored at −80° C.

### B. Infection of Engineered T Cells.

One day before infection, engineered Jurkat T cells were seeded at a concentration of 1×10⁶/mL. Virus was added in 0.5 mL of 5% FCS/RPMI medium/(8 μg/mL polybrene) to Jurkat T cells in a 24-well plate and centrifuged for 1 hr at 1800×g at room temperature. After at least 4 to 5 hours of incubation in the incubator, 2 mL of T cell medium was added. The infection was checked every 24 hours, with repeated infections as necessary until infection was confirmed.

### Example 4: NFAT-Luc Jurkat Activation Assay

This protocol was used for end-point readings using a luminometer with an injector. This protocol can be adapted for use with kinetic measurements or a luminometer with a manual set-up.

Cells were centrifuged at 1000-1500 rpm (RC 200-300×g) for 5 min. Supernatant was removed and the Jurkat-Lucia NFAT cells at 2×10⁶ cells/mL were resuspended in fresh, pre-warmed growth medium. Next, was added 20 μL of sample/well including PMA (50 ng/mL) with ionomycin (3 μg/mL) or concanavalin A (10 μg/mL) as positive control and endotoxin free water as negative control (use new tips between). Next, was added 180 μL cell suspension (˜400,000 cells)/well of 96 well plate. Cell suspension was in RPMI or IMDM. Next, the plate was incubated at 37° C. in CO₂ incubator for 18-24 hours. The luminometer was set with the following parameters: 50 μL injection, end-point measurement w/4 second start time, and 0.1 second reading time. Samples were pipetted (10-20 μL/well) into 96 well white (opaque) OR black plate, OR a luminometer tube.

### Example 5: BG and DNA Oligonucleotide Conjugation

The DNA oligonucleotide was dissolved in HEPES buffer to prepare 1 mM stock solution.

Fresh 50 mM TCEP was prepared in 10 mM pH 7.4 sodium HEPES buffer and adjusted pH to ˜7.0 using NaOH. Next, 10-fold pH 7.0 TCEP (50 mM stock) 10 μL was added into 1 mM DNA stock 50 μL, shaken at room temperature for 2-3 hours, and store in a 4° C. refrigerator. Next was added 5 fold BG-maleimide (dissolved in DMF, 50 mM) 5 μL into deprotected thiol-modified DNA (in HEPES buffer, 1 mM) 50 μL. Then, a defined volume of PBS buffer was added to reach final DNA concentration of 200 μM. The reaction was allowed to proceed overnight at room temperature. The reaction was washed with PBS buffer (using 3 kD Amicon centrifugal dialysis filters) at 13000 rpm for 20 min for 3 times. The products were quantified by adsorption of 260 nm. BG-DNA was stored in −20° C. freezer.

### Example 6: BG-DNA and Jurkat T Cell Conjugation

Adaptor T cells expressing the engineered CAR with a SNAP-Tag™ adaptor protein were washed two times with PBS buffer, then resuspended 2 million cells in 50 μL DPBS buffer of defined concentration of BG-DNA. The reaction was allowed to proceed for 30 min at room temp. The reacted cells were washed 2 times with DPBS buffer, then resuspended in 1 μM BG-T 20 (or A20) 50 μL. The reaction was allowed to proceed for 30 min, then washed 2 times again. The conjugated T cells were imaged in confocal microscope or flow cytometry.

### Example 7: Conjugating the Targeting Molecule, Anti-PD-L1 Antibody (“aPD-L1”) with a DNA Oligonucleotide

### Thiol-Modified DNA Oligo Deprotection (TCEP Cleavage, Purification and Storage)

DNA oligonucleotide was dissolved in water to prepare a 10 mM stock. Next, fresh buffer was prepared: 50 mM TCEP in 10 mM pH 7.4 sodium HEPES buffer and adjusted pH to ˜7.0 using NaOH. Next, 250 UM, 2 mL DNA deprotection mix was prepared from a solution of 10 mM DNA stock 50 μL, 20-fold excess of pH 7.0 TCEP (50 mM stock) 200 μL was added. Next, 10 mM sodium HEPES buffer was added to reach the final volume and shook at room temperature for 2-3 hours. The mixtures was then washed with water using a centrifugal dialysis membrane (3 kD Ultracel 4 mL Amicon) at 4000 rpm for 45 min for 3 times (at a temperature of 4˜10° C.). The deprotected thiol-DNA oligos were able to be stored in −20° C. freezer for up to one month.

### II. Protein Preparation and Protein-DNA Conjugation

The SH-T20 was reacted with the crosslinker SPDP (ThermoFisher). A solution of SPDP (50 mM in DMSO), 10 μL, was added into 100 μL 1 mM DNA. The reaction was performed 37° C. for 30 min

Excess SPDP crosslinker was removed by centrifuge 130000 rpm 20 min 3 times with 3 k filter tubes, as described above. The antibody solution was prepared by in pH 7.4, 10 mM sodium PBS buffer at a concentration ˜50 μM for storage. The NHS-DNA was mixed with aPD-L1 at a ratio of 50:1, and the reaction allowed to proceed overnight at room temperature. The excess DNA oligonucleotides were removed by centrifugation 3000×g 10 min 3 times with a 100 KDa filtration dialysis tube (Amicon).

### Example 8: Test Killing Efficiency of Engineered T Cell to Target Cells

Engineered T cells were conjugated with BG-A20/BG-T20 as described above before. The reaction solution was centrifuged at 1500 rpm 2 min 2 times, the pellet isolated, and the supernatant removed by pipette to remove unreacted DNA. Next, complementary DNA (T20 or A20) covalently conjugated to the modified antibody was added. In some embodiments, aptamers are used instead of the antibody. The complex was hybridized at room temp for 30 min. The engineered T cells were then centrifuged at 1500 rpm 2 min 2 times, the pellet isolated, and the supernatant removed by pipette to remove unreacted targeting molecules. Next, target cancer cells were conjugated with the engineered T cells at a ratio of 1:5 in RPMI 1640 medium (serum inactivated). The complex was incubated in a cell incubator with 5% CO₂ for 24 h. The luciferase level was then monitored to test the engineered T cell activation level in the presence of the cancer cell.

### Example 9: Lentiviral Transfer of Plasmid Encoding an Engineered CAR

All reference to “Jurkat cells” indicate Jurkat-Lucia™ NFAT cells (Invivogen, San Diego, CA).

As shown in FIG. 42A-C, lentiviral transfer plasmid encoding one embodiment of an engineered CAR was achieved using an expression cassette comprising long terminal repeat (LTR), human elongation factor 1 alpha promoter (EF1a), human CD8-leader sequence (L), benzylguanine binding protein SNAP-Tag™ (SNAP), 4 repeats of the peptide linker GGGS-GGGS-GGGS-GGGS (4XGS) (SEQ ID NO: 14), human CD8 transmembrane domain (TM), human 4-1BB, human CD3ζ and GFP. As shown in FIG. 42B, the procedure of transferring lentiviral vector into Jurkat T cells was as follows: NFAT-Luc Reporter T lymphocytes were used as a model to study T cell activation. Jurkat-Lucia™ NFAT cells were derived from the human T lymphocyte-based Jurkat cell line by stable integration of an NFAT-inducible Lucia reporter construct. The Lucia gene, which encodes a secreted coelenterazine-utilizing luciferase, was driven by an ISG54 minimal promoter fused to six copies of the NFAT consensus transcriptional response element. Jurkat-Lucia™ NFAT cells were used for the study of NFAT activation following stimuli treatments by monitoring Lucia luciferase activity. Levels of Lucia luciferase were readily measurable in the cell culture supernatant when using QUANTI-Luc™, a Lucia luciferase detection reagent. As shown in FIG. 42C, flow cytometry results show that GFP positive Jurkat cells transduced with lentivirus encoding engineered CAR (FIG. 42C) were isolated and sorted by flow cytometry.

### Example 10: Conjugating Functional Agents to Engineered T Cells Comprising an Engineered CAR Domain

Engineered T cells comprising an engineered CAR which further comprises an adaptor protein were able to conjugate a protein tag which was covalently conjugated to a first oligonucleotide, and then hybridize a second oligonucleotide comprising a functional agent to the first oligonucleotide. In some embodiments, the functional agent is a targeting agent. In some embodiments, the functional agent is a fluorophore.

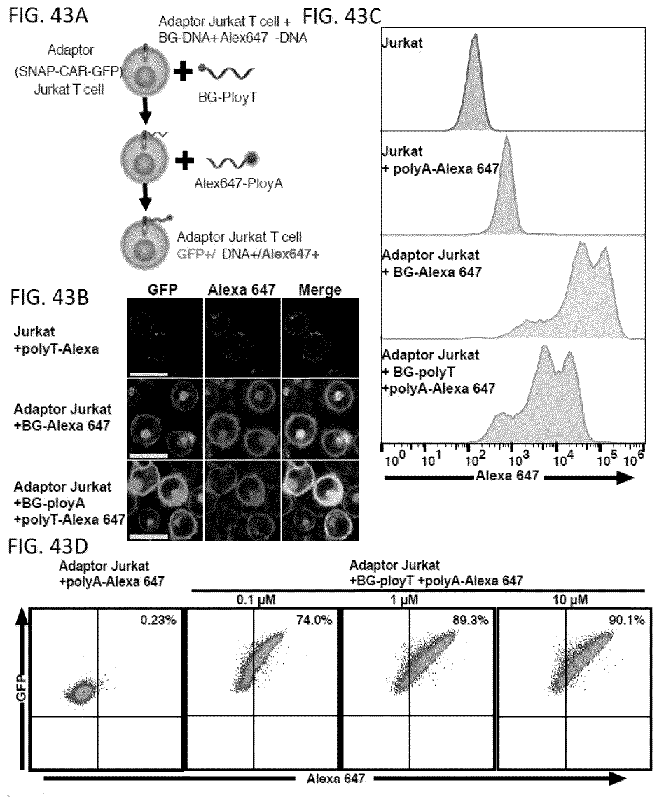

As shown in FIG. 43A-D, adaptor Jurkat T cells conjugate BG (benzoguanine)-modified ssDNA (polyT) and hybridize with ssDNA (Alexa647-labelled polyA). The first oligonucleotide comprises the sequence of a polyT strand (15 nt). The second oligonucleotide comprises the sequence of a polyA strand (15 nt). The Alexa647-labelled polyA was obtained from commercial vendors (IDT). The BG-modified oligo was prepared as described above. As shown in FIG. 43B, confocal imaging of adaptor Jurkat T cells were linked with a functional molecule (e.g., Alexa647 fluorophore) through immobilization of a DNA strands (e.g., a first oligonucleotide and a second oligonucleotide). The GFP signal represents the starting material, the Alexa647 signal represents the location of the product. The merged image identifies that the fluorophore-labelled oligonucleotide selectively hybridizes to the first oligonucleotide on the adaptor T cells (engineered T cells) which were conjugated with BG-polyT. Alexa 647 and GFP double positive cells indicate DNA strands have successfully conjugated on Jurkat cells. BG-Alexa 647 was used as a positive control.

FIG. 43C shows the fluorescence intensity of Alexa 647 on Adaptor Jurkat T cells (engineered T cells). Alexa647-polyA intensity of Jurkat T cells were analyzed by flow cytometry. Alexa647 intensity of Jurkat with adaptor was higher than Jurkat T cells without adaptor structures, indicating that the second oligonucleotide comprising a functional agent was immobilized to the engineered T cell. As shown in FIG. 43D, the ratio of Alexa 647 positive Adaptor Jurkat T cell to that of GFP is displayed for control (adaptor Jurkat+polyT-Alexa647) cells to the hybridized cells (Adaptor Jurkat+BG-polyA+poltT-Alexa). The ratio increased when higher concentration BG-polyA was add to conjugate DNA, demonstrating that the functional agent was associated to the adaptor T cells (engineered T cells) via hybridization of the first oligonucleotide to the second oligonucleotide.

### Example 11: Engineered T Cells are Activated in the Presence of a Target

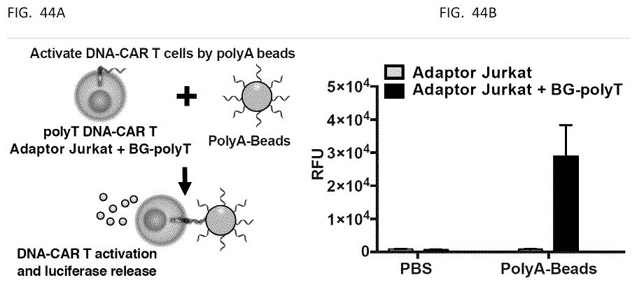

As shown in (new FIG. 44A-B), the engineered T cells were activated upon hybridization to a target. The model target was a polyA-coated agarose bead (15 microns in diameter). The polyA-coated agarose beads were prepared by standard methods to create a model cancer cell target. NHS-activated agarose beads conjugated with NH2-polyA (“polyA beads”) (polyA sequence was 15 nt long) and engineered T cells were incubated for 12 hours. T cell activation levels were monitored by relative luminescence units (RLU) (also referred to herein as relative fluorescence units (RFU)) of released luciferase in the cell medium, as per the manufacturer's instructions for measuring RLU. As shown in FIG. 44B, engineered T cells were activated by DNA strands on agarose beads surface only when the engineered T cell comprises a first oligonucleotide which was connected to the adaptor protein. In addition, T cell activation did not occur in PBS control solution, indicating that the engineered T cells were activated in the presence of a model target for a cancer cell.

### Example 12: Engineered T Cells were Activated in the Presence of a Target Cancer Cell

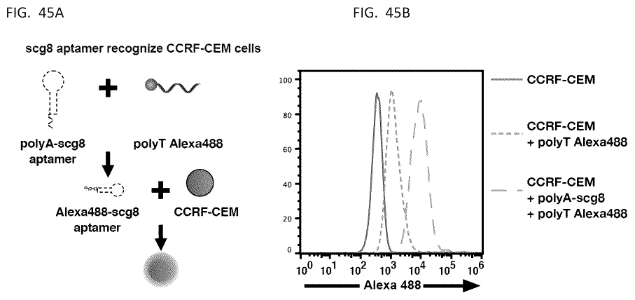

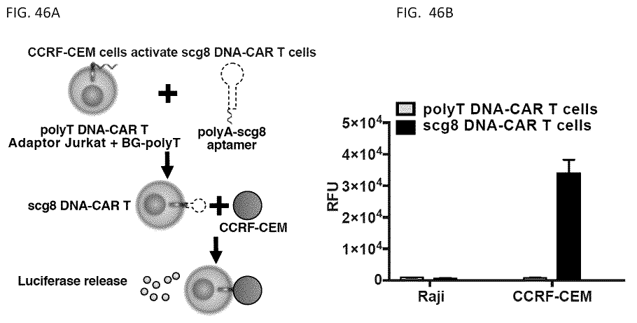

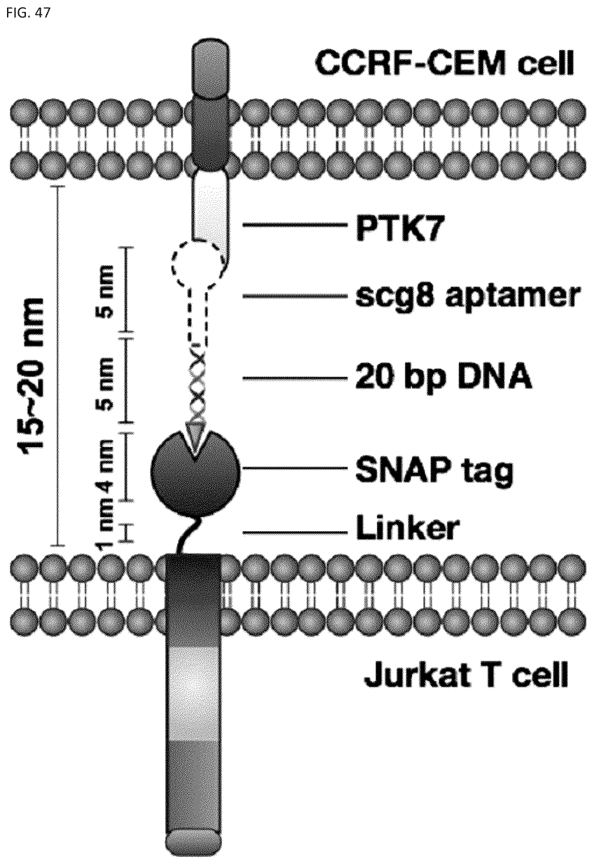

As shown in FIG. 45A and FIG. 45B, engineered T cells comprising cancer cell-specific aptamers were activated in the presence of a cancer cell. The engineered T cells comprising ssDNA of polyT were prepared as described above. A polyA-scg8 aptamer was prepared using the methods described herein. The target cancer cell was CCRF-CEM cells. CCRF-CEM is a T lymphoblastoid line obtained from the peripheral blood of a 4 year old Caucasian female with acute lymphoblastoid leukemia which targets protein tyrosine kinase 7 (PTK7) on CCRF-CEM cell membrane. The construct for the engineered T cell is depicted in FIG. 47, which depicts the relative spacing differences between the linker domain, the SNAP-Tag™ protein, the first and second oligonucleotides hybridized, the aptamer, and the total cell-cell separation. The distances were approximately 1 nm for GS-linker, 4 nm for SNAP-Tag™, 5 nm for 20 double-stranded DNA, and 5 nm for scg8 aptamer, with the total cell-cell distance about 15 nm to about 20 nm separation. The inventors have recognized and designed constructs for, in some embodiments, the GS spacer and/or the first and second oligonucleotide lengths can be modulated to increase or decrease the total cell-cell distance to enhance the potency of the engineered T cell in the presence of a cancer cell.

PolyA-scg8 aptamers hybridized with polyT-Adaptor488 to produce Alexa488-scg8. The binding efficacy of scg8 was analyzed by flow cytometry. As shown in FIG. 56B, the fluorescence intensity of Alexa488-scg8 labeled CCRF-CEM demonstrates that Alexa488-scg8 recognized the target cancer cell CCRF-CEM. Thus, engineered T cells comprising targeting molecules (e.g., the scg8 aptamer) bind to target cancer cells.

As shown in FIG. 46A-B, CCRF-CEM cancer cells activated engineered T cells. As shown in FIG. 46A, adaptor Jurkat T cell (engineered T cell) conjugated with BG-polyT generated polyT engineered T cells. CCRF-CEM cells and scg8 DNA-CAR T cells were incubated for 12 hours, after which T cell activation levels as measured by luciferase activity were measured by relative luminescence units (RLU). As shown in FIG. 46B, engineered T cells were activated by cancer cells, whereas the engineered T cells were not activated in the presence of non-cancerous cells (“Raji”).

### Example 13: Engineered T Cells are Activated in the Presence of a Target Tumor Cancer Cell

As shown in FIG. 39, engineered T cells comprising targeting molecules which also comprised a tumor cell-specific aptamer were activated in the presence of said tumor cell. The cancer tumor cells were MDA-MB-231, which was a Programmed death-ligand 1 (PD-L1) positive human breast cancer cell line; A549, which was an epidermal growth factor receptor (EGFR) positive lung cancer cell line; and MCF7, which was a Mucin 1 (MUC1) positive breast cancer cell line.

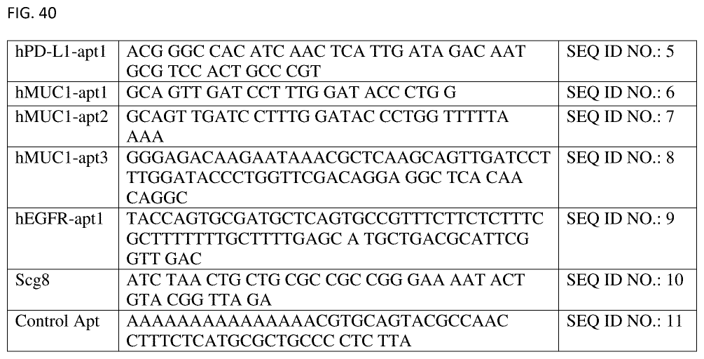

Engineered T cells comprising aptamers were prepared as described herein. The aptamer sequences used were those described herein, including those in Table 1 and FIG. 40. MDA-MB-231 breast cells and anti-PD-L1 comprising engineered T cells were incubated for 12 hours, after which T cell activation levels as measured by luciferase activity were measured by relative luminescence units (RLU). The “+” indicates the species was present, and “−” indicates the species was absent for each column on the bar graphs. The second column in FIG. 39A shows that anti-PD-L1 engineered T cells were activated by MDA-MB-231 cells. Only low levels of luciferase release were observed in T cell alone, tumor cell alone, T cells without aptamer or with T cells with a control aptamer group. The results demonstrate that engineered T cells were activated in the presence of a solid tumor cell.

A549 lung cancer cells and anti-EGFR engineered T cells were incubated for 12 hours after which T cell activation levels as measured by luciferase activity were measured by relative luminescence units (RLU). As shown in FIG. 39B, the second column shows that anti-EGFR engineered T cells were activated by A549 cells. Low levels of luciferase release were observed in T cell alone, tumor cell alone, without aptamer or with control aptamer group. The results demonstrate that engineered T cells were activated in the presence of a solid tumor cell.

As shown in their respective figures FIG. 39C, FIG. 39D, and FIG. 39E, the second columns show that three different anti-MUC1 engineered T cells were activated by MCF7 cells. Low levels of luciferase release were observed in T cell alone, tumor cell alone, without aptamer or with control aptamer group. The results demonstrate that engineered T cells prepared by the methods described herein were activated in the presence of a variety of different solid tumor cell types.

Engineered T cell activation further results in tumor cell death in the presence of said tumor cell.

### Example 14. Engineered T Cells Comprising a scFv with Different Linker Morphologies are Activated Under a Selected Stimulus

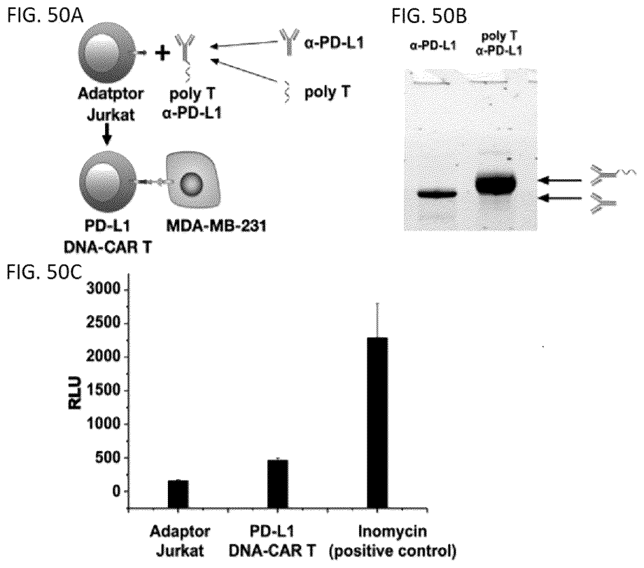

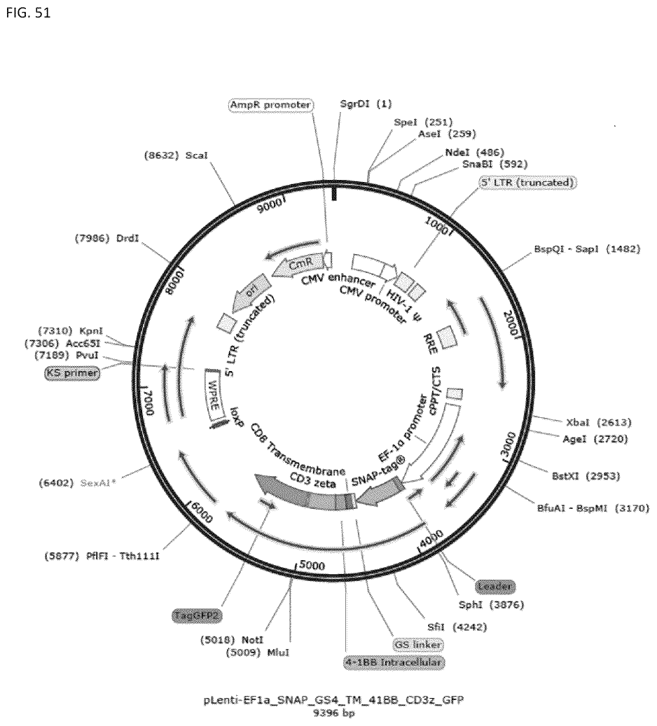

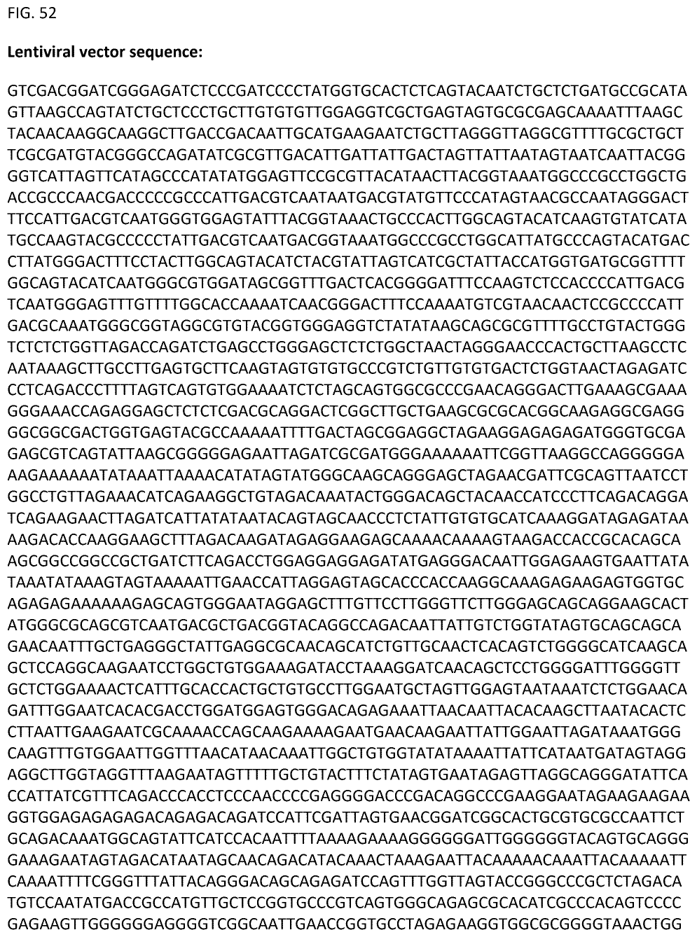

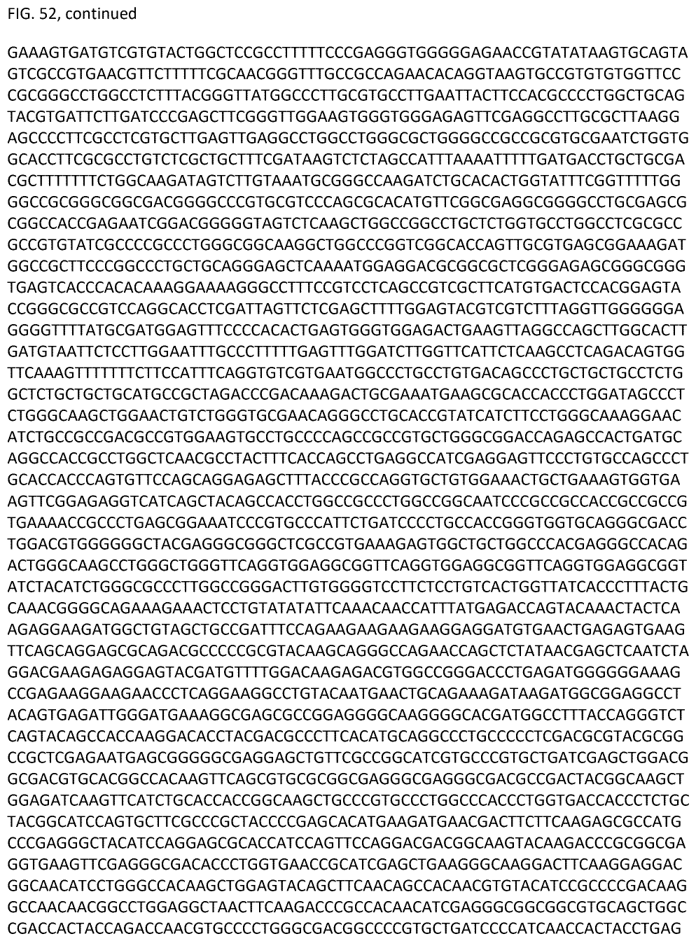

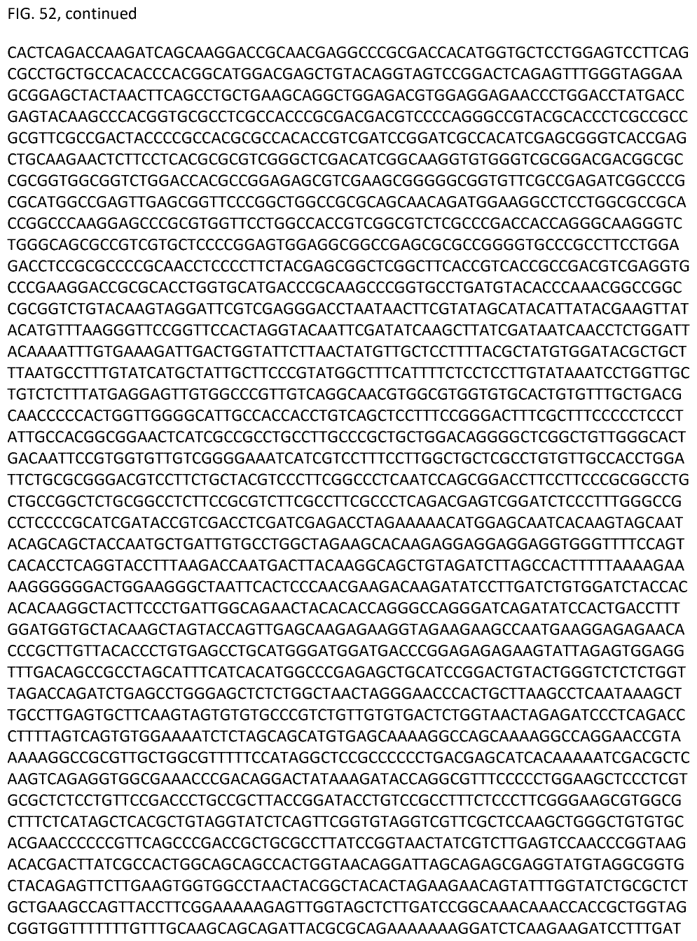

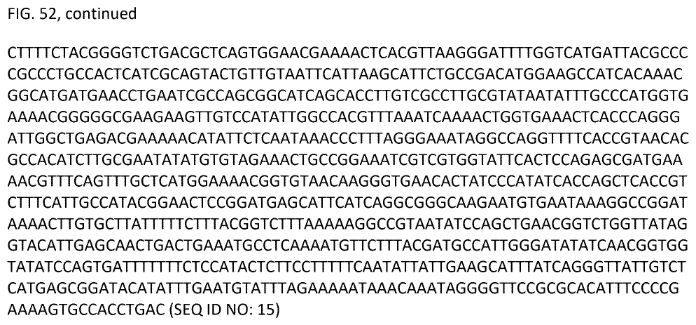

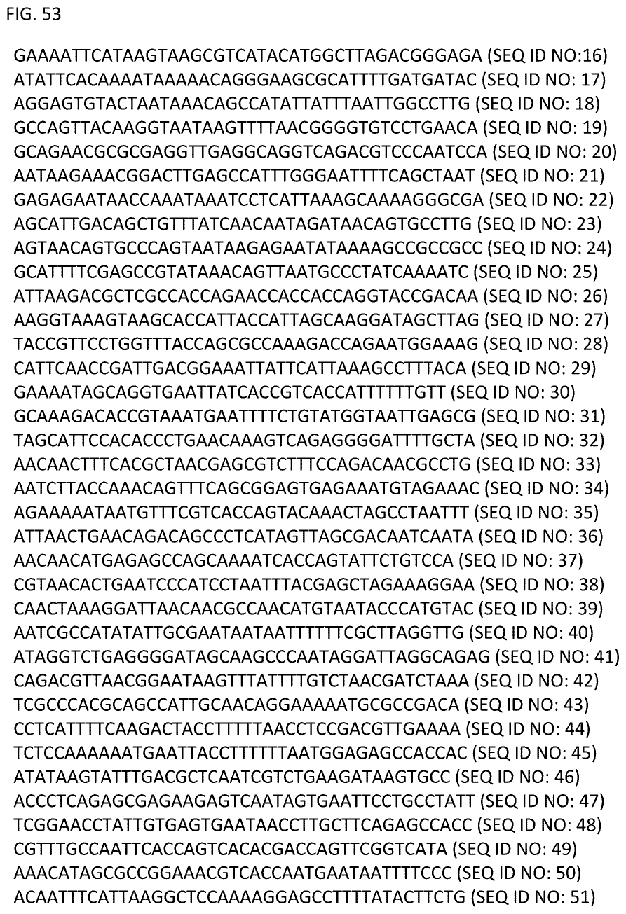

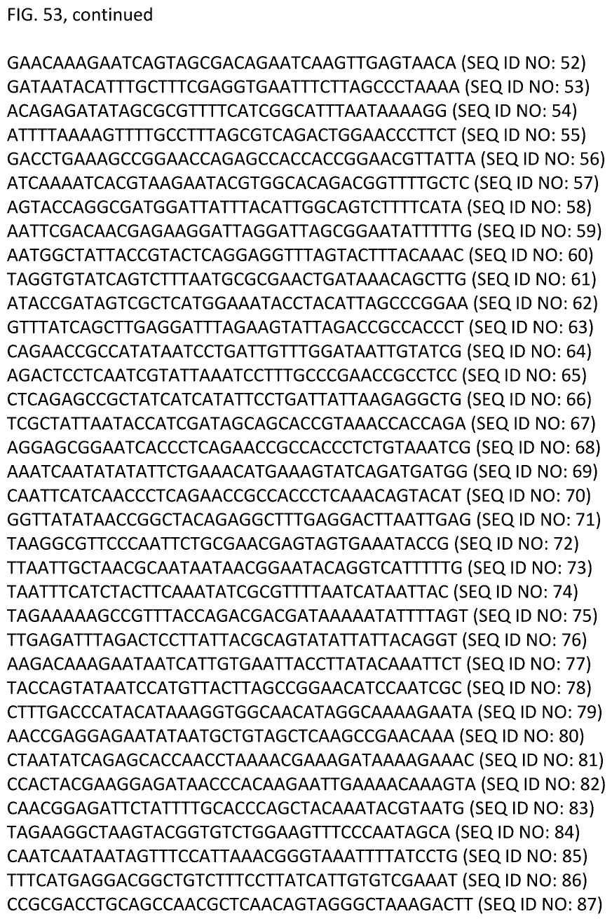

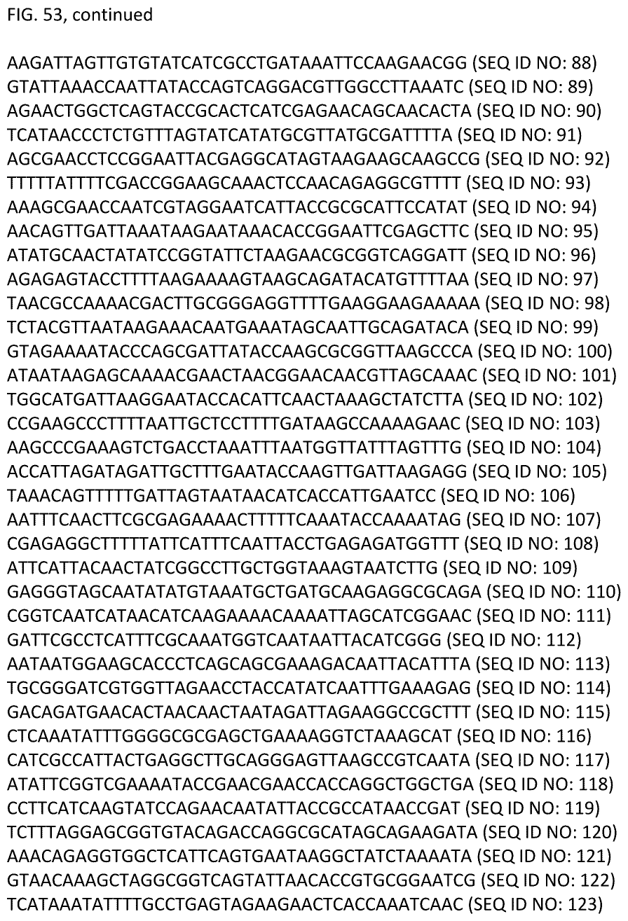

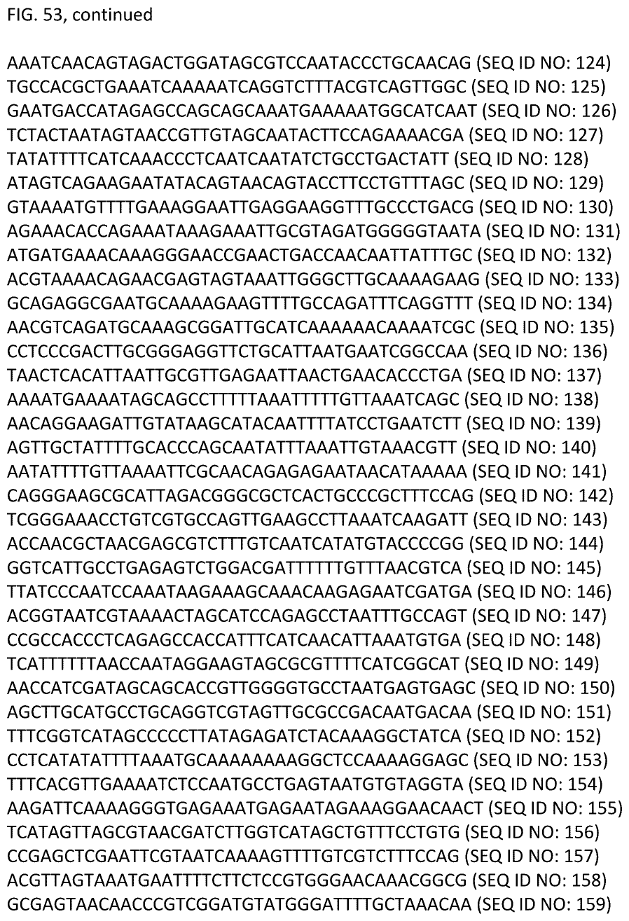

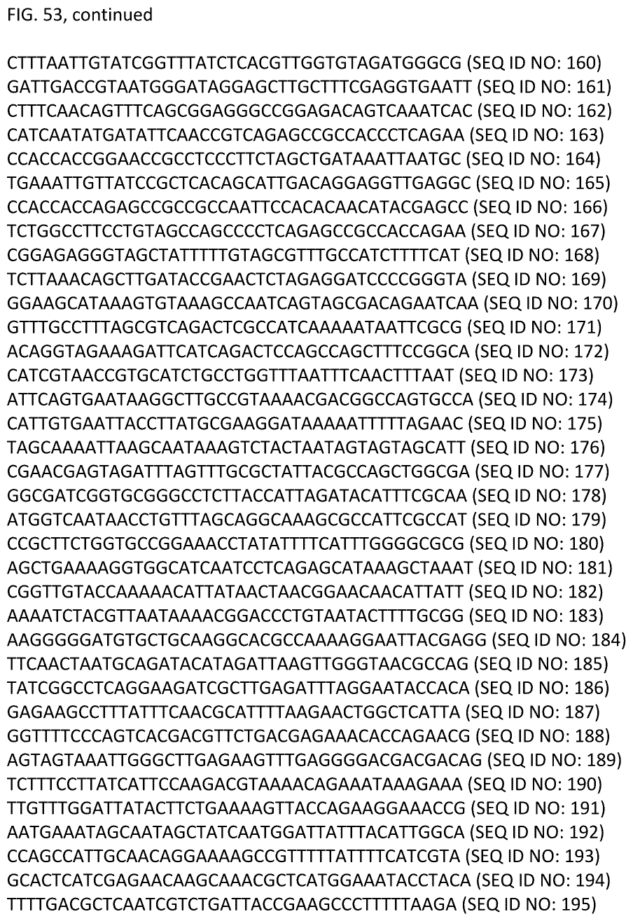

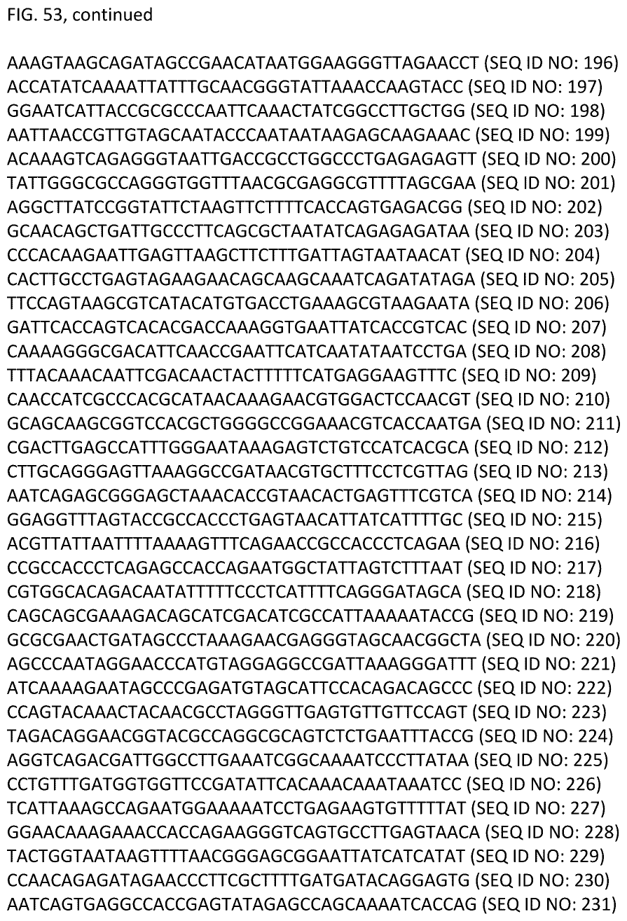

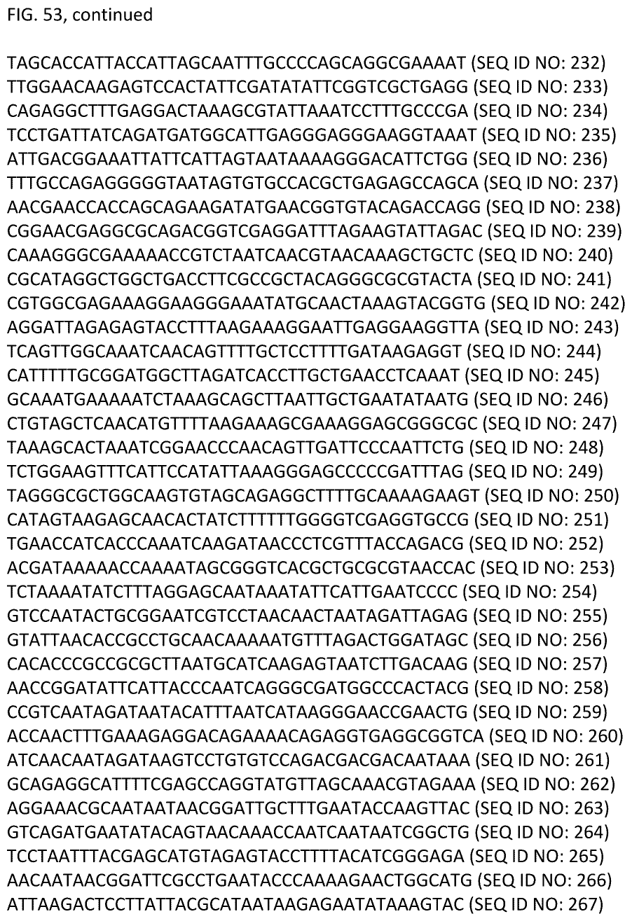

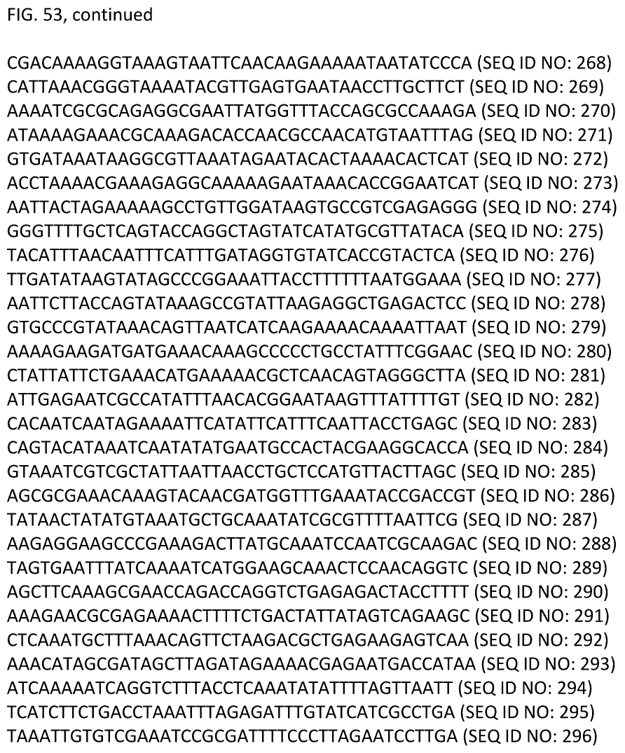

In some embodiments, the linker is selected from a (Gly-Gly-Gly-Gly-Ser) n sequence where n is from 1 to 8 (SEQ ID NO: 1), or a human C8 hinge sequence. It was found that a spacer was required to position the targeting molecule from the transmembrane domain. Engineered T cells comprising a CD19 CAR with linkers (4×GS linker (n=4), 3×GS (n=3) linker, or CD8 hinge) help engineered T cells release more IFN-γ when cocultured with Raji cells compared to engineered T cells with no linker. Moreover, it was surprisingly discovered that the linker length of the 4×GS (which consists of 20 amino acids) resulted in about the same effect as the CD8 hinge (which consists of about 43 amino acids), but both linkers exhibited slightly higher IFN-Y release compared to the 3×GS (which consisted of 15 amino acids), suggesting a plateau in the linker length on the IFN-γ release levels. As shown in FIG. 49, the engineered CD19 CAR vector comprised a leader sequence (L), CD19 single-chain variable fragment (scFv), linker, CD8 transmembrane domain (TM), CD28 intracellular domain (CD28) and CD3ζ signaling domain. The vectors encoding the four types of engineered CARs were transduced into human T cells to generate engineered T cells. CD19 positive human Raji cell lines were cocultured with engineered T cells. IFN-γ release of CAR-T cells was measured by ELISA. The results also confirm that, inter alia, engineered T cells comprising a scFv as the targeting molecule was activated in the presence of a target cell.

### Example 15. Engineered T Cells Comprising an Antibody are Activated in the Presence of a Target Tumor Cell

As shown in FIG. 50, engineered T cells comprising an antibody as the targeting molecule were chemically connected to a second oligonucleotide linked which was immobilized to a first oligonucleotide which was chemically connected to the adaptor protein of the engineered CAR.

MDA-MB-231 was a Programmed death-ligand 1 (PD-L1) positive human breast cancer cell line. FIG. 50A shows the strategy for the production of aPD-L1 engineered T cells. The aPD-L1 antibody was obtained from commercial vendors, and the aPD-L1 engineered T cells were generated by the methods described herein. As shown in FIG. 50B, the PD-L1 antibody (alpha-PD-L1) and PD-L1 antibody conjugated with polyT (Poly T alpha-PD-L1) exhibited different mobilities in SDS-PAGE. The mobility difference demonstrates that the engineered T cell comprising an antibody was generated. PD-L1 positive MDA-MB-231 cells activated PD-L1 DNA-CAR T cells.

MDA-MB-231 breast cells and anti-PD-L1 engineered T cells were incubated for 12 hours, after which T cell activation levels of luciferase was measured by relative luminescence units (RLU). In FIG. 50C, the second column shows that PD-L1 engineered T cells were activated by tumor cancer MDA-MB-231 cells. Only low levels of luciferase release were observed in Adaptor

Jurkat T cell alone, indicating that the engineered T cells comprising an antibody as the targeting molecule were specifically activated in the presence of the cancer tumor cell. Combined with the experiment described above demonstrating that an engineered T cell comprising an aptamer bound to a cancer cell target, these data demonstrate that engineered T cells comprising a genus of targeting molecules bind to their cognate ligands on cancer cells to activate said engineered T cells.

### Example 16. Engineered T Cells in a Mouse Model Kill Cancer Cells

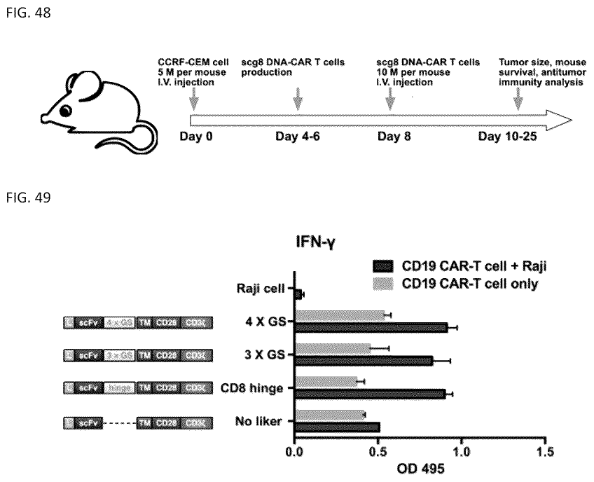

The NSG mouse model is used assess the in vivo anti-tumor effect of control and transduced engineered Jurkat T cells. The CCRF-CEM cell line was used, which expresses the PT7 tumor marker, to monitor tumor growth in vivo. 8-10-week-old male and female NSG mice are injected intraperitoneally with CCRF-CEM cancer cells resuspended in Matrigel (BD Biosciences). Tumors are induced by subcutaneous injection of 5×10⁶ tumor cells, and mice are treated by IV injection of ×10⁷ T cells as indicated in FIG. 48. For re-challenge experiments, mice are injected subcutaneously with 0.5×10⁶ cells in the flank opposite to the site of the previously rejected tumor. All experiments are randomized and blinded. Tumor growth and condition of mice are monitored every other day. For antitumoral efficacy 6-8 mice per group are used.

An engineered T cell comprising scg8 aptamers is generated using the methods described herein. The scg8 engineered T cells are used to eliminate CCFR-CEM cells in a NSG humanized mouse model. Before injected tumor cells, NSG mice are irradiated and injected with 100 million (M) human PBMC to create a human T cell friendly immune system. Before injecting tumor cells, NSG mice are irradiated and injected with 100 million (M) human PBMC to create a human T cell friendly immune system. After PBMC transplantation 30 days, mice are injected with PTK7 positive CCRF-CEM cells (Day 0). About 100 million engineered T cells comprising scg8 aptamers is injected at Day 8 to the cohort test mice. T cell survival and CCRF-CEM cells is analyzed by flow cytometry at day 10.

In addition, peripheral blood from engineered T cell adoptive transferred mice is collected for analysis. Engineered T cells are isolated by Dynabeads Untouched Human T Cells Isolation Kit. Using flow cytometry, T cell activation efficiency by INF-γ or exhausting by PD-1 staining is measured. Serum is used to quantify different cytokine release by activated T cells by ELISA.

The mouse tumor model is also used for evaluating DNA-CAR T cell function in vitro proliferation. Tumor samples are collected after adoptive transfer (from day 1 to day 10) to check T cell infiltration into the tumor by CD3 staining. Peripheral blood samples are also collected to analyze T cell activities by flow cytometry and IFNγ, IL-2 and PD-1 staining.

The expected results show that engineered T cells conjugated with targeting molecules traffic to solid tumor area and successfully reduce tumor volume and prolong survival mouse life. Mouse survival of each cohort is also measured which shows that the mice cohort treated with the engineered T cells exhibit a longer survival time than the control cohorts. The results confirm that engineered T cells are both activated and can kill cancer cells in the presence of a cancer cell. Targeting efficiency or engineered T cell trafficking may lower than expected, peptides and DNA nanostructures including aptamers and origami are further optimized to get better outcomes. Furthermore, since the engineered T cell is easy to customize, different combinations of targeting molecules, spacers domains, and/or targeting agents are tested to obtain higher T cell potency. In some embodiments, three or four targeting molecules can simultaneously be added to the engineered T cells, or multiple targeting molecules plus cytokines are added to the engineered T cells, or multiple targeting molecules plus checkpoint blockade are added to the engineered T cells.

All DNA and RNA sequences presented herein are oriented 5′->3′, unless noted otherwise.

Although the foregoing specification and examples fully disclose and enable this disclosure, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “including”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-35. (canceled)

36. A method of preparing an engineered T cell comprising:
(a) inserting a DNA sequence which encodes for the CAR polypeptide into a virus;
(b) contacting the virus with a T cell to form a viral-infused T cell;
(c) growing the viral-infused T cells to produce an adaptor T cell expressing the CAR polypeptide comprising an extracellular adaptor protein;
(d) isolating the adaptor T cell;
(e) contacting the isolated adaptor T cells with a first oligonucleotide functionalized with a cognate protein tag;
(f) forming a complex between the extracellular adaptor protein of the adaptor T cells with the cognate protein tag to form a first oligonucleotide-functionalized adaptor T cell;
(g) contacting the first oligonucleotide-functionalized adaptor T cell with a second oligonucleotide comprising a targeting agent under appropriate conditions to form a hybridization complex between a portion of the first linker oligonucleotide and a portion of the second linker oligonucleotide,
wherein the engineered T cell comprises:
(1) an expressed engineered chimeric antigen receptor (CAR) which comprises an extracellular adaptor protein selected from O6-alkylguanine-DNA alkyltransferase which reacts with O6-benzylguanine moiety, or O6-alkylguanine DNA alkyltransferase which reacts with O2-benzylcytosine moiety;
(2) a protein tag bound to said adaptor protein;
(3) a first oligonucleotide connected to said protein tag;
(4) a second oligonucleotide wherein a portion of the second oligonucleotide sequence is complementary to a portion of the first oligonucleotide sequence; and
(5) a targeting agent connected to the second oligonucleotide, wherein the targeting agent comprises one or a plurality of targeting molecules,
wherein the targeting agent comprises:
(I) a DNA origami nanostructure comprising a central polynucleotide strand and a first staple strand which comprises the second oligonucleotide sequence and a plurality of second staple strands which comprises one or a plurality of third distinct oligonucleotide sequences; and
(II) one or more targeting molecules connected to one or a plurality of fourth distinct oligonucleotide sequence(s),
wherein a portion of the third distinct oligonucleotide sequence is complementary to a portion of the fourth oligonucleotide sequence.

37. A method of activating an engineered T cell, the method comprising contacting a cancer cell with an engineered T cell, wherein the engineered T cell comprises:
(1) an expressed engineered chimeric antigen receptor (CAR) which comprises an extracellular adaptor protein selected from O6-alkylguanine-DNA alkyltransferase which reacts with O6-benzylguanine moiety, or O6-alkylguanine DNA alkyltransferase which reacts with 02-benzylcytosine moiety;
(2) a protein tag bound to said adaptor protein;
(3) a first oligonucleotide connected to said protein tag;
(4) a second oligonucleotide wherein a portion of the second oligonucleotide sequence is complementary to a portion of the first oligonucleotide sequence; and
(5) a targeting agent connected to the second oligonucleotide, wherein the targeting agent comprises one or a plurality of targeting molecules,
wherein the targeting agent comprises:
(I) a DNA origami nanostructure comprising a central polynucleotide strand and a first staple strand which comprises the second oligonucleotide sequence and a plurality of second staple strands which comprises one or a plurality of third distinct oligonucleotide sequences; and
(II) one or more targeting molecules connected to one or a plurality of fourth distinct oligonucleotide sequence(s),
wherein a portion of the third distinct oligonucleotide sequence is complementary to a portion of the fourth oligonucleotide sequence.

38. The method of any of claim 36 or 37, wherein the engineered T cell comprises an expressed engineered chimeric antigen receptor comprising:
(a) a signaling polypeptide domain;
(b) a transmembrane polypeptide domain;
(c) a spacer polypeptide domain;
(d) a costimulatory polypeptide domain; and
(e) an adaptor protein tag domain.

39. The method of claim 38, wherein the engineered T cell further comprises a fluorescent protein domain.

40. The method of claim 39, wherein the fluorescent protein domain is green fluorescent protein (GFP).

41. The method of claim 39, wherein the engineered T cell further comprises an antibiotic resistant gene.

42. The method of claim 39, wherein the signaling polypeptide domain is CD3ζ.

43. The method of claim 39, wherein the transmembrane polypeptide domain is CD8.

44. The method of claim 39, wherein the costimulatory domain is selected from: CD28, 4-1BB, OX-40, and combinations thereof.

45. The method of claim 39, wherein the spacer polypeptide domain is a repeat of the sequence (Gly-Gly-Gly-Gly-Ser) n, (SEQ ID NO: 1) where n is an integer selected from 1 to 8.

46. The method of claim 39, wherein the spacer polypeptide domain is a human CD8 hinge sequence comprising the sequence of SEQ ID NO: 4.

47. The method of claim 39, wherein the signaling polypeptide domain is CD3ζ, the transmembrane polypeptide domain is CD8, the costimulatory domain is 4-1BB, the spacer polypeptide domain is (Gly-Gly-Gly-Gly-Ser) 4, (SEQ ID NO: 2) and the adaptor protein tag domain is O6-methylguanine-DNA methyltransferase adaptor protein.

48. The method of claim 36 or 37, wherein the wherein the targeting agent comprises one or a plurality of targeting molecule selected from: an aptamer, a synbody, and an antibody or fragment thereof.

49. The method of claim 48, wherein the antibody fragment is a ScFv.

50. The method of claim 36 or 37, wherein one of the one or a plurality of targeting molecules comprises a matrix metalloproteinase

51. The method of claim 36 or 37, wherein one of the one or a plurality of targeting molecules comprises a cytokine or chemokine.

52. The method of claim 36 or 37, wherein one of the one or a plurality of targeting molecules comprises an inhibitory pathway overcoming agent.

53. The method of claim 52, wherein the inhibitory pathway overcoming agent is selected from an anti-PD-1L antibody, an anti-PD-1L aptamer, an anti-CTLA4 antibody, or an anti-CTLA4 aptamer.

54. The method, of claim 53, wherein the aptamer is scg8 having SEQ ID NO: 10.

55. The method of claim 36 or 37, wherein the T cell is selected from a natural killer T cell, a regulatory T cell, a helper T cell, a cytotoxic T cell, a memory T cell, a gamma delta T cell and a mucosal invariant T cell.

56. The method of claim 36, wherein the virus is selected from a lentivirus, retrovirus or adeno-associated virus.

57. The method of claim 56, wherein the cancer cell is selected from a hematological cancer or a tumor cell.

58. The method of claim 57, wherein the cancer cell is selected from a hematological cancer or a tumor cell.

59. The method of claim 58, wherein the hematological cancer is a T lymphoblastoid cell.

60. The method of claim 59, wherein the tumor cell is selected from a breast cancer cell or a brain cancer cell.


SEQUENCE LISTING (296 sequences)

SEQ ID NO: 1 - protein, 40 aa, synthetic construct
  SITE 1..40: note = /note="This sequence may encompass 1-8 'Gly Gly Gly Gly Ser' repeating units"
  REGION 1..40: note = source = /note="Description of Artificial Sequence: Synthetic polypeptide"
GGGGSGGGGSGGGGSGGGGSGGGGSGGGGSGGGGSGGGGS

SEQ ID NO: 2 - protein, 20 aa, synthetic construct
  REGION 1..20: note = source = /note="Description of Artificial Sequence: Synthetic peptide"
GGGGSGGGGSGGGGSGGGGS

SEQ ID NO: 3 - protein, 100 aa, synthetic construct
  SITE 1..100: note = /note="This sequence may encompass 1-20 'Gly Gly Gly Gly Ser' repeating units"
  REGION 1..100: note = source = /note="Description of Artificial Sequence: Synthetic polypeptide"
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 4 - protein, 45 aa, Homo sapiens
TTTPAPRPPTPAPTIASQPLSLRPEACRPAAGGAVHTRGLDFACD

SEQ ID NO: 5 - DNA, 45 bp, synthetic construct
  misc_feature 1..45: note = source = /note="Description of Artificial Sequence: Synthetic oligonucleotide"
  source 1..45: mol_type = other DNA
acgggccacatcaactcattgatagacaatgcgtccactgcccgt

SEQ ID NO: 6 - DNA, 25 bp, synthetic construct
  misc_feature 1..25: note = source = /note="Description of Artificial Sequence: Synthetic oligonucleotide"
  source 1..25: mol_type = other DNA
gcagttgatcctttggataccctgg

SEQ ID NO: 7 - DNA, 34 bp, synthetic construct
  misc_feature 1..34: note = source = /note="Description of Artificial Sequence: Synthetic oligonucleotide"
  source 1..34: mol_type = other DNA
gcagttgatcctttggataccctggtttttaaaa

SEQ ID NO: 8 - DNA, 72 bp, synthetic construct
  misc_feature 1..72: note = source = /note="Description of Artificial Sequence: Synthetic oligonucleotide"
  source 1..72: mol_type = other DNA
gggagacaagaataaacgctcaagcagttgatcctttggataccctggttcgacaggaggctcacaacaggc

SEQ ID NO: 9 - DNA, 76 bp, synthetic construct
  misc_feature 1..76: note = source = /note="Description of Artificial Sequence: Synthetic oligonucleotide"
  source 1..76: mol_type = other DNA
taccagtgcgatgctcagtgccgtttcttctctttcgctttttttgcttttgagcatgctgacgcattcggttgac

SEQ ID NO: 10 - DNA, 41 bp, synthetic construct
  misc_feature 1..41: note = source = /note="Description of Artificial Sequence: Synthetic oligonucleotide"
  source 1..41: mol_type = other DNA
atctaactgctgcgccgccgggaaaatactgtacggttaga

SEQ ID NO: 11 - DNA, 55 bp, synthetic construct
  misc_feature 1..55: note = source = /note="Description of Artificial Sequence: Synthetic oligonucleotide"
  source 1..55: mol_type = other DNA
aaaaaaaaaaaaaaacgtgcagtacgccaacctttctcatgcgctgcccctctta

SEQ ID NO: 12 - protein, 5 aa, synthetic construct
  REGION 1..5: note = source = /note="Description of Artificial Sequence: Synthetic peptide"
LPETG

SEQ ID NO: 13 - protein, 315 aa, Rhodopseudomonas palustris
AEGSVARQPDLLTCDDEPIHIPGAIQPHGLLLALAADMTIVAGSDNLPELTGLAIGALIGRSAADVFDSETHNRLTIALAEPGAAVGAPITVGFTMRKDAGFIGSWHRHDQLIFLELEPPQRDVAEPQAFFRRTNSAIRRLQAAETLESACAAAAQEVRKITGFDRVMIYRFASDFSGEVIAEDRCAEVESKLGLHYPASTVPAQARRLYTINPVRIIPDINYRPVPVTPDLNPVTGRPIDLSFAILRSVSPVHLEFMRNIGMHGTMSISILRGERLWGLIVCHHRTPYYVDLDGRQACELVAQVLAWQIGVMEE

SEQ ID NO: 14 - protein, 16 aa, synthetic construct
  REGION 1..16: note = source = /note="Description of Artificial Sequence: Synthetic peptide"
GGGSGGGSGGGSGGGS

SEQ ID NO: 15 - DNA, 9396 bp, synthetic construct
  misc_feature 1..9396: note = source = /note="Description of Artificial Sequence: Synthetic polynucleotide"
  source 1..9396: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacatgtccaatatgaccgccatgttgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaatggccctgcctgtgacagccctgctgctgcctctggctctgctgctgcatgccgctagacccgacaaagactgcgaaatgaagcgcaccaccctggatagccctctgggcaagctggaactgtctgggtgcgaacagggcctgcaccgtatcatcttcctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctacagccacctggccgccctggccggcaatcccgccgccaccgccgccgtgaaaaccgccctgagcggaaatcccgtgcccattctgatcccctgccaccgggtggtgcagggcgacctggacgtggggggctacgagggcgggctcgccgtgaaagagtggctgctggcccacgagggccacagactgggcaagcctgggctgggttcaggtggaggcggttcaggtggaggcggttcaggtggaggcggtatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgacgcgtacgcggccgctcgagaatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtagtccggactcagagtttgggtaggaagcggagctactaacttcagcctgctgaagcaggctggagacgtggaggagaaccctggacctatgaccgagtacaagcccacggtgcgcctcgccacccgcgacgacgtccccagggccgtacgcaccctcgccgccgcgttcgccgactaccccgccacgcgccacaccgtcgatccggatcgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgtacacccaaacggccggccgcggtctgtacaagtaggattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 16 - DNA, 42 bp, synthetic construct
  misc_feature 1..42: note = source = /note="Description of Artificial Sequence: Synthetic oligonucleotide"
  source 1..42: mol_type = other DNA
gaaaattcataagtaagcgtcatacatggcttagacgggaga

SEQ ID NO: 17 - DNA, 42 bp, synthetic construct
  misc_feature 1..42: note = source = /note="Description of Artificial Sequence: Synthetic oligonucleotide"
  source 1..42: mol_type = other DNA
atattcacaaaataaaaacagggaagcgcattttgatgatac

SEQ ID NO: 18 - DNA, 42 bp, synthetic construct
  misc_feature 1..42: note = source = /note="Description of Artificial Sequence: Synthetic oligonucleotide"
  source 1..42: mol_type = other DNA
aggagtgtactaataaacagccatattatttaattggccttg

SEQ ID NO: 19 - DNA, 42 bp, synthetic construct
  misc_feature 1..42: note = source = /note="Description of Artificial Sequence: Synthetic oligonucleotide"
  source 1..42: mol_type = other DNA
gccagttacaaggtaataagttttaacggggtgtcctgaaca

SEQ ID NO: 20 - DNA, 42 bp, synthetic construct
  misc_feature 1..42: note = source = /note="Description of Artificial Sequence: Synthetic oligonucleotide"
  source 1..42: mol_type = other DNA
gcagaacgcgcgaggttgaggcaggtcagacgtcccaatcca

SEQ ID NO: 21 - DNA, 42 bp, synthetic construct
  misc_feature 1..42: note = source = /note="Description of Artificial Sequence: Synthetic oligonucleotide"
  source 1..42: mol_type = other DNA
aataagaaacggacttgagccatttgggaattttcagctaat

SEQ ID NO: 22 - DNA, 42 bp, synthetic construct
  misc_feature 1..42: note = source = /note="Description of Artificial Sequence: Synthetic oligonucleotide"
  source 1..42: mol_type = other DNA
gagagaataaccaaataaatcctcattaaagcaaaagggcga

SEQ ID NO: 23 - DNA, 42 bp, synthetic construct
  misc_feature 1..42: note = source = /note="Description of Artificial Sequence: Synthetic oligonucleotide"
  source 1..42: mol_type = other DNA
agcattgacagctgtttatcaacaatagataacagtgccttg

SEQ ID NO: 24 - DNA, 42 bp, synthetic construct
  misc_feature 1..42: note = source = /note="Description of Artificial Sequence: Synthetic oligonucleotide"
  source 1..42: mol_type = other DNA
agtaacagtgcccagtaataagagaatataaaagccgccgcc

SEQ ID NO: 25 - DNA, 42 bp, synthetic construct
  misc_feature 1..42: note = source = /note="Description of Artificial Sequence: Synthetic oligonucleotide"
  source 1..42: mol_type = other DNA
gcattttcgagccgtataaacagttaatgccctatcaaaatc

SEQ ID NO: 26 - DNA, 42 bp, synthetic construct
  misc_feature 1..42: note = source = /note="Description of Artificial Sequence: Synthetic oligonucleotide"
  source 1..42: mol_type = other DNA
attaagacgctcgccaccagaaccaccaccaggtaccgacaa

SEQ ID NO: 27 - DNA, 42 bp, synthetic construct
  misc_feature 1..42: note = source = /note="Description of Artificial Sequence: Synthetic oligonucleotide"
  source 1..42: mol_type = other DNA
aaggtaaagtaagcaccattaccattagcaaggatagcttag

SEQ ID NO: 28 - DNA, 42 bp, synthetic construct
  misc_feature 1..42: note = source = /note="Description of Artificial Sequence: Synthetic oligonucleotide"
  source 1..42: mol_type = other DNA
taccgttcctggtttaccagcgccaaagaccagaatggaaag

SEQ ID NO: 29 - DNA, 42 bp, synthetic construct
  misc_feature 1..42: note = source = /note="Description of Artificial Sequence: Synthetic oligonucleotide"
  source 1..42: mol_type = other DNA
cattcaaccgattgacggaaattattcattaaagcctttaca

SEQ ID NO: 30 - DNA, 40 bp, synthetic construct
  misc_feature 1..40: note = source = /note="Description of Artificial Sequence: Synthetic oligonucleotide"
  source 1..40: mol_type = other DNA
gaaaatagcaggtgaattatcaccgtcaccattttttgtt

SEQ ID NO: 31 - DNA, 42 bp, synthetic construct
  misc_feature 1..42: note = source = /note="Description of Artificial Sequence: Synthetic oligonucleotide"
  source 1..42: mol_type = other DNA
gcaaagacaccgtaaatgaattttctgtatggtaattgagcg

SEQ ID NO: 32 - DNA, 42 bp, synthetic construct
  misc_feature 1..42: note = source = /note="Description of Artificial Sequence: Synthetic oligonucleotide"
  source 1..42: mol_type = other DNA
tagcattccacaccctgaacaaagtcagaggggattttgcta

SEQ ID NO: 33 - DNA, 42 bp, synthetic construct
  misc_feature 1..42: note = source = /note="Description of Artificial Sequence: Synthetic oligonucleotide"
  source 1..42: mol_type = other DNA
aacaactttcacgctaacgagcgtctttccagacaacgcctg

SEQ ID NO: 34 - DNA, 42 bp, synthetic construct
  misc_feature 1..42: note = source = /note="Description of Artificial Sequence: Synthetic oligonucleotide"
  source 1..42: mol_type = other DNA
aatcttaccaaacagtttcagcggagtgagaaatgtagaaac

SEQ ID NO: 35 - DNA, 42 bp, synthetic construct
  misc_feature 1..42: note = source = /note="Description of Artificial Sequence: Synthetic oligonucleotide"
  source 1..42: mol_type = other DNA
agaaaaataatgtttcgtcaccagtacaaactagcctaattt

SEQ ID NO: 36 - DNA, 42 bp, synthetic construct
  misc_feature 1..42: note = source = /note="Description of Artificial Sequence: Synthetic oligonucleotide"
  source 1..42: mol_type = other DNA
attaactgaacagacagccctcatagttagcgacaatcaata

SEQ ID NO: 37 - DNA, 40 bp, synthetic construct
  misc_feature 1..40: note = source = /note="Description of Artificial Sequence: Synthetic oligonucleotide"
  source 1..40: mol_type = other DNA
aacaacatgagagccagcaaaatcaccagtattctgtcca

SEQ ID NO: 38 - DNA, 42 bp, synthetic construct
  misc_feature 1..42: note = source = /note="Description of Artificial Sequence: Synthetic oligonucleotide"
  source 1..42: mol_type = other DNA
cgtaacactgaatcccatcctaatttacgagctagaaaggaa

SEQ ID NO: 39 - DNA, 42 bp, synthetic construct
  misc_feature 1..42: note = source = /note="Description of Artificial Sequence: Synthetic oligonucleotide"
  source 1..42: mol_type = other DNA
caactaaaggattaacaacgccaacatgtaatacccatgtac

SEQ ID NO: 40 - DNA, 42 bp, synthetic construct
  misc_feature 1..42: note = source = /note="Description of Artificial Sequence: Synthetic oligonucleotide"
  source 1..42: mol_type = other DNA
aatcgccatatattgcgaataataattttttcgcttaggttg

SEQ ID NO: 41 - DNA, 42 bp, synthetic construct
  misc_feature 1..42: note = source = /note="Description of Artificial Sequence: Synthetic oligonucleotide"
  source 1..42: mol_type = other DNA
ataggtctgaggggatagcaagcccaataggattaggcagag

SEQ ID NO: 42 - DNA, 42 bp, synthetic construct
  misc_feature 1..42: note = source = /note="Description of Artificial Sequence: Synthetic oligonucleotide"
  source 1..42: mol_type = other DNA
cagacgttaacggaataagtttattttgtctaacgatctaaa

SEQ ID NO: 43 - DNA, 40 bp, synthetic construct
  misc_feature 1..40: note = source = /note="Description of Artificial Sequence: Synthetic oligonucleotide"
  source 1..40: mol_type = other DNA
tcgcccacgcagccattgcaacaggaaaaatgcgccgaca

SEQ ID NO: 44 - DNA, 42 bp, synthetic construct
  misc_feature 1..42: note = source = /note="Description of Artificial Sequence: Synthetic oligonucleotide"
  source 1..42: mol_type = other DNA
cctcattttcaagactacctttttaacctccgacgttgaaaa

SEQ ID NO: 45 - DNA, 42 bp, synthetic construct
  misc_feature 1..42: note = source = /note="Description of Artificial Sequence: Synthetic oligonucleotide"
  source 1..42: mol_type = other DNA
tctccaaaaaatgaattaccttttttaatggagagccaccac

SEQ ID NO: 46 - DNA, 40 bp, synthetic construct
  misc_feature 1..40: note = source = /note="Description of Artificial Sequence: Synthetic oligonucleotide"
  source 1..40: mol_type = other DNA
atataagtatttgacgctcaatcgtctgaagataagtgcc

SEQ ID NO: 47 - DNA, 42 bp, synthetic construct
  misc_feature 1..42: note = source = /note="Description of Artificial Sequence: Synthetic oligonucleotide"
  source 1..42: mol_type = other DNA
accctcagagcgagaagagtcaatagtgaattcctgcctatt

SEQ ID NO: 48 - DNA, 42 bp, synthetic construct
  misc_feature 1..42: note = source = /note="Description of Artificial Sequence: Synthetic oligonucleotide"
  source 1..42: mol_type = other DNA
tcggaacctattgtgagtgaataaccttgcttcagagccacc

SEQ ID NO: 49 - DNA, 40 bp, synthetic construct
  misc_feature 1..40: note = source = /note="Description of Artificial Sequence: Synthetic oligonucleotide"
  source 1..40: mol_type = other DNA
cgtttgccaattcaccagtcacacgaccagttcggtcata

SEQ ID NO: 50 - DNA, 40 bp, synthetic construct
  misc_feature 1..40: note = source = /note="Description of Artificial Sequence: Synthetic oligonucleotide"
  source 1..40: mol_type = other DNA
aaacatagcgccggaaacgtcaccaatgaataattttccc

SEQ ID NO: 51 - DNA, 42 bp, synthetic construct
  misc_feature 1..42: note = source = /note="Description of Artificial Sequence: Synthetic oligonucleotide"
  source 1..42: mol_type = other DNA
acaatttcattaaggctccaaaaggagccttttatacttctg

SEQ ID NO: 52 - DNA, 40 bp, synthetic construct
  misc_feature 1..40: note = source = /note="Description of Artificial Sequence: Synthetic oligonucleotide"
  source 1..40: mol_type = other DNA
gaacaaagaatcagtagcgacagaatcaagttgagtaaca

SEQ ID NO: 53 - DNA, 42 bp, synthetic construct
  misc_feature 1..42: note = source = /note="Description of Artificial Sequence: Synthetic oligonucleotide"
  source 1..42: mol_type = other DNA
gataatacatttgctttcgaggtgaatttcttagccctaaaa

SEQ ID NO: 54 - DNA, 40 bp, synthetic construct
  misc_feature 1..40: note = source = /note="Description of Artificial Sequence: Synthetic oligonucleotide"
  source 1..40: mol_type = other DNA
acagagatatagcgcgttttcatcggcatttaataaaagg

SEQ ID NO: 55 - DNA, 42 bp, synthetic construct
  misc_feature 1..42: note = source = /note="Description of Artificial Sequence: Synthetic oligonucleotide"
  source 1..42: mol_type = other DNA
attttaaaagttttgcctttagcgtcagactggaacccttct

SEQ ID NO: 56 - DNA, 42 bp, synthetic construct
  misc_feature 1..42: note = source = /note="Description of Artificial Sequence: Synthetic oligonucleotide"
  source 1..42: mol_type = other DNA
gacctgaaagccggaaccagagccaccaccggaacgttatta

SEQ ID NO: 57 - DNA, 42 bp, synthetic construct
  misc_feature 1..42: note = source = /note="Description of Artificial Sequence: Synthetic oligonucleotide"
  source 1..42: mol_type = other DNA
atcaaaatcacgtaagaatacgtggcacagacggttttgctc

SEQ ID NO: 58 - DNA, 42 bp, synthetic construct
  misc_feature 1..42: note = source = /note="Description of Artificial Sequence: Synthetic oligonucleotide"
  source 1..42: mol_type = other DNA
agtaccaggcgatggattatttacattggcagtcttttcata

SEQ ID NO: 59 - DNA, 42 bp, synthetic construct
  misc_feature 1..42: note = source = /note="Description of Artificial Sequence: Synthetic oligonucleotide"
  source 1..42: mol_type = other DNA
aattcgacaacgagaaggattaggattagcggaatatttttg

SEQ ID NO: 60 - DNA, 42 bp, synthetic construct
  misc_feature 1..42: note = source = /note="Description of Artificial Sequence: Synthetic oligonucleotide"
  source 1..42: mol_type = other DNA
aatggctattaccgtactcaggaggtttagtactttacaaac

SEQ ID NO: 61 - DNA, 42 bp, synthetic construct
  misc_feature 1..42: note = source = /note="Description of Artificial Sequence: Synthetic oligonucleotide"
  source 1..42: mol_type = other DNA
taggtgtatcagtctttaatgcgcgaactgataaacagcttg

SEQ ID NO: 62 - DNA, 42 bp, synthetic construct
  misc_feature 1..42: note = source = /note="Description of Artificial Sequence: Synthetic oligonucleotide"
  source 1..42: mol_type = other DNA
ataccgatagtcgctcatggaaatacctacattagcccggaa

SEQ ID NO: 63 - DNA, 42 bp, synthetic construct
  misc_feature 1..42: note = source = /note="Description of Artificial Sequence: Synthetic oligonucleotide"
  source 1..42: mol_type = other DNA
gtttatcagcttgaggatttagaagtattagaccgccaccct

SEQ ID NO: 64 - DNA, 42 bp, synthetic construct
  misc_feature 1..42: note = source = /note="Description of Artificial Sequence: Synthetic oligonucleotide"
  source 1..42: mol_type = other DNA
cagaaccgccatataatcctgattgtttggataattgtatcg

SEQ ID NO: 65 - DNA, 42 bp, synthetic construct
  misc_feature 1..42: note = source = /note="Description of Artificial Sequence: Synthetic oligonucleotide"
  source 1..42: mol_type = other DNA
agactcctcaatcgtattaaatcctttgcccgaaccgcctcc

SEQ ID NO: 66 - DNA, 42 bp, synthetic construct
  misc_feature 1..42: note = source = /note="Description of Artificial Sequence: Synthetic oligonucleotide"
  source 1..42: mol_type = other DNA
ctcagagccgctatcatcatattcctgattattaagaggctg

SEQ ID NO: 67 - DNA, 42 bp, synthetic construct
  misc_feature 1..42: note = source = /note="Description of Artificial Sequence: Synthetic oligonucleotide"
  source 1..42: mol_type = other DNA
tcgctattaataccatcgatagcagcaccgtaaaccaccaga

SEQ ID NO: 68 - DNA, 42 bp, synthetic construct
  misc_feature 1..42: note = source = /note="Description of Artificial Sequence: Synthetic oligonucleotide"
  source 1..42: mol_type = other DNA
aggagcggaatcaccctcagaaccgccaccctctgtaaatcg

SEQ ID NO: 69 - DNA, 42 bp, synthetic construct
  misc_feature 1..42: note = source = /note="Description of Artificial Sequence: Synthetic oligonucleotide"
  source 1..42: mol_type = other DNA
aaatcaatatatattctgaaacatgaaagtatcagatgatgg

SEQ ID NO: 70 - DNA, 42 bp, synthetic construct
  misc_feature 1..42: note = source = /note="Description of Artificial Sequence: Synthetic oligonucleotide"
  source 1..42: mol_type = other DNA
caattcatcaaccctcagaaccgccaccctcaaacagtacat

SEQ ID NO: 71 - DNA, 42 bp, synthetic construct
  misc_feature 1..42: note = source = /note="Description of Artificial Sequence: Synthetic oligonucleotide"
  source 1..42: mol_type = other DNA
ggttatataaccggctacagaggctttgaggacttaattgag

SEQ ID NO: 72 - DNA, 40 bp, synthetic construct
  misc_feature 1..40: note = source = /note="Description of Artificial Sequence: Synthetic oligonucleotide"
  source 1..40: mol_type = other DNA
taaggcgttcccaattctgcgaacgagtagtgaaataccg

SEQ ID NO: 73 - DNA, 42 bp, synthetic construct
  misc_feature 1..42: note = source = /note="Description of Artificial Sequence: Synthetic oligonucleotide"
  source 1..42: mol_type = other DNA
ttaattgctaacgcaataataacggaatacaggtcatttttg

SEQ ID NO: 74 - DNA, 42 bp, synthetic construct
  misc_feature 1..42: note = source = /note="Description of Artificial Sequence: Synthetic oligonucleotide"
  source 1..42: mol_type = other DNA
taatttcatctacttcaaatatcgcgttttaatcataattac

SEQ ID NO: 75 - DNA, 42 bp, synthetic construct
  misc_feature 1..42: note = source = /note="Description of Artificial Sequence: Synthetic oligonucleotide"
  source 1..42: mol_type = other DNA
tagaaaaagccgtttaccagacgacgataaaaatattttagt

SEQ ID NO: 76 - DNA, 42 bp, synthetic construct
  misc_feature 1..42: note = source = /note="Description of Artificial Sequence: Synthetic oligonucleotide"
  source 1..42: mol_type = other DNA
ttgagatttagactccttattacgcagtatattattacaggt

SEQ ID NO: 77 - DNA, 42 bp, synthetic construct
  misc_feature 1..42: note = source = /note="Description of Artificial Sequence: Synthetic oligonucleotide"
  source 1..42: mol_type = other DNA
aagacaaagaataatcattgtgaattaccttatacaaattct

SEQ ID NO: 78 - DNA, 42 bp, synthetic construct
  misc_feature 1..42: note = source = /note="Description of Artificial Sequence: Synthetic oligonucleotide"
  source 1..42: mol_type = other DNA
taccagtataatccatgttacttagccggaacatccaatcgc

SEQ ID NO: 79 - DNA, 42 bp, synthetic construct
  misc_feature 1..42: note = source = /note="Description of Artificial Sequence: Synthetic oligonucleotide"
  source 1..42: mol_type = other DNA
ctttgacccatacataaaggtggcaacataggcaaaagaata

SEQ ID NO: 80 - DNA, 40 bp, synthetic construct
  misc_feature 1..40: note = source = /note="Description of Artificial Sequence: Synthetic oligonucleotide"
  source 1..40: mol_type = other DNA
aaccgaggagaatataatgctgtagctcaagccgaacaaa

SEQ ID NO: 81 - DNA, 42 bp, synthetic construct
  misc_feature 1..42: note = source = /note="Description of Artificial Sequence: Synthetic oligonucleotide"
  source 1..42: mol_type = other DNA
ctaatatcagagcaccaacctaaaacgaaagataaaagaaac

SEQ ID NO: 82 - DNA, 42 bp, synthetic construct
  misc_feature 1..42: note = source = /note="Description of Artificial Sequence: Synthetic oligonucleotide"
  source 1..42: mol_type = other DNA
ccactacgaaggagataacccacaagaattgaaaacaaagta

SEQ ID NO: 83 - DNA, 42 bp, synthetic construct
  misc_feature 1..42: note = source = /note="Description of Artificial Sequence: Synthetic oligonucleotide"
  source 1..42: mol_type = other DNA
caacggagattctattttgcacccagctacaaatacgtaatg

SEQ ID NO: 84 - DNA, 40 bp, synthetic construct
  misc_feature 1..40: note = source = /note="Description of Artificial Sequence: Synthetic oligonucleotide"
  source 1..40: mol_type = other DNA
tagaaggctaagtacggtgtctggaagtttcccaatagca

SEQ ID NO: 85 - DNA, 42 bp, synthetic construct
  misc_feature 1..42: note = source = /note="Description of Artificial Sequence: Synthetic oligonucleotide"
  source 1..42: mol_type = other DNA
caatcaataatagtttccattaaacgggtaaattttatcctg

SEQ ID NO: 86 - DNA, 42 bp, synthetic construct
  misc_feature 1..42: note = source = /note="Description of Artificial Sequence: Synthetic oligonucleotide"
  source 1..42: mol_type = other DNA
tttcatgaggacggctgtctttccttatcattgtgtcgaaat

SEQ ID NO: 87 - DNA, 42 bp, synthetic construct
  misc_feature 1..42: note = source = /note="Description of Artificial Sequence: Synthetic oligonucleotide"
  source 1..42: mol_type = other DNA
ccgcgacctgcagccaacgctcaacagtagggctaaagactt

SEQ ID NO: 88 - DNA, 42 bp, synthetic construct
  misc_feature 1..42: note = source = /note="Description of Artificial Sequence: Synthetic oligonucleotide"
  source 1..42: mol_type = other DNA
aagattagttgtgtatcatcgcctgataaattccaagaacgg

SEQ ID NO: 89 - DNA, 42 bp, synthetic construct
  misc_feature 1..42: note = source = /note="Description of Artificial Sequence: Synthetic oligonucleotide"
  source 1..42: mol_type = other DNA
gtattaaaccaattataccagtcaggacgttggccttaaatc

SEQ ID NO: 90 - DNA, 42 bp, synthetic construct
  misc_feature 1..42: note = source = /note="Description of Artificial Sequence: Synthetic oligonucleotide"
  source 1..42: mol_type = other DNA
agaactggctcagtaccgcactcatcgagaacagcaacacta

SEQ ID NO: 91 - DNA, 42 bp, synthetic construct
  misc_feature 1..42: note = source = /note="Description of Artificial Sequence: Synthetic oligonucleotide"
  source 1..42: mol_type = other DNA
tcataaccctctgtttagtatcatatgcgttatgcgatttta

SEQ ID NO: 92 - DNA, 42 bp, synthetic construct
  misc_feature 1..42: note = source = /note="Description of Artificial Sequence: Synthetic oligonucleotide"
  source 1..42: mol_type = other DNA
agcgaacctccggaattacgaggcatagtaagaagcaagccg

SEQ ID NO: 93 - DNA, 42 bp, synthetic construct
  misc_feature 1..42: note = source = /note="Description of Artificial Sequence: Synthetic oligonucleotide"
  source 1..42: mol_type = other DNA
tttttattttcgaccggaagcaaactccaacagaggcgtttt

SEQ ID NO: 94 - DNA, 42 bp, synthetic construct
  misc_feature 1..42: note = source = /note="Description of Artificial Sequence: Synthetic oligonucleotide"
  source 1..42: mol_type = other DNA
aaagcgaaccaatcgtaggaatcattaccgcgcattccatat

SEQ ID NO: 95 - DNA, 42 bp, synthetic construct
  misc_feature 1..42: note = source = /note="Description of Artificial Sequence: Synthetic oligonucleotide"
  source 1..42: mol_type = other DNA
aacagttgattaaataagaataaacaccggaattcgagcttc

SEQ ID NO: 96 - DNA, 42 bp, synthetic construct
  misc_feature 1..42: note = source = /note="Description of Artificial Sequence: Synthetic oligonucleotide"
  source 1..42: mol_type = other DNA
atatgcaactatatccggtattctaagaacgcggtcaggatt

SEQ ID NO: 97 - DNA, 42 bp, synthetic construct
  misc_feature 1..42: note = source = /note="Description of Artificial Sequence: Synthetic oligonucleotide"
  source 1..42: mol_type = other DNA
agagagtaccttttaagaaaagtaagcagatacatgttttaa

SEQ ID NO: 98 - DNA, 42 bp, synthetic construct
  misc_feature 1..42: note = source = /note="Description of Artificial Sequence: Synthetic oligonucleotide"
  source 1..42: mol_type = other DNA
taacgccaaaacgacttgcgggaggttttgaaggaagaaaaa

SEQ ID NO: 99 - DNA, 42 bp, synthetic construct
  misc_feature 1..42: note = source = /note="Description of Artificial Sequence: Synthetic oligonucleotide"
  source 1..42: mol_type = other DNA
tctacgttaataagaaacaatgaaatagcaattgcagataca

SEQ ID NO: 100 - DNA, 42 bp, synthetic construct
  misc_feature 1..42: note = source = /note="Description of Artificial Sequence: Synthetic oligonucleotide"
  source 1..42: mol_type = other DNA
gtagaaaatacccagcgattataccaagcgcggttaagccca

SEQ ID NO: 101 - DNA, 42 bp, synthetic construct
  misc_feature 1..42: note = source = /note="Description of Artificial Sequence: Synthetic oligonucleotide"
  source 1..42: mol_type = other DNA
ataataagagcaaaacgaactaacggaacaacgttagcaaac

SEQ ID NO: 102 - DNA, 42 bp, synthetic construct
  misc_feature 1..42: note = source = /note="Description of Artificial Sequence: Synthetic oligonucleotide"
  source 1..42: mol_type = other DNA
tggcatgattaaggaataccacattcaactaaagctatctta

SEQ ID NO: 103 - DNA, 42 bp, synthetic construct
  misc_feature 1..42: note = source = /note="Description of Artificial Sequence: Synthetic oligonucleotide"
  source 1..42: mol_type = other DNA
ccgaagcccttttaattgctccttttgataagccaaaagaac

SEQ ID NO: 104 - DNA, 42 bp, synthetic construct
  misc_feature 1..42: note = source = /note="Description of Artificial Sequence: Synthetic oligonucleotide"
  source 1..42: mol_type = other DNA
aagcccgaaagtctgacctaaatttaatggttatttagtttg

SEQ ID NO: 105 - DNA, 42 bp, synthetic construct
  misc_feature 1..42: note = source = /note="Description of Artificial Sequence: Synthetic oligonucleotide"
  source 1..42: mol_type = other DNA
accattagatagattgctttgaataccaagttgattaagagg

SEQ ID NO: 106 - DNA, 40 bp, synthetic construct
  misc_feature 1..40: note = source = /note="Description of Artificial Sequence: Synthetic oligonucleotide"
  source 1..40: mol_type = other DNA
taaacagtttttgattagtaataacatcaccattgaatcc

SEQ ID NO: 107 - DNA, 42 bp, synthetic construct
  misc_feature 1..42: note = source = /note="Description of Artificial Sequence: Synthetic oligonucleotide"
  source 1..42: mol_type = other DNA
aatttcaacttcgcgagaaaactttttcaaataccaaaatag

SEQ ID NO: 108 - DNA, 42 bp, synthetic construct
  misc_feature 1..42: note = source = /note="Description of Artificial Sequence: Synthetic oligonucleotide"
  source 1..42: mol_type = other DNA
cgagaggctttttattcatttcaattacctgagagatggttt

SEQ ID NO: 109 - DNA, 40 bp, synthetic construct
  misc_feature 1..40: note = source = /note="Description of Artificial Sequence: Synthetic oligonucleotide"
  source 1..40: mol_type = other DNA
attcattacaactatcggccttgctggtaaagtaatcttg

SEQ ID NO: 110 - DNA, 42 bp, synthetic construct
  misc_feature 1..42: note = source = /note="Description of Artificial Sequence: Synthetic oligonucleotide"
  source 1..42: mol_type = other DNA
gagggtagcaatatatgtaaatgctgatgcaagaggcgcaga

SEQ ID NO: 111 - DNA, 42 bp, synthetic construct
  misc_feature 1..42: note = source = /note="Description of Artificial Sequence: Synthetic oligonucleotide"
  source 1..42: mol_type = other DNA
cggtcaatcataacatcaagaaaacaaaattagcatcggaac

SEQ ID NO: 112 - DNA, 40 bp, synthetic construct
  misc_feature 1..40: note = source = /note="Description of Artificial Sequence: Synthetic oligonucleotide"
  source 1..40: mol_type = other DNA
gattcgcctcatttcgcaaatggtcaataattacatcggg

SEQ ID NO: 113 - DNA, 42 bp, synthetic construct
  misc_feature 1..42: note = source = /note="Description of Artificial Sequence: Synthetic oligonucleotide"
  source 1..42: mol_type = other DNA
aataatggaagcaccctcagcagcgaaagacaattacattta

SEQ ID NO: 114 - DNA, 42 bp, synthetic construct
  misc_feature 1..42: note = source = /note="Description of Artificial Sequence: Synthetic oligonucleotide"
  source 1..42: mol_type = other DNA
tgcgggatcgtggttagaacctaccatatcaatttgaaagag

SEQ ID NO: 115 - DNA, 42 bp, synthetic construct
  misc_feature 1..42: note = source = /note="Description of Artificial Sequence: Synthetic oligonucleotide"
  source 1..42: mol_type = other DNA
gacagatgaacactaacaactaatagattagaaggccgcttt

SEQ ID NO: 116 - DNA, 40 bp, synthetic construct
  misc_feature 1..40: note = source = /note="Description of Artificial Sequence: Synthetic oligonucleotide"
  source 1..40: mol_type = other DNA
ctcaaatatttggggcgcgagctgaaaaggtctaaagcat

SEQ ID NO: 117 - DNA, 42 bp, synthetic construct
  misc_feature 1..42: note = source = /note="Description of Artificial Sequence: Synthetic oligonucleotide"
  source 1..42: mol_type = other DNA
catcgccattactgaggcttgcagggagttaagccgtcaata

SEQ ID NO: 118 - DNA, 42 bp, synthetic construct
  misc_feature 1..42: note = source = /note="Description of Artificial Sequence: Synthetic oligonucleotide"
  source 1..42: mol_type = other DNA
atattcggtcgaaaataccgaacgaaccaccaggctggctga

SEQ ID NO: 119 - DNA, 42 bp, synthetic construct
  misc_feature 1..42: note = source = /note="Description of Artificial Sequence: Synthetic oligonucleotide"
  source 1..42: mol_type = other DNA
ccttcatcaagtatccagaacaatattaccgccataaccgat

SEQ ID NO: 120 - DNA, 42 bp, synthetic construct
  misc_feature 1..42: note = source = /note="Description of Artificial Sequence: Synthetic oligonucleotide"
  source 1..42: mol_type = other DNA
tctttaggagcggtgtacagaccaggcgcatagcagaagata

SEQ ID NO: 121 - DNA, 42 bp, synthetic construct
  misc_feature 1..42: note = source = /note="Description of Artificial Sequence: Synthetic oligonucleotide"
  source 1..42: mol_type = other DNA
aaacagaggtggctcattcagtgaataaggctatctaaaata

SEQ ID NO: 122 - DNA, 42 bp, synthetic construct
  misc_feature 1..42: note = source = /note="Description of Artificial Sequence: Synthetic oligonucleotide"
  source 1..42: mol_type = other DNA
gtaacaaagctaggcggtcagtattaacaccgtgcggaatcg

SEQ ID NO: 123 - DNA, 42 bp, synthetic construct
  misc_feature 1..42: note = source = /note="Description of Artificial Sequence: Synthetic oligonucleotide"
  source 1..42: mol_type = other DNA
tcataaatattttgcctgagtagaagaactcaccaaatcaac

SEQ ID NO: 124 - DNA, 42 bp, synthetic construct
  misc_feature 1..42: note = source = /note="Description of Artificial Sequence: Synthetic oligonucleotide"
  source 1..42: mol_type = other DNA
aaatcaacagtagactggatagcgtccaataccctgcaacag

SEQ ID NO: 125 - DNA, 42 bp, synthetic construct
  misc_feature 1..42: note = source = /note="Description of Artificial Sequence: Synthetic oligonucleotide"
  source 1..42: mol_type = other DNA
tgccacgctgaaatcaaaaatcaggtctttacgtcagttggc

SEQ ID NO: 126 - DNA, 42 bp, synthetic construct
  misc_feature 1..42: note = source = /note="Description of Artificial Sequence: Synthetic oligonucleotide"
  source 1..42: mol_type = other DNA
gaatgaccatagagccagcagcaaatgaaaaatggcatcaat

SEQ ID NO: 127 - DNA, 42 bp, synthetic construct
  misc_feature 1..42: note = source = /note="Description of Artificial Sequence: Synthetic oligonucleotide"
  source 1..42: mol_type = other DNA
tctactaatagtaaccgttgtagcaatacttccagaaaacga

SEQ ID NO: 128 - DNA, 42 bp, synthetic construct
  misc_feature 1..42: note = source = /note="Description of Artificial Sequence: Synthetic oligonucleotide"
  source 1..42: mol_type = other DNA
tatattttcatcaaaccctcaatcaatatctgcctgactatt

SEQ ID NO: 129 - DNA, 42 bp, synthetic construct
  misc_feature 1..42: note = source = /note="Description of Artificial Sequence: Synthetic oligonucleotide"
  source 1..42: mol_type = other DNA
atagtcagaagaatatacagtaacagtaccttcctgtttagc

SEQ ID NO: 130 - DNA, 42 bp, synthetic construct
  misc_feature 1..42: note = source = /note="Description of Artificial Sequence: Synthetic oligonucleotide"
  source 1..42: mol_type = other DNA
gtaaaatgttttgaaaggaattgaggaaggtttgccctgacg

SEQ ID NO: 131 - DNA, 42 bp, synthetic construct
  misc_feature 1..42: note = source = /note="Description of Artificial Sequence: Synthetic oligonucleotide"
  source 1..42: mol_type = other DNA
agaaacaccagaaataaagaaattgcgtagatgggggtaata

SEQ ID NO: 132 - DNA, 42 bp, synthetic construct
  misc_feature 1..42: note = source = /note="Description of Artificial Sequence: Synthetic oligonucleotide"
  source 1..42: mol_type = other DNA
atgatgaaacaaagggaaccgaactgaccaacaattatttgc

SEQ ID NO: 133 - DNA, 42 bp, synthetic construct
  misc_feature 1..42: note = source = /note="Description of Artificial Sequence: Synthetic oligonucleotide"
  source 1..42: mol_type = other DNA
acgtaaaacagaacgagtagtaaattgggcttgcaaaagaag

SEQ ID NO: 134 - DNA, 42 bp, synthetic construct
  misc_feature 1..42: note = source = /note="Description of Artificial Sequence: Synthetic oligonucleotide"
  source 1..42: mol_type = other DNA
gcagaggcgaatgcaaaagaagttttgccagatttcaggttt

SEQ ID NO: 135 - DNA, 42 bp, synthetic construct
  misc_feature 1..42: note = source = /note="Description of Artificial Sequence: Synthetic oligonucleotide"
  source 1..42: mol_type = other DNA
aacgtcagatgcaaagcggattgcatcaaaaaacaaaatcgc

SEQ ID NO: 136 - DNA, 42 bp, synthetic construct
  misc_feature 1..42: note = source = /note="Description of Artificial Sequence: Synthetic oligonucleotide"
  source 1..42: mol_type = other DNA
cctcccgacttgcgggaggttctgcattaatgaatcggccaa

SEQ ID NO: 137 - DNA, 42 bp, synthetic construct
  misc_feature 1..42: note = source = /note="Description of Artificial Sequence: Synthetic oligonucleotide"
  source 1..42: mol_type = other DNA
taactcacattaattgcgttgagaattaactgaacaccctga

SEQ ID NO: 138 - DNA, 42 bp, synthetic construct
  misc_feature 1..42: note = source = /note="Description of Artificial Sequence: Synthetic oligonucleotide"
  source 1..42: mol_type = other DNA
aaaatgaaaatagcagcctttttaaatttttgttaaatcagc

SEQ ID NO: 139 - DNA, 42 bp, synthetic construct
  misc_feature 1..42: note = source = /note="Description of Artificial Sequence: Synthetic oligonucleotide"
  source 1..42: mol_type = other DNA
aacaggaagattgtataagcatacaattttatcctgaatctt

SEQ ID NO: 140 - DNA, 42 bp, synthetic construct
  misc_feature 1..42: note = source = /note="Description of Artificial Sequence: Synthetic oligonucleotide"
  source 1..42: mol_type = other DNA
agttgctattttgcacccagcaatatttaaattgtaaacgtt

SEQ ID NO: 141 - DNA, 42 bp, synthetic construct
  misc_feature 1..42: note = source = /note="Description of Artificial Sequence: Synthetic oligonucleotide"
  source 1..42: mol_type = other DNA
aatattttgttaaaattcgcaacagagagaataacataaaaa

SEQ ID NO: 142 - DNA, 42 bp, synthetic construct
  misc_feature 1..42: note = source = /note="Description of Artificial Sequence: Synthetic oligonucleotide"
  source 1..42: mol_type = other DNA
cagggaagcgcattagacgggcgctcactgcccgctttccag

SEQ ID NO: 143 - DNA, 42 bp, synthetic construct
  misc_feature 1..42: note = source = /note="Description of Artificial Sequence: Synthetic oligonucleotide"
  source 1..42: mol_type = other DNA
tcgggaaacctgtcgtgccagttgaagccttaaatcaagatt

SEQ ID NO: 144 - DNA, 42 bp, synthetic construct
  misc_feature 1..42: note = source = /note="Description of Artificial Sequence: Synthetic oligonucleotide"
  source 1..42: mol_type = other DNA
accaacgctaacgagcgtctttgtcaatcatatgtaccccgg

SEQ ID NO: 145 - DNA, 42 bp, synthetic construct
  misc_feature 1..42: note = source = /note="Description of Artificial Sequence: Synthetic oligonucleotide"
  source 1..42: mol_type = other DNA
ggtcattgcctgagagtctggacgattttttgtttaacgtca

SEQ ID NO: 146 - DNA, 42 bp, synthetic construct
  misc_feature 1..42: note = source = /note="Description of Artificial Sequence: Synthetic oligonucleotide"
  source 1..42: mol_type = other DNA
ttatcccaatccaaataagaaagcaaacaagagaatcgatga

SEQ ID NO: 147 - DNA, 42 bp, synthetic construct
  misc_feature 1..42: note = source = /note="Description of Artificial Sequence: Synthetic oligonucleotide"
  source 1..42: mol_type = other DNA
acggtaatcgtaaaactagcatccagagcctaatttgccagt

SEQ ID NO: 148 - DNA, 42 bp, synthetic construct
  misc_feature 1..42: note = source = /note="Description of Artificial Sequence: Synthetic oligonucleotide"
  source 1..42: mol_type = other DNA
ccgccaccctcagagccaccatttcatcaacattaaatgtga

SEQ ID NO: 149 - DNA, 42 bp, synthetic construct
  misc_feature 1..42: note = source = /note="Description of Artificial Sequence: Synthetic oligonucleotide"
  source 1..42: mol_type = other DNA
tcattttttaaccaataggaagtagcgcgttttcatcggcat

SEQ ID NO: 150 - DNA, 42 bp, synthetic construct
  misc_feature 1..42: note = source = /note="Description of Artificial Sequence: Synthetic oligonucleotide"
  source 1..42: mol_type = other DNA
aaccatcgatagcagcaccgttggggtgcctaatgagtgagc

SEQ ID NO: 151 - DNA, 42 bp, synthetic construct
  misc_feature 1..42: note = source = /note="Description of Artificial Sequence: Synthetic oligonucleotide"
  source 1..42: mol_type = other DNA
agcttgcatgcctgcaggtcgtagttgcgccgacaatgacaa

SEQ ID NO: 152 - DNA, 42 bp, synthetic construct
  misc_feature 1..42: note = source = /note="Description of Artificial Sequence: Synthetic oligonucleotide"
  source 1..42: mol_type = other DNA
tttcggtcatagcccccttatagagatctacaaaggctatca

SEQ ID NO: 153 - DNA, 42 bp, synthetic construct
  misc_feature 1..42: note = source = /note="Description of Artificial Sequence: Synthetic oligonucleotide"
  source 1..42: mol_type = other DNA
cctcatatattttaaatgcaaaaaaaaggctccaaaaggagc

SEQ ID NO: 154 - DNA, 42 bp, synthetic construct
  misc_feature 1..42: note = source = /note="Description of Artificial Sequence: Synthetic oligonucleotide"
  source 1..42: mol_type = other DNA
tttcacgttgaaaatctccaatgcctgagtaatgtgtaggta

SEQ ID NO: 155 - DNA, 42 bp, synthetic construct
  misc_feature 1..42: note = source = /note="Description of Artificial Sequence: Synthetic oligonucleotide"
  source 1..42: mol_type = other DNA
aagattcaaaagggtgagaaatgagaatagaaaggaacaact

SEQ ID NO: 156 - DNA, 42 bp, synthetic construct
  misc_feature 1..42: note = source = /note="Description of Artificial Sequence: Synthetic oligonucleotide"
  source 1..42: mol_type = other DNA
tcatagttagcgtaacgatcttggtcatagctgtttcctgtg

SEQ ID NO: 157 - DNA, 42 bp, synthetic construct
  misc_feature 1..42: note = source = /note="Description of Artificial Sequence: Synthetic oligonucleotide"
  source 1..42: mol_type = other DNA
ccgagctcgaattcgtaatcaaaagttttgtcgtctttccag

SEQ ID NO: 158 - DNA, 42 bp, synthetic construct
  misc_feature 1..42: note = source = /note="Description of Artificial Sequence: Synthetic oligonucleotide"
  source 1..42: mol_type = other DNA
acgttagtaaatgaattttcttctccgtgggaacaaacggcg

SEQ ID NO: 159 - DNA, 42 bp, synthetic construct
  misc_feature 1..42: note = source = /note="Description of Artificial Sequence: Synthetic oligonucleotide"
  source 1..42: mol_type = other DNA
gcgagtaacaacccgtcggatgtatgggattttgctaaacaa

SEQ ID NO: 160 - DNA, 42 bp, synthetic construct
  misc_feature 1..42: note = source = /note="Description of Artificial Sequence: Synthetic oligonucleotide"
  source 1..42: mol_type = other DNA
ctttaattgtatcggtttatctcacgttggtgtagatgggcg

SEQ ID NO: 161 - DNA, 42 bp, synthetic construct
  misc_feature 1..42: note = source = /note="Description of Artificial Sequence: Synthetic oligonucleotide"
  source 1..42: mol_type = other DNA
gattgaccgtaatgggataggagcttgctttcgaggtgaatt

SEQ ID NO: 162 - DNA, 42 bp, synthetic construct
  misc_feature 1..42: note = source = /note="Description of Artificial Sequence: Synthetic oligonucleotide"
  source 1..42: mol_type = other DNA
ctttcaacagtttcagcggagggccggagacagtcaaatcac

SEQ ID NO: 163 - DNA, 42 bp, synthetic construct
  misc_feature 1..42: note = source = /note="Description of Artificial Sequence: Synthetic oligonucleotide"
  source 1..42: mol_type = other DNA
catcaatatgatattcaaccgtcagagccgccaccctcagaa

SEQ ID NO: 164 - DNA, 42 bp, synthetic construct
  misc_feature 1..42: note = source = /note="Description of Artificial Sequence: Synthetic oligonucleotide"
  source 1..42: mol_type = other DNA
ccaccaccggaaccgcctcccttctagctgataaattaatgc

SEQ ID NO: 165 - DNA, 42 bp, synthetic construct
  misc_feature 1..42: note = source = /note="Description of Artificial Sequence: Synthetic oligonucleotide"
  source 1..42: mol_type = other DNA
tgaaattgttatccgctcacagcattgacaggaggttgaggc

SEQ ID NO: 166 - DNA, 42 bp, synthetic construct
  misc_feature 1..42: note = source = /note="Description of Artificial Sequence: Synthetic oligonucleotide"
  source 1..42: mol_type = other DNA
ccaccaccagagccgccgccaattccacacaacatacgagcc

SEQ ID NO: 167 - DNA, 42 bp, synthetic construct
  misc_feature 1..42: note = source = /note="Description of Artificial Sequence: Synthetic oligonucleotide"
  source 1..42: mol_type = other DNA
tctggccttcctgtagccagcccctcagagccgccaccagaa

SEQ ID NO: 168 - DNA, 42 bp, synthetic construct
  misc_feature 1..42: note = source = /note="Description of Artificial Sequence: Synthetic oligonucleotide"
  source 1..42: mol_type = other DNA
cggagagggtagctatttttgtagcgtttgccatcttttcat

SEQ ID NO: 169 - DNA, 42 bp, synthetic construct
  misc_feature 1..42: note = source = /note="Description of Artificial Sequence: Synthetic oligonucleotide"
  source 1..42: mol_type = other DNA
tcttaaacagcttgataccgaactctagaggatccccgggta

SEQ ID NO: 170 - DNA, 42 bp, synthetic construct
  misc_feature 1..42: note = source = /note="Description of Artificial Sequence: Synthetic oligonucleotide"
  source 1..42: mol_type = other DNA
ggaagcataaagtgtaaagccaatcagtagcgacagaatcaa

SEQ ID NO: 171 - DNA, 42 bp, synthetic construct
  misc_feature 1..42: note = source = /note="Description of Artificial Sequence: Synthetic oligonucleotide"
  source 1..42: mol_type = other DNA
gtttgcctttagcgtcagactcgccatcaaaaataattcgcg

SEQ ID NO: 172 - DNA, 42 bp, synthetic construct
  misc_feature 1..42: note = source = /note="Description of Artificial Sequence: Synthetic oligonucleotide"
  source 1..42: mol_type = other DNA
acaggtagaaagattcatcagactccagccagctttccggca

SEQ ID NO: 173 - DNA, 42 bp, synthetic construct
  misc_feature 1..42: note = source = /note="Description of Artificial Sequence: Synthetic oligonucleotide"
  source 1..42: mol_type = other DNA
catcgtaaccgtgcatctgcctggtttaatttcaactttaat

SEQ ID NO: 174 - DNA, 42 bp, synthetic construct
  misc_feature 1..42: note = source = /note="Description of Artificial Sequence: Synthetic oligonucleotide"
  source 1..42: mol_type = other DNA
attcagtgaataaggcttgccgtaaaacgacggccagtgcca

SEQ ID NO: 175 - DNA, 42 bp, synthetic construct
  misc_feature 1..42: note = source = /note="Description of Artificial Sequence: Synthetic oligonucleotide"
  source 1..42: mol_type = other DNA
cattgtgaattaccttatgcgaaggataaaaatttttagaac

SEQ ID NO: 176 - DNA, 42 bp, synthetic construct
  misc_feature 1..42: note = source = /note="Description of Artificial Sequence: Synthetic oligonucleotide"
  source 1..42: mol_type = other DNA
tagcaaaattaagcaataaagtctactaatagtagtagcatt

SEQ ID NO: 177 - DNA, 42 bp, synthetic construct
  misc_feature 1..42: note = source = /note="Description of Artificial Sequence: Synthetic oligonucleotide"
  source 1..42: mol_type = other DNA
cgaacgagtagatttagtttgcgctattacgccagctggcga

SEQ ID NO: 178 - DNA, 42 bp, synthetic construct
  misc_feature 1..42: note = source = /note="Description of Artificial Sequence: Synthetic oligonucleotide"
  source 1..42: mol_type = other DNA
ggcgatcggtgcgggcctcttaccattagatacatttcgcaa

SEQ ID NO: 179 - DNA, 42 bp, synthetic construct
  misc_feature 1..42: note = source = /note="Description of Artificial Sequence: Synthetic oligonucleotide"
  source 1..42: mol_type = other DNA
atggtcaataacctgtttagcaggcaaagcgccattcgccat

SEQ ID NO: 180 - DNA, 42 bp, synthetic construct
  misc_feature 1..42: note = source = /note="Description of Artificial Sequence: Synthetic oligonucleotide"
  source 1..42: mol_type = other DNA
ccgcttctggtgccggaaacctatattttcatttggggcgcg

SEQ ID NO: 181 - DNA, 42 bp, synthetic construct
  misc_feature 1..42: note = source = /note="Description of Artificial Sequence: Synthetic oligonucleotide"
  source 1..42: mol_type = other DNA
agctgaaaaggtggcatcaatcctcagagcataaagctaaat

SEQ ID NO: 182 - DNA, 42 bp, synthetic construct
  misc_feature 1..42: note = source = /note="Description of Artificial Sequence: Synthetic oligonucleotide"
  source 1..42: mol_type = other DNA
cggttgtaccaaaaacattataactaacggaacaacattatt

SEQ ID NO: 183 - DNA, 42 bp, synthetic construct
  misc_feature 1..42: note = source = /note="Description of Artificial Sequence: Synthetic oligonucleotide"
  source 1..42: mol_type = other DNA
aaaatctacgttaataaaacggaccctgtaatacttttgcgg

SEQ ID NO: 184 - DNA, 42 bp, synthetic construct
  misc_feature 1..42: note = source = /note="Description of Artificial Sequence: Synthetic oligonucleotide"
  source 1..42: mol_type = other DNA
aagggggatgtgctgcaaggcacgccaaaaggaattacgagg

SEQ ID NO: 185 - DNA, 42 bp, synthetic construct
  misc_feature 1..42: note = source = /note="Description of Artificial Sequence: Synthetic oligonucleotide"
  source 1..42: mol_type = other DNA
ttcaactaatgcagatacatagattaagttgggtaacgccag

SEQ ID NO: 186 - DNA, 42 bp, synthetic construct
  misc_feature 1..42: note = source = /note="Description of Artificial Sequence: Synthetic oligonucleotide"
  source 1..42: mol_type = other DNA
tatcggcctcaggaagatcgcttgagatttaggaataccaca

SEQ ID NO: 187 - DNA, 42 bp, synthetic construct
  misc_feature 1..42: note = source = /note="Description of Artificial Sequence: Synthetic oligonucleotide"
  source 1..42: mol_type = other DNA
gagaagcctttatttcaacgcattttaagaactggctcatta

SEQ ID NO: 188 - DNA, 42 bp, synthetic construct
  misc_feature 1..42: note = source = /note="Description of Artificial Sequence: Synthetic oligonucleotide"
  source 1..42: mol_type = other DNA
ggttttcccagtcacgacgttctgacgagaaacaccagaacg

SEQ ID NO: 189 - DNA, 42 bp, synthetic construct
  misc_feature 1..42: note = source = /note="Description of Artificial Sequence: Synthetic oligonucleotide"
  source 1..42: mol_type = other DNA
agtagtaaattgggcttgagaagtttgaggggacgacgacag

SEQ ID NO: 190 - DNA, 42 bp, synthetic construct
  misc_feature 1..42: note = source = /note="Description of Artificial Sequence: Synthetic oligonucleotide"
  source 1..42: mol_type = other DNA
tctttccttatcattccaagacgtaaaacagaaataaagaaa

SEQ ID NO: 191 - DNA, 42 bp, synthetic construct
  misc_feature 1..42: note = source = /note="Description of Artificial Sequence: Synthetic oligonucleotide"
  source 1..42: mol_type = other DNA
ttgtttggattatacttctgaaaagttaccagaaggaaaccg

SEQ ID NO: 192 - DNA, 42 bp, synthetic construct
  misc_feature 1..42: note = source = /note="Description of Artificial Sequence: Synthetic oligonucleotide"
  source 1..42: mol_type = other DNA
aatgaaatagcaatagctatcaatggattatttacattggca

SEQ ID NO: 193 - DNA, 42 bp, synthetic construct
  misc_feature 1..42: note = source = /note="Description of Artificial Sequence: Synthetic oligonucleotide"
  source 1..42: mol_type = other DNA
ccagccattgcaacaggaaaagccgtttttattttcatcgta

SEQ ID NO: 194 - DNA, 42 bp, synthetic construct
  misc_feature 1..42: note = source = /note="Description of Artificial Sequence: Synthetic oligonucleotide"
  source 1..42: mol_type = other DNA
gcactcatcgagaacaagcaaacgctcatggaaatacctaca

SEQ ID NO: 195 - DNA, 42 bp, synthetic construct
  misc_feature 1..42: note = source = /note="Description of Artificial Sequence: Synthetic oligonucleotide"
  source 1..42: mol_type = other DNA
ttttgacgctcaatcgtctgattaccgaagccctttttaaga

SEQ ID NO: 196 - DNA, 42 bp, synthetic construct
  misc_feature 1..42: note = source = /note="Description of Artificial Sequence: Synthetic oligonucleotide"
  source 1..42: mol_type = other DNA
aaagtaagcagatagccgaacataatggaagggttagaacct

SEQ ID NO: 197 - DNA, 42 bp, synthetic construct
  misc_feature 1..42: note = source = /note="Description of Artificial Sequence: Synthetic oligonucleotide"
  source 1..42: mol_type = other DNA
accatatcaaaattatttgcaacgggtattaaaccaagtacc

SEQ ID NO: 198 - DNA, 42 bp, synthetic construct
  misc_feature 1..42: note = source = /note="Description of Artificial Sequence: Synthetic oligonucleotide"
  source 1..42: mol_type = other DNA
ggaatcattaccgcgcccaattcaaactatcggccttgctgg

SEQ ID NO: 199 - DNA, 42 bp, synthetic construct
  misc_feature 1..42: note = source = /note="Description of Artificial Sequence: Synthetic oligonucleotide"
  source 1..42: mol_type = other DNA
aattaaccgttgtagcaatacccaataataagagcaagaaac

SEQ ID NO: 200 - DNA, 42 bp, synthetic construct
  misc_feature 1..42: note = source = /note="Description of Artificial Sequence: Synthetic oligonucleotide"
  source 1..42: mol_type = other DNA
acaaagtcagagggtaattgaccgcctggccctgagagagtt

SEQ ID NO: 201 - DNA, 42 bp, synthetic construct
  misc_feature 1..42: note = source = /note="Description of Artificial Sequence: Synthetic oligonucleotide"
  source 1..42: mol_type = other DNA
tattgggcgccagggtggtttaacgcgaggcgttttagcgaa

SEQ ID NO: 202 - DNA, 42 bp, synthetic construct
  misc_feature 1..42: note = source = /note="Description of Artificial Sequence: Synthetic oligonucleotide"
  source 1..42: mol_type = other DNA
aggcttatccggtattctaagttcttttcaccagtgagacgg

SEQ ID NO: 203 - DNA, 42 bp, synthetic construct
  misc_feature 1..42: note = source = /note="Description of Artificial Sequence: Synthetic oligonucleotide"
  source 1..42: mol_type = other DNA
gcaacagctgattgcccttcagcgctaatatcagagagataa

SEQ ID NO: 204 - DNA, 42 bp, synthetic construct
  misc_feature 1..42: note = source = /note="Description of Artificial Sequence: Synthetic oligonucleotide"
  source 1..42: mol_type = other DNA
cccacaagaattgagttaagcttctttgattagtaataacat

SEQ ID NO: 205 - DNA, 42 bp, synthetic construct
  misc_feature 1..42: note = source = /note="Description of Artificial Sequence: Synthetic oligonucleotide"
  source 1..42: mol_type = other DNA
cacttgcctgagtagaagaacagcaagcaaatcagatataga

SEQ ID NO: 206 - DNA, 42 bp, synthetic construct
  misc_feature 1..42: note = source = /note="Description of Artificial Sequence: Synthetic oligonucleotide"
  source 1..42: mol_type = other DNA
ttccagtaagcgtcatacatgtgacctgaaagcgtaagaata

SEQ ID NO: 207 - DNA, 42 bp, synthetic construct
  misc_feature 1..42: note = source = /note="Description of Artificial Sequence: Synthetic oligonucleotide"
  source 1..42: mol_type = other DNA
gattcaccagtcacacgaccaaaggtgaattatcaccgtcac

SEQ ID NO: 208 - DNA, 42 bp, synthetic construct
  misc_feature 1..42: note = source = /note="Description of Artificial Sequence: Synthetic oligonucleotide"
  source 1..42: mol_type = other DNA
caaaagggcgacattcaaccgaattcatcaatataatcctga

SEQ ID NO: 209 - DNA, 42 bp, synthetic construct
  misc_feature 1..42: note = source = /note="Description of Artificial Sequence: Synthetic oligonucleotide"
  source 1..42: mol_type = other DNA
tttacaaacaattcgacaactactttttcatgaggaagtttc

SEQ ID NO: 210 - DNA, 42 bp, synthetic construct
  misc_feature 1..42: note = source = /note="Description of Artificial Sequence: Synthetic oligonucleotide"
  source 1..42: mol_type = other DNA
caaccatcgcccacgcataacaaagaacgtggactccaacgt

SEQ ID NO: 211 - DNA, 42 bp, synthetic construct
  misc_feature 1..42: note = source = /note="Description of Artificial Sequence: Synthetic oligonucleotide"
  source 1..42: mol_type = other DNA
gcagcaagcggtccacgctggggccggaaacgtcaccaatga

SEQ ID NO: 212 - DNA, 42 bp, synthetic construct
  misc_feature 1..42: note = source = /note="Description of Artificial Sequence: Synthetic oligonucleotide"
  source 1..42: mol_type = other DNA
cgacttgagccatttgggaataaagagtctgtccatcacgca

SEQ ID NO: 213 - DNA, 42 bp, synthetic construct
  misc_feature 1..42: note = source = /note="Description of Artificial Sequence: Synthetic oligonucleotide"
  source 1..42: mol_type = other DNA
cttgcagggagttaaaggccgataacgtgctttcctcgttag

SEQ ID NO: 214 - DNA, 42 bp, synthetic construct
  misc_feature 1..42: note = source = /note="Description of Artificial Sequence: Synthetic oligonucleotide"
  source 1..42: mol_type = other DNA
aatcagagcgggagctaaacaccgtaacactgagtttcgtca

SEQ ID NO: 215 - DNA, 42 bp, synthetic construct
  misc_feature 1..42: note = source = /note="Description of Artificial Sequence: Synthetic oligonucleotide"
  source 1..42: mol_type = other DNA
ggaggtttagtaccgccaccctgagtaacattatcattttgc

SEQ ID NO: 216 - DNA, 42 bp, synthetic construct
  misc_feature 1..42: note = source = /note="Description of Artificial Sequence: Synthetic oligonucleotide"
  source 1..42: mol_type = other DNA
acgttattaattttaaaagtttcagaaccgccaccctcagaa

SEQ ID NO: 217 - DNA, 42 bp, synthetic construct
  misc_feature 1..42: note = source = /note="Description of Artificial Sequence: Synthetic oligonucleotide"
  source 1..42: mol_type = other DNA
ccgccaccctcagagccaccagaatggctattagtctttaat

SEQ ID NO: 218 - DNA, 42 bp, synthetic construct
  misc_feature 1..42: note = source = /note="Description of Artificial Sequence: Synthetic oligonucleotide"
  source 1..42: mol_type = other DNA
cgtggcacagacaatatttttccctcattttcagggatagca

SEQ ID NO: 219 - DNA, 42 bp, synthetic construct
  misc_feature 1..42: note = source = /note="Description of Artificial Sequence: Synthetic oligonucleotide"
  source 1..42: mol_type = other DNA
cagcagcgaaagacagcatcgacatcgccattaaaaataccg

SEQ ID NO: 220 - DNA, 42 bp, synthetic construct
  misc_feature 1..42: note = source = /note="Description of Artificial Sequence: Synthetic oligonucleotide"
  source 1..42: mol_type = other DNA
gcgcgaactgatagccctaaagaacgagggtagcaacggcta

SEQ ID NO: 221 - DNA, 42 bp, synthetic construct
  misc_feature 1..42: note = source = /note="Description of Artificial Sequence: Synthetic oligonucleotide"
  source 1..42: mol_type = other DNA
agcccaataggaacccatgtaggaggccgattaaagggattt

SEQ ID NO: 222 - DNA, 42 bp, synthetic construct
  misc_feature 1..42: note = source = /note="Description of Artificial Sequence: Synthetic oligonucleotide"
  source 1..42: mol_type = other DNA
atcaaaagaatagcccgagatgtagcattccacagacagccc

SEQ ID NO: 223 - DNA, 42 bp, synthetic construct
  misc_feature 1..42: note = source = /note="Description of Artificial Sequence: Synthetic oligonucleotide"
  source 1..42: mol_type = other DNA
ccagtacaaactacaacgcctagggttgagtgttgttccagt

SEQ ID NO: 224 - DNA, 42 bp, synthetic construct
  misc_feature 1..42: note = source = /note="Description of Artificial Sequence: Synthetic oligonucleotide"
  source 1..42: mol_type = other DNA
tagacaggaacggtacgccaggcgcagtctctgaatttaccg

SEQ ID NO: 225 - DNA, 42 bp, synthetic construct
  misc_feature 1..42: note = source = /note="Description of Artificial Sequence: Synthetic oligonucleotide"
  source 1..42: mol_type = other DNA
aggtcagacgattggccttgaaatcggcaaaatcccttataa

SEQ ID NO: 226 - DNA, 42 bp, synthetic construct
  misc_feature 1..42: note = source = /note="Description of Artificial Sequence: Synthetic oligonucleotide"
  source 1..42: mol_type = other DNA
cctgtttgatggtggttccgatattcacaaacaaataaatcc

SEQ ID NO: 227 - DNA, 42 bp, synthetic construct
  misc_feature 1..42: note = source = /note="Description of Artificial Sequence: Synthetic oligonucleotide"
  source 1..42: mol_type = other DNA
tcattaaagccagaatggaaaaatcctgagaagtgtttttat

SEQ ID NO: 228 - DNA, 42 bp, synthetic construct
  misc_feature 1..42: note = source = /note="Description of Artificial Sequence: Synthetic oligonucleotide"
  source 1..42: mol_type = other DNA
ggaacaaagaaaccaccagaagggtcagtgccttgagtaaca

SEQ ID NO: 229 - DNA, 42 bp, synthetic construct
  misc_feature 1..42: note = source = /note="Description of Artificial Sequence: Synthetic oligonucleotide"
  source 1..42: mol_type = other DNA
tactggtaataagttttaacgggagcggaattatcatcatat

SEQ ID NO: 230 - DNA, 42 bp, synthetic construct
  misc_feature 1..42: note = source = /note="Description of Artificial Sequence: Synthetic oligonucleotide"
  source 1..42: mol_type = other DNA
ccaacagagatagaacccttcgcttttgatgatacaggagtg

SEQ ID NO: 231 - DNA, 42 bp, synthetic construct
  misc_feature 1..42: note = source = /note="Description of Artificial Sequence: Synthetic oligonucleotide"
  source 1..42: mol_type = other DNA
aatcagtgaggccaccgagtatagagccagcaaaatcaccag

SEQ ID NO: 232 - DNA, 42 bp, synthetic construct
  misc_feature 1..42: note = source = /note="Description of Artificial Sequence: Synthetic oligonucleotide"
  source 1..42: mol_type = other DNA
tagcaccattaccattagcaatttgccccagcaggcgaaaat

SEQ ID NO: 233 - DNA, 42 bp, synthetic construct
  misc_feature 1..42: note = source = /note="Description of Artificial Sequence: Synthetic oligonucleotide"
  source 1..42: mol_type = other DNA
ttggaacaagagtccactattcgatatattcggtcgctgagg

SEQ ID NO: 234 - DNA, 42 bp, synthetic construct
  misc_feature 1..42: note = source = /note="Description of Artificial Sequence: Synthetic oligonucleotide"
  source 1..42: mol_type = other DNA
cagaggctttgaggactaaagcgtattaaatcctttgcccga

SEQ ID NO: 235 - DNA, 42 bp, synthetic construct
  misc_feature 1..42: note = source = /note="Description of Artificial Sequence: Synthetic oligonucleotide"
  source 1..42: mol_type = other DNA
tcctgattatcagatgatggcattgagggagggaaggtaaat

SEQ ID NO: 236 - DNA, 42 bp, synthetic construct
  misc_feature 1..42: note = source = /note="Description of Artificial Sequence: Synthetic oligonucleotide"
  source 1..42: mol_type = other DNA
attgacggaaattattcattagtaataaaagggacattctgg

SEQ ID NO: 237 - DNA, 42 bp, synthetic construct
  misc_feature 1..42: note = source = /note="Description of Artificial Sequence: Synthetic oligonucleotide"
  source 1..42: mol_type = other DNA
tttgccagagggggtaatagtgtgccacgctgagagccagca

SEQ ID NO: 238 - DNA, 42 bp, synthetic construct
  misc_feature 1..42: note = source = /note="Description of Artificial Sequence: Synthetic oligonucleotide"
  source 1..42: mol_type = other DNA
aacgaaccaccagcagaagatatgaacggtgtacagaccagg

SEQ ID NO: 239 - DNA, 42 bp, synthetic construct
  misc_feature 1..42: note = source = /note="Description of Artificial Sequence: Synthetic oligonucleotide"
  source 1..42: mol_type = other DNA
cggaacgaggcgcagacggtcgaggatttagaagtattagac

SEQ ID NO: 240 - DNA, 42 bp, synthetic construct
  misc_feature 1..42: note = source = /note="Description of Artificial Sequence: Synthetic oligonucleotide"
  source 1..42: mol_type = other DNA
caaagggcgaaaaaccgtctaatcaacgtaacaaagctgctc

SEQ ID NO: 241 - DNA, 42 bp, synthetic construct
  misc_feature 1..42: note = source = /note="Description of Artificial Sequence: Synthetic oligonucleotide"
  source 1..42: mol_type = other DNA
cgcataggctggctgaccttcgccgctacagggcgcgtacta

SEQ ID NO: 242 - DNA, 42 bp, synthetic construct
  misc_feature 1..42: note = source = /note="Description of Artificial Sequence: Synthetic oligonucleotide"
  source 1..42: mol_type = other DNA
cgtggcgagaaaggaagggaaatatgcaactaaagtacggtg

SEQ ID NO: 243 - DNA, 42 bp, synthetic construct
  misc_feature 1..42: note = source = /note="Description of Artificial Sequence: Synthetic oligonucleotide"
  source 1..42: mol_type = other DNA
aggattagagagtacctttaagaaaggaattgaggaaggtta

SEQ ID NO: 244 - DNA, 42 bp, synthetic construct
  misc_feature 1..42: note = source = /note="Description of Artificial Sequence: Synthetic oligonucleotide"
  source 1..42: mol_type = other DNA
tcagttggcaaatcaacagttttgctccttttgataagaggt

SEQ ID NO: 245 - DNA, 42 bp, synthetic construct
  misc_feature 1..42: note = source = /note="Description of Artificial Sequence: Synthetic oligonucleotide"
  source 1..42: mol_type = other DNA
catttttgcggatggcttagatcaccttgctgaacctcaaat

SEQ ID NO: 246 - DNA, 42 bp, synthetic construct
  misc_feature 1..42: note = source = /note="Description of Artificial Sequence: Synthetic oligonucleotide"
  source 1..42: mol_type = other DNA
gcaaatgaaaaatctaaagcagcttaattgctgaatataatg

SEQ ID NO: 247 - DNA, 42 bp, synthetic construct
  misc_feature 1..42: note = source = /note="Description of Artificial Sequence: Synthetic oligonucleotide"
  source 1..42: mol_type = other DNA
ctgtagctcaacatgttttaagaaagcgaaaggagcgggcgc

SEQ ID NO: 248 - DNA, 42 bp, synthetic construct
  misc_feature 1..42: note = source = /note="Description of Artificial Sequence: Synthetic oligonucleotide"
  source 1..42: mol_type = other DNA
taaagcactaaatcggaacccaacagttgattcccaattctg

SEQ ID NO: 249 - DNA, 42 bp, synthetic construct
  misc_feature 1..42: note = source = /note="Description of Artificial Sequence: Synthetic oligonucleotide"
  source 1..42: mol_type = other DNA
tctggaagtttcattccatattaaagggagcccccgatttag

SEQ ID NO: 250 - DNA, 42 bp, synthetic construct
  misc_feature 1..42: note = source = /note="Description of Artificial Sequence: Synthetic oligonucleotide"
  source 1..42: mol_type = other DNA
tagggcgctggcaagtgtagcagaggcttttgcaaaagaagt

SEQ ID NO: 251 - DNA, 42 bp, synthetic construct
  misc_feature 1..42: note = source = /note="Description of Artificial Sequence: Synthetic oligonucleotide"
  source 1..42: mol_type = other DNA
catagtaagagcaacactatcttttttggggtcgaggtgccg

SEQ ID NO: 252 - DNA, 42 bp, synthetic construct
  misc_feature 1..42: note = source = /note="Description of Artificial Sequence: Synthetic oligonucleotide"
  source 1..42: mol_type = other DNA
tgaaccatcacccaaatcaagataaccctcgtttaccagacg

SEQ ID NO: 253 - DNA, 42 bp, synthetic construct
  misc_feature 1..42: note = source = /note="Description of Artificial Sequence: Synthetic oligonucleotide"
  source 1..42: mol_type = other DNA
acgataaaaaccaaaatagcgggtcacgctgcgcgtaaccac

SEQ ID NO: 254 - DNA, 42 bp, synthetic construct
  misc_feature 1..42: note = source = /note="Description of Artificial Sequence: Synthetic oligonucleotide"
  source 1..42: mol_type = other DNA
tctaaaatatctttaggagcaataaatattcattgaatcccc

SEQ ID NO: 255 - DNA, 42 bp, synthetic construct
  misc_feature 1..42: note = source = /note="Description of Artificial Sequence: Synthetic oligonucleotide"
  source 1..42: mol_type = other DNA
gtccaatactgcggaatcgtcctaacaactaatagattagag

SEQ ID NO: 256 - DNA, 42 bp, synthetic construct
  misc_feature 1..42: note = source = /note="Description of Artificial Sequence: Synthetic oligonucleotide"
  source 1..42: mol_type = other DNA
gtattaacaccgcctgcaacaaaaatgtttagactggatagc

SEQ ID NO: 257 - DNA, 42 bp, synthetic construct
  misc_feature 1..42: note = source = /note="Description of Artificial Sequence: Synthetic oligonucleotide"
  source 1..42: mol_type = other DNA
cacacccgccgcgcttaatgcatcaagagtaatcttgacaag

SEQ ID NO: 258 - DNA, 42 bp, synthetic construct
  misc_feature 1..42: note = source = /note="Description of Artificial Sequence: Synthetic oligonucleotide"
  source 1..42: mol_type = other DNA
aaccggatattcattacccaatcagggcgatggcccactacg

SEQ ID NO: 259 - DNA, 42 bp, synthetic construct
  misc_feature 1..42: note = source = /note="Description of Artificial Sequence: Synthetic oligonucleotide"
  source 1..42: mol_type = other DNA
ccgtcaatagataatacatttaatcataagggaaccgaactg

SEQ ID NO: 260 - DNA, 42 bp, synthetic construct
  misc_feature 1..42: note = source = /note="Description of Artificial Sequence: Synthetic oligonucleotide"
  source 1..42: mol_type = other DNA
accaactttgaaagaggacagaaaacagaggtgaggcggtca

SEQ ID NO: 261 - DNA, 42 bp, synthetic construct
  misc_feature 1..42: note = source = /note="Description of Artificial Sequence: Synthetic oligonucleotide"
  source 1..42: mol_type = other DNA
atcaacaatagataagtcctgtgtccagacgacgacaataaa

SEQ ID NO: 262 - DNA, 42 bp, synthetic construct
  misc_feature 1..42: note = source = /note="Description of Artificial Sequence: Synthetic oligonucleotide"
  source 1..42: mol_type = other DNA
gcagaggcattttcgagccaggtatgttagcaaacgtagaaa

SEQ ID NO: 263 - DNA, 42 bp, synthetic construct
  misc_feature 1..42: note = source = /note="Description of Artificial Sequence: Synthetic oligonucleotide"
  source 1..42: mol_type = other DNA
aggaaacgcaataataacggattgctttgaataccaagttac

SEQ ID NO: 264 - DNA, 42 bp, synthetic construct
  misc_feature 1..42: note = source = /note="Description of Artificial Sequence: Synthetic oligonucleotide"
  source 1..42: mol_type = other DNA
gtcagatgaatatacagtaacaaaccaatcaataatcggctg

SEQ ID NO: 265 - DNA, 42 bp, synthetic construct
  misc_feature 1..42: note = source = /note="Description of Artificial Sequence: Synthetic oligonucleotide"
  source 1..42: mol_type = other DNA
tcctaatttacgagcatgtagagtaccttttacatcgggaga

SEQ ID NO: 266 - DNA, 42 bp, synthetic construct
  misc_feature 1..42: note = source = /note="Description of Artificial Sequence: Synthetic oligonucleotide"
  source 1..42: mol_type = other DNA
aacaataacggattcgcctgaatacccaaaagaactggcatg

SEQ ID NO: 267 - DNA, 42 bp, synthetic construct
  misc_feature 1..42: note = source = /note="Description of Artificial Sequence: Synthetic oligonucleotide"
  source 1..42: mol_type = other DNA
attaagactccttattacgcataataagagaatataaagtac

SEQ ID NO: 268 - DNA, 42 bp, synthetic construct
  misc_feature 1..42: note = source = /note="Description of Artificial Sequence: Synthetic oligonucleotide"
  source 1..42: mol_type = other DNA
cgacaaaaggtaaagtaattcaacaagaaaaataatatccca

SEQ ID NO: 269 - DNA, 42 bp, synthetic construct
  misc_feature 1..42: note = source = /note="Description of Artificial Sequence: Synthetic oligonucleotide"
  source 1..42: mol_type = other DNA
cattaaacgggtaaaatacgttgagtgaataaccttgcttct

SEQ ID NO: 270 - DNA, 42 bp, synthetic construct
  misc_feature 1..42: note = source = /note="Description of Artificial Sequence: Synthetic oligonucleotide"
  source 1..42: mol_type = other DNA
aaaatcgcgcagaggcgaattatggtttaccagcgccaaaga

SEQ ID NO: 271 - DNA, 42 bp, synthetic construct
  misc_feature 1..42: note = source = /note="Description of Artificial Sequence: Synthetic oligonucleotide"
  source 1..42: mol_type = other DNA
ataaaagaaacgcaaagacaccaacgccaacatgtaatttag

SEQ ID NO: 272 - DNA, 42 bp, synthetic construct
  misc_feature 1..42: note = source = /note="Description of Artificial Sequence: Synthetic oligonucleotide"
  source 1..42: mol_type = other DNA
gtgataaataaggcgttaaatagaatacactaaaacactcat

SEQ ID NO: 273 - DNA, 42 bp, synthetic construct
  misc_feature 1..42: note = source = /note="Description of Artificial Sequence: Synthetic oligonucleotide"
  source 1..42: mol_type = other DNA
acctaaaacgaaagaggcaaaaagaataaacaccggaatcat

SEQ ID NO: 274 - DNA, 42 bp, synthetic construct
  misc_feature 1..42: note = source = /note="Description of Artificial Sequence: Synthetic oligonucleotide"
  source 1..42: mol_type = other DNA
aattactagaaaaagcctgttggataagtgccgtcgagaggg

SEQ ID NO: 275 - DNA, 42 bp, synthetic construct
  misc_feature 1..42: note = source = /note="Description of Artificial Sequence: Synthetic oligonucleotide"
  source 1..42: mol_type = other DNA
gggttttgctcagtaccaggctagtatcatatgcgttataca

SEQ ID NO: 276 - DNA, 42 bp, synthetic construct
  misc_feature 1..42: note = source = /note="Description of Artificial Sequence: Synthetic oligonucleotide"
  source 1..42: mol_type = other DNA
tacatttaacaatttcatttgataggtgtatcaccgtactca

SEQ ID NO: 277 - DNA, 42 bp, synthetic construct
  misc_feature 1..42: note = source = /note="Description of Artificial Sequence: Synthetic oligonucleotide"
  source 1..42: mol_type = other DNA
ttgatataagtatagcccggaaattaccttttttaatggaaa

SEQ ID NO: 278 - DNA, 42 bp, synthetic construct
  misc_feature 1..42: note = source = /note="Description of Artificial Sequence: Synthetic oligonucleotide"
  source 1..42: mol_type = other DNA
aattcttaccagtataaagccgtattaagaggctgagactcc

SEQ ID NO: 279 - DNA, 42 bp, synthetic construct
  misc_feature 1..42: note = source = /note="Description of Artificial Sequence: Synthetic oligonucleotide"
  source 1..42: mol_type = other DNA
gtgcccgtataaacagttaatcatcaagaaaacaaaattaat

SEQ ID NO: 280 - DNA, 42 bp, synthetic construct
  misc_feature 1..42: note = source = /note="Description of Artificial Sequence: Synthetic oligonucleotide"
  source 1..42: mol_type = other DNA
aaaagaagatgatgaaacaaagccccctgcctatttcggaac

SEQ ID NO: 281 - DNA, 42 bp, synthetic construct
  misc_feature 1..42: note = source = /note="Description of Artificial Sequence: Synthetic oligonucleotide"
  source 1..42: mol_type = other DNA
ctattattctgaaacatgaaaaacgctcaacagtagggctta

SEQ ID NO: 282 - DNA, 42 bp, synthetic construct
  misc_feature 1..42: note = source = /note="Description of Artificial Sequence: Synthetic oligonucleotide"
  source 1..42: mol_type = other DNA
attgagaatcgccatatttaacacggaataagtttattttgt

SEQ ID NO: 283 - DNA, 42 bp, synthetic construct
  misc_feature 1..42: note = source = /note="Description of Artificial Sequence: Synthetic oligonucleotide"
  source 1..42: mol_type = other DNA
cacaatcaatagaaaattcatattcatttcaattacctgagc

SEQ ID NO: 284 - DNA, 42 bp, synthetic construct
  misc_feature 1..42: note = source = /note="Description of Artificial Sequence: Synthetic oligonucleotide"
  source 1..42: mol_type = other DNA
cagtacataaatcaatatatgaatgccactacgaaggcacca

SEQ ID NO: 285 - DNA, 42 bp, synthetic construct
  misc_feature 1..42: note = source = /note="Description of Artificial Sequence: Synthetic oligonucleotide"
  source 1..42: mol_type = other DNA
gtaaatcgtcgctattaattaacctgctccatgttacttagc

SEQ ID NO: 286 - DNA, 42 bp, synthetic construct
  misc_feature 1..42: note = source = /note="Description of Artificial Sequence: Synthetic oligonucleotide"
  source 1..42: mol_type = other DNA
agcgcgaaacaaagtacaacgatggtttgaaataccgaccgt

SEQ ID NO: 287 - DNA, 42 bp, synthetic construct
  misc_feature 1..42: note = source = /note="Description of Artificial Sequence: Synthetic oligonucleotide"
  source 1..42: mol_type = other DNA
tataactatatgtaaatgctgcaaatatcgcgttttaattcg

SEQ ID NO: 288 - DNA, 42 bp, synthetic construct
  misc_feature 1..42: note = source = /note="Description of Artificial Sequence: Synthetic oligonucleotide"
  source 1..42: mol_type = other DNA
aagaggaagcccgaaagacttatgcaaatccaatcgcaagac

SEQ ID NO: 289 - DNA, 42 bp, synthetic construct
  misc_feature 1..42: note = source = /note="Description of Artificial Sequence: Synthetic oligonucleotide"
  source 1..42: mol_type = other DNA
tagtgaatttatcaaaatcatggaagcaaactccaacaggtc

SEQ ID NO: 290 - DNA, 42 bp, synthetic construct
  misc_feature 1..42: note = source = /note="Description of Artificial Sequence: Synthetic oligonucleotide"
  source 1..42: mol_type = other DNA
agcttcaaagcgaaccagaccaggtctgagagactacctttt

SEQ ID NO: 291 - DNA, 42 bp, synthetic construct
  misc_feature 1..42: note = source = /note="Description of Artificial Sequence: Synthetic oligonucleotide"
  source 1..42: mol_type = other DNA
aaagaacgcgagaaaacttttctgactattatagtcagaagc

SEQ ID NO: 292 - DNA, 42 bp, synthetic construct
  misc_feature 1..42: note = source = /note="Description of Artificial Sequence: Synthetic oligonucleotide"
  source 1..42: mol_type = other DNA
ctcaaatgctttaaacagttctaagacgctgagaagagtcaa

SEQ ID NO: 293 - DNA, 42 bp, synthetic construct
  misc_feature 1..42: note = source = /note="Description of Artificial Sequence: Synthetic oligonucleotide"
  source 1..42: mol_type = other DNA
aaacatagcgatagcttagatagaaaacgagaatgaccataa

SEQ ID NO: 294 - DNA, 42 bp, synthetic construct
  misc_feature 1..42: note = source = /note="Description of Artificial Sequence: Synthetic oligonucleotide"
  source 1..42: mol_type = other DNA
atcaaaaatcaggtctttacctcaaatatattttagttaatt

SEQ ID NO: 295 - DNA, 42 bp, synthetic construct
  misc_feature 1..42: note = source = /note="Description of Artificial Sequence: Synthetic oligonucleotide"
  source 1..42: mol_type = other DNA
tcatcttctgacctaaatttagagatttgtatcatcgcctga

SEQ ID NO: 296 - DNA, 42 bp, synthetic construct
  misc_feature 1..42: note = source = /note="Description of Artificial Sequence: Synthetic oligonucleotide"
  source 1..42: mol_type = other DNA
taaattgtgtcgaaatccgcgattttcccttagaatccttga